(19) United States
(12) Patent Application Publication
(10) Pub. No.: US 2025/0312448 A1
(43) Pub. Date: Oct. 9, 2025

(54) ENHANCING THE ACTIVITY OF CELLULAR THERAPIES IN THE TUMOR MICROENVIRONMENT

(71) Applicant: BOARD OF REGENTS, THE UNIVERSITY OF TEXAS SYSTEM, Austin, TX (US)

(72) Inventors: Katy REZVANI, Houston, TX (US); Hind RAFEI, Houston, TX (US)

(73) Assignee: BOARD OF REGENTS, THE UNIVERSITY OF TEXAS SYSTEM, Austin, TX (US)

(21) Appl. No.: 18/865,045

(22) PCT Filed: Jun. 14, 2023
(86) PCT No.: PCT/US2023/068413

§ 371 Date: Nov. 12, 2024

Related U.S. Application Data
(60) Provisional application No. 63/352,516, filed on Jun. 15, 2022.

(51) Int. Cl. A61K 40/15; A61K 40/31; A61K 40/42; A61P 35/00; C07K 14/47; C07K 16/28; C07K 16/30; C12N 5/0783; C12N 9/22; C12N 15/11; C12N 15/90

(52) U.S. Cl. CPC A61K 40/15; A61K 40/31; A61K 40/42; A61P 35/00; C07K 14/4702; C07K 16/2875; C07K 16/30; C12N 5/0646; C12N 9/226; C12N 15/11; C12N 15/907; C12N 2310/20; C12N 2510/00

(57) ABSTRACT

Embodiments of the disclosure encompass improvements on cell therapies by allowing the cells to be more effective for cancer treatment, including in a solid tumor microenvironment. In specific cases, the cells are modified to have reduced or inhibited levels of expression of GPR4, GPR31, GPR68, GPR81, GPR132, GPR151, CREM, ICER, or CREB1, such as by CRISPR gene editing. In certain cases, the cells are modified to have reduced or inhibited levels of expression of CREM. In certain cases, the cells are further modified to express, for example, one or more engineered receptors, one or more cytokines, and/or optionally one or more suicide genes.

### CROSS REFERENCE TO RELATED APPLICATIONS

This application claims priority to U.S. Provisional Patent Application Ser. No. 63/352,516 filed Jun. 15, 2022, which is incorporated by reference herein in its entirety.

### SEQUENCE LISTING

The instant application contains a Sequence Listing which has been submitted in ST26 format and is hereby incorporated by reference in its entirety. Said ST26 copy, created on Jun. 11, 2023, is named MDAC_P1332WO_Sequence_Listing.xml and is 240,688 bytes in size.

### TECHNICAL FIELD

Embodiments of the disclosure include at least the fields of cell biology, molecular biology, immunology, and medicine, including cancer medicine.

### BACKGROUND

In the wake of the 2017 Food and Drug Administration (FDA) approvals of chimeric antigen receptor (CAR)-T-cell therapies for the treatment of patients with lymphoma and leukemia, adoptive cellular therapies have rapidly become a focal point for stakeholders across the field of cancer immunotherapy. While this treatment modality had displayed unprecedented patient responses and offers a significant curative potential for certain hematological malignancies, success in solid tumors remains elusive, in part because of the unique features of the solid tumor microenvironment (TME) characterized by hypoxia, acidic pH, nutrition depletion, and immunosuppression (see e.g., Renner et al., 2017). Acidity is a prominent feature of the tumor microenvironment primarily due to of acidic metabolites, e.g., lactic acid caused by active glycolysis under hypoxic conditions (see e.g., Huber et al., 2017). Acidity mediates immunosuppression, tumor progression, and poor prognosis. Specifically, tissue acidosis leads to suppression of immune cell-mediated responses, such as a decrease in natural killer (NK)- and T-cell cytoxicity, cytokine production, and tumor surveillance.

Provided herein are technologies for overcoming the immunosuppressive phenotypes associated with acidic environments and/or the TME.

The present disclosure provides solutions to long-felt needs in the art of cancer therapy by manipulating the CAMP signaling pathway through engineered mutation of G-protein coupled receptor 4 (GPR4), G-protein coupled receptor 31 (GPR31), G-protein coupled receptor 68 (GPR68), G-protein coupled receptor 81 (GPR81), G-protein coupled receptor 132 (GPR132), G-protein coupled receptor 151 (GPR151), cAMP response element modulator (CREM), inducible cAMP early repressor (ICER), and/or cyclic AMP-responsive element-binding protein 1 (CREB1) genes in order to facilitate immune effector cell activity in the solid tumor microenvironment.

### BRIEF SUMMARY

Embodiments of the disclosure include methods and compositions associated with cell therapy, including adoptive cell therapy. Particular embodiments of the disclosure encompass methods and compositions for cancer immunotherapy, anti-pathogen immunotherapy, or both. Pathogens include at least viruses, bacteria, fungi, and parasites. The disclosure encompasses immune effector cell therapies that have been improved for the explicit purpose of imparting one or more characteristics to the cells that improves their efficacy. In specific embodiments, immune effector cells are modified to allow them to better kill target cells, such as cancer cells. In specific embodiments, immune effector cells are engineered to have reduced expression of one or more gene products that allow the engineered cells to be effective in an acidic environment, such as a solid tumor microenvironment, as compared to in the absence of the engineering, although the cells are also effective for cancers that lack solid tumors, such as hematological cancers. In particular embodiments, the engineered cells are better equipped to be effective to kill cancer cells in environments that are hypoxic, that have an acidic pH, that have nutrition depletion, and/or that experience immunosuppression.

In particular embodiments, immune effector cells are comprised in compositions and are used in methods encompassed herein that have been engineered to have reduced level of expression of G-protein coupled receptor 4 (GPR4), G-protein coupled receptor 31 (GPR31), G-protein coupled receptor 68 (GPR68), G-protein coupled receptor 81 (GPR81), G-protein coupled receptor 132 (GPR132), G-protein coupled receptor 151 (GPR151), CAMP response element modulator (CREM), inducible cAMP early repressor (ICER), and/or cyclic AMP-responsive element-binding protein 1 (CREB1). In some embodiments, immune effector cells have full inhibition of expression of GPR4, GPR31, GPR68, GPR81, GPR132, GPR151, CREM, ICER, and/or CREB1, such as lacking detectable expression of the aforementioned genes through routine methods in the art.

In particular embodiments, the endogenous GPR4, GPR31, GPR68, GPR81, GPR132, GPR151, CREM, ICER, and/or CREB1 gene has been modified by genetic manipulation of the genomic locus of GPR4, GPR31, GPR68, GPR81, GPR132, GPR151, CREM, ICER, and/or CREB1. The immune effector cells having reduced or fully inhibited expression of GPR4, GPR31, GPR68, GPR81, GPR132, GPR151, CREM, ICER, and/or CREB1 may or may not be modified in an additional manner by the hand of man, such as expressing one or more exogenously provided gene products. In specific embodiments, the gene product is a receptor, cytokine, chemokine, suicide gene, or combination thereof. In particular cases, the receptor is an antigen receptor, wherein the antigen may or may not be a cancer antigen, including an antigen on solid tumor cells. In specific cases, the antigen receptor is a chimeric antigen receptor (CAR) or a non-natural T-cell receptor.

In some embodiments, the present disclosure knocks out or knocks down the gene encoding GPR4, GPR31, GPR68, GPR81, GPR132, GPR151, CREM, ICER, and/or CREB1 from immune effector cells used in various cellular therapies to render them insensitive to the immunosuppressive effects of acidity and, hence, increase their survival, proliferation, and immune function, including at least in the acidic solid tumor microenvironment. Using the gene-editing CRISPR/Cas9 technology, as one example, the feasibility is confirmed of knocking-out the CAMP sensing and/or signaling pathway, utilizing Cas9 preloaded with chemically synthesized crFNA: tracrRNA duplex targeting CREM. Data disclosed herein demonstrate that knocking-out CREM from NK cells leads to improvement in their cytotoxic effects as well as antitumor activity against cell lines of cancers characterized by active glycolysis and prominent acidosis of their microenvironment. In some embodiments, the genetic engineering strategy targeting CREM could be combined with different forms of cellular therapies, including CAR-T cells, CAR-NK cells, T-cell receptor (TCR)-T cells, T-cell receptor (TCR)-NK cells, tumor-infiltrating lymphocytes (TILs), or a combination thereof, to potentiate them against various types of cancers, including solid tumors.

The immune effector cells that are engineered may be of any kind, but in specific embodiments the immune effector cells are T cells, natural killer (NK) cells, NK T cells, macrophages, B cells, tumor-infiltrating lymphocytes, dendritic cells, mesenchymal stem cells (MSCs), a combination thereof, and so forth. In particular cases, the immune effector cells are NK cells, including cord blood-derived NK cells.

Any medical conditions may be treated by administration of a therapeutically effective amount of the engineered immune effector cells of the encompassed disclosure. In particular embodiments, the cells are utilized in compositions for treatment of cancer of any kind.

The present disclosure concerns novel strategies utilizing gene-editing technology (e.g., CRISPR/Cas9) to knock-out GPR4, GPR31, GPR68, GPR81, GPR132, GPR151, CREM, ICER, and/or CREB1 gene from immune cells to empower them and enhance their antitumor activity as cellular therapies against cancers of any kind, including at least solid tumors.

Embodiments of the disclosure include compositions and uses thereof regarding engineered immune effector cells, wherein the endogenous GPR4, GPR31, GPR68, GPR81, GPR132, GPR151, CREM, ICER, and/or CREB1 gene in the cell is reduced or inhibited fully in expression. In specific embodiments, the cell is a T cell, NK cell, NK T cell, macrophage, B cell, invariant NKT cell, gamma delta T cell, MSC, tumor-infiltrating lymphocyte, dendritic cell, or a mixture thereof. In a specific embodiment, the NK cell is derived from cord blood. In some cases, the cell comprises one or more engineered receptors, including an engineered antigen receptor such as a CAR, chemokine receptor, homing receptor, and/or a non-natural T cell receptor. The antigen may be a cancer antigen, including a solid tumor antigen. Specific examples of antigens including an antigen selected from the group consisting of 5T4, 8H9, αvβ6 integrin, BCMA, B7-H3, B7-H6, CAIX, CA9, CD5, CD19, CD20, CD22, CD30, CD33, CD38, CD44, CD44v6, CD44v7/8, CD70, CD123, CD138, CD171, CEA, CSPG4, CS1, CLL1, CD99, DLL3, EGFR, EGFR family including ErbB2 (HER2), EGFRVIII, EGP2, EGP40, ERBB3, ERBB4, ErbB3/4, EPCAM, EphA2, EpCAM, FAP, FBP, fetal AchR, FRα, GD2, GD3, Glypican-3 (GPC3), HLA-A1+MAGE1, HLA-A1+NY-ESO-1, IL-11Rα, IL-13Rα2, Lambda, Lewis-Y, LICAM, Kappa, KDR, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, TROP2, HMW-MAA, VEGFR2, and a combination thereof (the receptor may have two or more antigen binding domains that bind different antigens).

In certain embodiments, the cell comprises expression of one or more exogenous chemokines or one or more cytokines. Examples of cytokines includes IL-15, IL-12, IL-21, IL-2, IL-18, IL-7, or a combination thereof. In addition, or alternatively, the cell comprises a suicide gene.

The endogenous GPR4, GPR31, GPR68, GPR81, GPR132, GPR151, CREM, ICER, and/or CREB1 gene may be reduced or inhibited in expression from homologous recombination or non-homologous recombination. In certain cases, the endogenous GPR4, GPR31, GPR68, GPR81, GPR132, GPR151, CREM, ICER, and/or CREB1 gene is knocked out by CRISPR-Cas9. Any cells of the disclosure include cells that are autologous, allogeneic, or xenogeneic with respect to a recipient individual.

In specific embodiments, the cell is further reduced or inhibited in expression of one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD38, CD40, IL21R, ICAM1, CD95, CD80, CD86, IL10R, CD5, GR, and CD7.

Embodiments of the disclosure include populations of any one of the cells encompassed herein. In specific embodiments, the population is comprised in a pharmaceutically acceptable excipient.

Specific embodiments of the disclosure include methods of engineering NK cells so that their functionality is improved in any manner, including in a non-transient manner, and with respect to NK cells that are not so engineered. In specific embodiments, the gene modification in NK cells results in the cells having enhanced cytotoxicity towards cancer cells and/or having enhanced expansion, persistence and/or proliferation compared to NK cells that are not so engineered. Methods of the disclosure include methods of suppression of immune cell-mediated responses in vivo in an individual receiving adoptive cell therapy of any kind, including with T cells and/or NK cells, merely as examples, and in which case the cells are engineered to have reduced or fully inhibited expression of GPR4, GPR31, GPR68, GPR81, GPR132, GPR151, CREM, ICER, and/or CREB1.

Embodiments of the disclosure include improvement of adoptive cell therapy of any kind in a tumor microenvironment by utilizing engineered cells as encompassed herein, compared to cells that have not been so engineered. In specific embodiments, the disclosure includes production and use of immune effector cells that have enhanced cytotoxicity, persistence, and expansion because of engineered (as opposed to natural to the cells) reduced or fully inhibited expression of endogenous GPR4, GPR31, GPR68, GPR81, GPR132, GPR151, CREM, ICER, and/or CREB1 in the cells, compared to cells that do not have engineering to result in reduced or fully inhibited expression of endogenous GPR4, GPR31, GPR68, GPR81, GPR132, GPR151, CREM, ICER, and/or CREB1 in the cells.

Immune effector cells of any kind, such as NK cells, can be obtained from a number of non-limiting sources, including from peripheral blood mononuclear cells, bone marrow, lymph node tissue, cord blood, thymus tissue, tissue from a site of infection, ascites, pleural effusion, spleen tissue, tumors, or commercially available. Any number of immune cell lines available and known to those skilled in the art, may be used.

In addition to the immune effector cells being engineered to have reduced or fully inhibited expression of endogenous GPR4, GPR31, GPR68, GPR81, GPR132, GPR151, CREM, ICER, and/or CREB1, in at least some cases the engineered immune effector cells are engineered in one or more other aspects. In specific embodiments, the cells are also engineered to express one or more engineered receptors (as opposed to receptors that are endogenous to the cells), one or more cytokines, and/or one or more suicide genes. The engineered receptors may be of any kind, including at least one or more CARs, one or more T cell receptors, one or more chemokine receptors, a combination thereof, and so forth. Any engineering of the immune effector cells may or may not occur after the knock out (or knock down) of expression of GPR4, GPR31, GPR68, GPR81, GPR132, GPR151, CREM, ICER, and/or CREB1. In cases wherein the engineered immune effector cells having reduced or fully inhibited expression of GPR4, GPR31, GPR68, GPR81, GPR132, GPR151, CREM, ICER, and/or CREB1 also are engineered to express two or more other genes, the engineering for expression of the two or more other genes may or may not occur at the same time as each other. For example, in cases wherein the GPR4, GPR31, GPR68, GPR81, GPR132, GPR151, CREM, ICER, and/or CREB1 knock-out (KO) (or knock down) cells are engineered to express a CAR and a cytokine, the engineering to express the CAR and the cytokine may or may not occur at substantially the same time. Any other transgenes for the cells may or may not be expressed from the same vector. In illustrative cases, a CAR, and a cytokine (as representatives only) may or may not be expressed from the same vector upon transfection or transformation of the immune effector cells.

Embodiments of the disclosure include methods of treating cancer in an individual, comprising the step of administering a therapeutically effective amount of the population of cells of the disclosure to the individual. In some cases, the cancer is a solid tumor or is not a solid tumor. The cancer may be of the lung, brain, breast, blood, skin, pancreas, liver, colon, head and neck, kidney, thyroid, stomach, spleen, gallbladder, bone, ovary, testes, endometrium, prostate, rectum, anus, or cervix. The individual may be a mammal, such as a human, dog, cat, horse, cow, sheep, pig, or rodent. The individual may or may not be administered an additional cancer therapy, such as surgery, radiation, chemotherapy, hormone therapy, immunotherapy, or a combination thereof. In specific embodiments, the method further comprises the step of diagnosing cancer in the individual. In some cases, the method further comprises the step of generating the population of cells. The cells may be autologous or allogeneic with respect to the individual.

In specific embodiments, the cells are NK cells, such as cord blood NK cells, including those that express one or more engineered antigen receptors. The cells may be CAR-expressing NK cells or TCR-expressing NK cells.

In certain embodiments, disclosed herein are engineered immune effector cells, wherein the cell comprises one or more engineered mutations in an endogenous G-protein coupled receptor 4 (GPR4), G-protein coupled receptor 31 (GPR31), G-protein coupled receptor 68 (GPR68), G-protein coupled receptor 81 (GPR81), G-protein coupled receptor 132 (GPR132), G-protein coupled receptor 151 (GPR151), cAMP response element modulator (CREM), inducible cAMP early repressor (ICER), and/or cyclic AMP-responsive element-binding protein 1 (CREB1) gene in the cell. In certain embodiments, a mutation is a partial or complete loss of function, and/or knock-out (KO) mutation. In certain embodiments, a mutation reduces or inhibits transcription or post-transcriptional processing of one or more mRNA isoforms encoded by the mutated endogenous gene relative to a non-mutated locus encoding the same endogenous gene. In certain embodiments, a mutation is a neomorphic or gain of function mutation. In certain embodiments, a mutation increases transcription or post-transcriptional processing of one or more mRNA isoforms encoded by the mutated endogenous gene relative to a non-mutated locus encoding the same endogenous gene. In certain embodiments, a mutation results in a modified mRNA isoform population encoded by the mutated endogenous gene relative to a representative mRNA population encoded by a non-mutated locus encoding the same endogenous gene. In certain embodiments, a mutation results in a modified protein isoform population encoded by the mutated endogenous gene relative to a representative protein population encoded by a non-mutated locus encoding the same endogenous gene.

In certain embodiments, a mutation results in improved cytotoxicity of the engineered cell in an acidic environment and/or a tumor microenvironment (TME) relative to a control non-engineered cell. In certain embodiments, a mutation results in improved cytotoxicity of the engineered cell in an acidic environment with a pH less than or equal to about 7.0 relative to control non-engineered cell. In certain embodiments, a mutation results in improved cytotoxicity of the engineered cell in an acidic environment with a pH less than or equal to about 5.9 relative to a control non-engineered cell. In certain embodiments, a mutation results in enhanced polyfunctionality of the engineered cell relative to a control non-engineered cell in response to stimulation by tumor cells. In certain embodiments, enhanced polyfunctionality is evidenced by an increase in cytokine release in response to stimulation by tumor cells. In certain embodiments, polyfunctionality is evidenced by an increase in cytokine release comprises an increase in interferon gamma (IFN-g), tumor necrosis factor alpha (TNF-a), and/or the degranulation marker CD107a, in response to stimulation by tumor cells. In certain embodiments, polyfunctionality is evidenced by an increase in cytokine release comprises an increase in granulocyte-macrophage colony-stimulating factor (GMCSF), soluble CD137 (sCD137), INF-g, Granzyme A, interleukin 13 (IL-13), Granzyme B, soluble FAS cell surface death receptor (sFas), interleukin 6 (IL-6), soluble FAS cell surface death receptor ligand (sFasL), macrophage inflammatory protein-1 alpha (MIP-1a), macrophage inflammatory protein-1 beta (MIP-1b), TNF-a, and/or Perforin, in response to stimulation by tumor cells.

In certain embodiments, a mutation provides an enhanced activated and/or cytotoxic phenotype for the engineered cell relative to a control non-engineered cell. In some embodiments, a mutation leads to an enhanced activated and/or cytotoxic phenotype for the engineered cell relative to a control non-engineered cell, wherein the enhanced activated and/or cytotoxic phenotype is associated with one or more of GSEA identified pathways: G2M checkpoint, E2F targets, P53 pathway, Mitotic spindle, MYC, MTORC1, Androgen Response, Unfolded Protein Response, Spermatogenesis, Heme Metabolism, TNFalpha signaling, Protein Secretion, Apoptosis, Oxidative Phosphorylation, DNA Repair, UV Response, and/or Estrogen Response Early. In some embodiments, a mutation leads to upregulation of G2M, E2F, MYC, MTORC1, oxidative phosphorylation, and/or TNFa signaling. In certain embodiments, a mutation provides an enhanced proliferative capacity and/or persistence phenotype for the engineered cell relative to a control non-engineered cell. In certain embodiments, an enhanced proliferative capacity and/or persistence occurs in the absence of stimulation by exogenous interleukin 2 (IL-2). In certain embodiments, an enhanced proliferative capacity and/or persistence does not result in autonomous growth. In certain embodiments, a mutation provides an enhanced metabolic fitness phenotype for the engineered cell relative to a control non-engineered cell. In certain embodiments, an enhanced metabolic fitness is a higher glycolytic capacity and/or improved oxygen consumption rate (OCR).

In certain embodiments, a mutation in an endogenous gene is in the gene CREM. In certain embodiments, a CREM mutation results in a decrease in expression of CREM RNA isoforms CREM-228 (ICER), CREM-207, CREM-230, CREM-211, CREM-213, CREM-239, CREM-201, CREM-232, CREM-217, and/or CREM-225. In certain embodiments, a CREM mutation results in an increase in expression of CREM RNA isoform CREM-218. In certain embodiments, a CREM mutation is a result of exposure of the cell to a polynucleotide comprising the sequence of SEQ ID NO: 140 and/or SEQ ID NO: 142. In certain embodiments, a CREM mutation results in a decrease in one or more CREM protein isoforms by greater than 30%, 35%, 40%, 45%, 50%, 55%, 60%, 65%, 70%, 75%, 80%, 85%, 90%, 95%, 96%, 97%, 98%, 99%, or 100%, or any range derivable therein. In certain embodiments, a CREM mutation results in a decrease in one or more CREM protein isoforms by greater than 50%, 51%, 52%, 53%, 54%, 55%, 56%, 57%, 58%, 59%, 60%, 61%, 62%, 63%, 64%, 65%, 66%, 67%, 68%, 69%, and/or 70%. In certain embodiments, a CREM mutation results in a decrease in one or more CREM protein isoforms by greater than 71%, 72%, 73%, 74%, 75%, 76%, 77%, 78%, 79%, 80%, 81%, 82%, 83%, 84%, 85%, 86%, 87%, 88%, 89%, or 90%.

In certain embodiments, the cell has further been conditioned to acidic environments by contacting the cell ex vivo with acidic stimuli. In some embodiments, the acidic stimuli is provided at a concentration of greater than or equal to about 1, 2, 3, 4, 5, 6, 7, 8, 9, or 10 mM. In some embodiments, the acidic stimuli is provided at a concentration of greater than or equal to about 2 to 3 mM. In some embodiments, the acidic stimuli is provided at a concentration of greater than or equal to about 2.5 mM. In some embodiments, the conditioning to acidic environments is by incremental and/or cumulative contacting with acidic stimuli. In some embodiments, the cells are conditioned over a period of at least about 10 to 18 days, optionally at least about 14 days. In some embodiments, cell conditioning comprises addition of acidic stimuli about every 48-72 hours, optionally about every 48 hours. In some embodiments, the acidic stimuli comprises or consists essentially of lactic acid. In some embodiments, the cell is conditioned to acidic environments of less than or equal to about pH 6.0.

In certain embodiments, a cell is a T cell, natural killer (NK) cell, NK T cell, macrophage, B cell, invariant NKT cells, gamma delta T cells, MSCs, tumor-infiltrating lymphocyte, or dendritic cell. In certain embodiments, a cell is a NK cell derived from cord blood (CB), peripheral blood (PB), an NK cell line, bone marrow, stem cells, or a mixture thereof. In certain embodiments, an NK cell is derived from cord blood.

In certain embodiments, a cell comprises one or more engineered receptors. In certain embodiments, an engineered receptor comprises an engineered antigen receptor. In certain embodiments, an engineered antigen receptor is a chimeric antigen receptor (CAR) and/or a T cell receptor (TCR). In certain embodiments, an engineered antigen receptor is a CAR. In certain embodiments, an antigen is a cancer antigen. In certain embodiments, an antigen is a solid tumor antigen. In certain embodiments, an antigen is selected from the group consisting of 5T4, 8H9, αvβ6 integrin, BCMA, B7-H3, B7-H6, CAIX, CA9, CD5, CD19, CD20, CD22, CD30, CD33, CD38, CD44, CD44v6, CD44v7/8, CD70, CD123, CD138, CD171, CEA, CSPG4, CS1, CLL1, CD99, DLL3, EGFR, EGFR family including ErbB2 (HER2), EGFRVIII, EGP2, EGP40, ERBB3, ERBB4, ErbB3/4, EPCAM, EphA2, EpCAM, FAP, FBP, fetal AchR, FRα, GD2, GD3, Glypican-3 (GPC3), HLA-A1+MAGE1, HLA-A1+NY-ESO-1, IL-11Rα, IL-13Rα2, Lambda, Lewis-Y, LICAM, Kappa, KDR, MCSP, Mesothelin, Muc1, Muc16, NCAM, NKG2D Ligands, NY-ESO-1, PRAME, PSC1, PSCA, PSMA, ROR1, SP17, Survivin, TAG72, TROP2, TEMs, HMW-MAA, VEGFR2, and a combination thereof.

In certain embodiments, one or more engineered receptors comprises a cytokine receptor, chemokine receptor, homing receptor, or a combination thereof. In certain embodiments, a cell comprises expression of one or more exogenous chemokines and/or one or more cytokines. In certain embodiments, a cytokine is IL-15, IL-12, IL-21, IL-2, IL-18, IL-7, or a combination thereof. In certain embodiments, a cytokine is IL-15. In certain embodiments, a cell comprises a suicide gene.

In certain embodiments, an endogenous gene is mutated as a result of homologous recombination or non-homologous recombination. In certain embodiments, an endogenous gene is mutated by an endonuclease. In certain embodiments, an endonuclease is an RNA guided endonuclease. In certain embodiments, an RNA guided endonuclease is CRISPR-Cas9. In certain embodiments, a cell comprises one or more additional mutations in one or more genes, wherein the gene is selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD38, CD40, IL21R, ICAM1, CD95, CD80, CD86, IL10R, CD5, GR, and CD7.

Also disclosed herein are populations of any of the cells described herein. In some embodiments, a population of cells is comprised in a pharmaceutically acceptable excipient.

Also disclosed herein are methods of treating cancer in an individual comprising the step of administering a therapeutically effective amount of a population of cells of claim to the individual. In some embodiments, cells are autologous, allogeneic, or xenogeneic with respect to the individual. In some embodiments, cells are allogeneic with respect to the individual. In some embodiments, a cancer comprises a solid tumor. In some embodiments, a cancer does not comprise a solid tumor. In some embodiments, a cancer is of the lung, brain, breast, blood, skin, pancreas, liver, colon, head and neck, kidney, thyroid, stomach, spleen, gallbladder, bone, ovary, testes, endometrium, prostate, rectum, anus, and/or cervix. In some embodiments, an individual is a mammal. In some embodiments, an individual is a human, dog, cat, horse, cow, sheep, pig, or rodent. In some embodiments, an individual is a human. In some embodiments, an individual is administered an additional cancer therapy. In some embodiments, an additional cancer therapy is surgery, radiation, chemotherapy, hormone therapy, immunotherapy, or a combination thereof. In some embodiments, an individual is diagnosed with cancer.

Also disclosed herein are methods of engineering an immune effector cell. In some embodiments, methods of engineering an immune effector cell comprises mutating an endogenous CAMP response element modulator (CREM), G-protein coupled receptor 4 (GPR4), G-protein coupled receptor 31 (GPR31), G-protein coupled receptor 68 (GPR68), G-protein coupled receptor 81 (GPR81), G-protein coupled receptor 132 (GPR132), G-protein coupled receptor 151 (GPR151), inducible cAMP early repressor (ICER), and/or cyclic AMP-responsive element-binding protein 1 (CREB1) gene in the cell. In some embodiments, the mutating generates a partial or complete loss of function, and/or knock-out (KO) mutation. In some embodiments, the mutating reduces or inhibits transcription or post-transcriptional processing of one or more mRNA isoforms encoded by the mutated endogenous gene relative to a non-mutated locus encoding the same endogenous gene. In some embodiments, the mutating generates a neomorphic or gain of function mutation. In some embodiments, the mutating increases transcription or post-transcriptional processing of one or more mRNA isoforms encoded by the mutated endogenous gene relative to a non-mutated locus encoding the same endogenous gene. In some embodiments, the mutating generates a modified mRNA isoform population encoded by the mutated endogenous gene relative to a representative mRNA population encoded by a non-mutated locus encoding the same endogenous gene. In some embodiments, the mutating generates a modified protein isoform population encoded by the mutated endogenous gene relative to a representative protein population encoded by a non-mutated locus encoding the same endogenous gene.

In some embodiments, the mutating generates an improved cytotoxicity of the engineered cell in an acidic environment and/or a tumor microenvironment (TME) relative to a control non-engineered cell. In some embodiments, the mutating generates an improved cytotoxicity of the engineered cell in an acidic environment with a pH less than or equal to about 7.0 relative to control non-engineered cell. In some embodiments, the mutating generates an improved cytotoxicity of the engineered cell in an acidic environment with a pH less than or equal to about 5.9 relative to a control non-engineered cell.

In some embodiments, the mutating generates an enhanced polyfunctionality of the engineered cell relative to a control non-engineered cell in response to stimulation by tumor cells. In some embodiments, the enhanced polyfunctionality is evidenced by an increase in cytokine release in response to stimulation by tumor cells. In some embodiments, the increase in cytokine release comprises an increase in interferon gamma (IFN-g), tumor necrosis factor alpha (TNF-a), and/or the degranulation marker CD107a, in response to stimulation by tumor cells. In some embodiments, the increase in cytokine release comprises an increase in granulocyte-macrophage colony-stimulating factor (GMCSF), soluble CD137 (sCD137), INF-g, Granzyme A, interleukin 13 (IL-13), Granzyme B, soluble FAS cell surface death receptor (sFas), interleukin 6 (IL-6), soluble FAS cell surface death receptor ligand (sFasL), macrophage inflammatory protein-1 alpha (MIP-1a), macrophage inflammatory protein-1 beta (MIP-1b), TNF-α, and/or Perforin, in response to stimulation by tumor cells.

In some embodiments, the mutating generates an enhanced activated and/or cytotoxic phenotype for the engineered cell relative to a control non-engineered cell. In some embodiments, the mutating provides an enhanced activated and/or cytotoxic phenotype for the engineered cell relative to a control non-engineered cell, wherein the enhanced activated and/or cytotoxic phenotype is associated with one or more of GSEA identified pathways: G2M checkpoint, E2F targets, P53 pathway, Mitotic spindle, MYC, MTORC1, Androgen Response, Unfolded Protein Response, Spermatogenesis, Heme Metabolism, TNFalpha signaling, Protein Secretion, Apoptosis, Oxidative Phosphorylation, DNA Repair, UV Response, and/or Estrogen Response Early. In some embodiments, the mutating leads to upregulation of G2M, E2F, MYC, MTORC1, oxidative phosphorylation, and/or TNFa signaling.

In some embodiments, the mutating generates an enhanced proliferative capacity and/or persistence phenotype for the engineered cell relative to a control non-engineered cell. In some embodiments, the enhanced proliferative capacity and/or persistence occurs in the absence of stimulation by exogenous interleukin 2 (IL-2). In some embodiments, the enhanced proliferative capacity and/or persistence does not result in autonomous growth. In some embodiments, the mutating generates an enhanced metabolic fitness phenotype for the engineered cell relative to a control non-engineered cell. In some embodiments, the enhanced metabolic fitness is a higher glycolytic capacity and/or improved oxygen consumption rate (OCR).

In some embodiments, methods of engineering an immune effector cell comprises mutating an endogenous cAMP response element modulator (CREM) gene. In some embodiments, the mutating of CREM results in a decrease in expression of CREM RNA isoforms CREM-228 (ICER), CREM-207, CREM-230, CREM-211, CREM-213, CREM-239, CREM-201, CREM-232, CREM-217, and/or CREM-225. In some embodiments, the mutating of CREM results in an increase in expression of CREM RNA isoform CREM-218. In some embodiments, the mutating of CREM comprises exposure of the cell to a polynucleotide comprising the sequence of SEQ ID NO: 140 and/or SEQ ID NO: 142. In some embodiments, the mutating of CREM generates a decrease in one or more CREM protein isoforms by greater than 30%, 35%, 40%, 45%, 50%, 55%, 60%, 65%, 70%, 75%, 80%, 85%, 90%, 95%, 96%, 97%, 98%, 99%, or 100%, or any range derivable therein. In some embodiments, the mutating of CREM generates a decrease in one or more CREM protein isoforms by greater than 80%.

In certain embodiments, methods further comprise conditioning the cell to acidic environments by contacting the cell ex vivo with acidic stimuli. In some embodiments, conditioning the cell to acidic environments comprises providing the acidic stimuli at a concentration of greater than or equal to about 2 to 3 mM, optionally greater than or equal to about 2.5 mM. In some embodiments, the conditioning to acidic environments is by incremental and/or cumulative contacting with acidic stimuli. In some embodiments, the conditioning is over a period of at least about 10 to 18 days, optionally at least about 14 days. In some embodiments, the conditioning comprises addition of acidic stimuli about every 48-72 hours, optionally about every 48 hours. In some embodiments, the acidic stimuli comprises or consists essentially of lactic acid. In some embodiments, the conditioning is to acidic environments of less than or equal to about pH 6.0.

In some embodiments, methods of engineering an immune effector cell comprises mutating an endogenous gene in a T cell, natural killer (NK) cell, NK T cell, macrophage, B cell, invariant NKT cells, gamma delta T cells, MSCs, tumor-infiltrating lymphocyte, dendritic cell, or precursor cell thereof. In some embodiments, the cell is a NK cell derived from cord blood (CB), peripheral blood (PB), an NK cell line, bone marrow, stem cells, or a mixture thereof. In some embodiments, the NK cell is derived from cord blood.

In some embodiments, methods of engineering an immune effector cell comprises mutating an endogenous gene in a cell, wherein the cell also comprises one or more engineered receptors. In some embodiments, the one or more engineered receptors comprises an engineered antigen receptor. In some embodiments, the engineered antigen receptor is a chimeric antigen receptor (CAR) and/or a T cell receptor (TCR). In some embodiments, the engineered antigen receptor is a CAR. In some embodiments, the antigen is a cancer antigen. In some embodiments, the antigen is a solid tumor antigen. In some embodiments, the antigen is selected from the group consisting of 5T4, 8H9, αvβ6 integrin, BCMA, B7-H3, B7-H6, CAIX, CA9, CD5, CD19, CD20, CD22, CD30, CD33, CD38, CD44, CD44v6, CD44v7/8, CD70, CD123, CD138, CD171, CEA, CSPG4, CS1, CLL1, CD99, DLL3, EGFR, EGFR family including ErbB2 (HER2), EGFRVIII, EGP2, EGP40, ERBB3, ERBB4, ErbB3/4, EPCAM, EphA2, EpCAM, FAP, FBP, fetal AchR, FRα, GD2, GD3, Glypican-3 (GPC3), HLA-A1+MAGE1, HLA-A1+NY-ESO-1, IL-11Rα, IL-13Rα2, Lambda, Lewis-Y, L1CAM, Kappa, KDR, MCSP, Mesothelin, Muc1, Muc16, NCAM, NKG2D Ligands, NY-ESO-1, PRAME, PSC1, PSCA, PSMA, ROR1, SP17, Survivin, TAG72, TROP2, TEMs, HMW-MAA, VEGFR2, and a combination thereof. In some embodiments, the one or more engineered receptors comprises a cytokine receptor, chemokine receptor, homing receptor, or a combination thereof.

In some embodiments, methods of engineering an immune effector cell comprises mutating an endogenous gene in a cell, wherein the cell also comprises expression of one or more exogenous chemokines and/or one or more cytokines. In some embodiments, the cytokine is IL-15, IL-12, IL-21, IL-2, IL-18, IL-7, or a combination thereof. In some embodiments, the cytokine is IL-15. In some embodiments, wherein the cell comprises a suicide gene. In some embodiments, the mutating of the endogenous gene is comprises homologous recombination or non-homologous recombination. In some embodiments, the mutating of the endogenous gene is mediated by an endonuclease. In some embodiments, the endonuclease is an RNA guided endonuclease. In some embodiments, the RNA guided endonuclease is CRISPR-Cas9. In some embodiments, the cell comprises one or more additional mutations in one or more genes, wherein the gene is selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD38, CD40, IL21R, ICAM1, CD95, CD80, CD86, IL10R, CD5, GR, and CD7.

The following aspects describe certain inventions disclosed herein.
Aspect 1 is an engineered immune effector cell, wherein the cell comprises one or more engineered mutations in an endogenous cAMP response element modulator (CREM), G-protein coupled receptor 4 (GPR4), G-protein coupled receptor 31 (GPR31), G-protein coupled receptor 68 (GPR68), G-protein coupled receptor 81 (GPR81), G-protein coupled receptor 132 (GPR132), G-protein coupled receptor 151 (GPR151), inducible cAMP early repressor (ICER), and/or cyclic AMP-responsive element-binding protein 1 (CREB1) gene of the cell.
Aspect 2 is the cell of aspect 1, wherein the mutation is a partial or complete loss of function, and/or knock-out (KO) mutation.
Aspect 3 is the cell of aspect 1 or 2, wherein the mutation reduces or inhibits transcription or post-transcriptional processing of one or more mRNA isoforms encoded by the mutated endogenous gene relative to a non-mutated locus encoding the same endogenous gene.
Aspect 4 is the cell of aspect 1, wherein the mutation is a neomorphic or gain of function mutation.
Aspect 5 is the cell of aspect 1 or 4, wherein the mutation increases transcription or post-transcriptional processing of one or more mRNA isoforms encoded by the mutated endogenous gene relative to a non-mutated locus encoding the same endogenous gene.
Aspect 6 is the cell of any one of aspects 1-5, wherein the mutation results in a modified mRNA isoform population encoded by the mutated endogenous gene relative to a representative mRNA population encoded by a non-mutated locus encoding the same endogenous gene.
Aspect 7 is the cell of any one of aspects 1-4, wherein the mutation results in a modified protein isoform population encoded by the mutated endogenous gene relative to a representative protein population encoded by a non-mutated locus encoding the same endogenous gene.
Aspect 8 is the cell of any one of aspects 1-7, wherein the mutation comprises a knock-out (KO) mutation in the endogenous gene of the cell.
Aspect 9 is the cell of any one of aspects 1-7, wherein the mutation comprises a homozygous mutation in the endogenous gene of the cell.
Aspect 10 is the cell of any one of aspects 1-7, wherein the mutation comprises a heterozygous mutation in the endogenous gene of the cell.
Aspect 11 is the cell of any one of aspects 1-10, wherein the mutation results in improved cytotoxicity of the engineered cell in an acidic microenvironment and/or a tumor microenvironment (TME) relative to a control non-engineered cell.
Aspect 12 is the cell of any one of aspects 1-11, wherein the mutation results in improved cytotoxicity of the engineered cell in an acidic microenvironment with a pH less than or equal to about 7.0 relative to control non-engineered cell.
Aspect 13 is the cell of any one of aspects 1-12, wherein the mutation results in improved cytotoxicity of the engineered cell in an acidic microenvironment with a pH less than or equal to about 5.9 relative to a control non-engineered cell.
Aspect 14 is the cell of any one of aspects 1-13, wherein the mutation results in improved cytotoxicity of the engineered cell, optionally in an acidic microenvironment characterized by increased levels of lactic acid relative to a non-acidic microenvironment.
Aspect 15 is the cell of any one of aspects 1-14, wherein the mutation results in enhanced polyfunctionality of the engineered cell relative to a control non-engineered cell in response to stimulation by tumor cells.
Aspect 16 is the cell of aspect 15, wherein the enhanced polyfunctionality is evidenced by an increase in cytokine release in response to stimulation by tumor cells.
Aspect 17 is the cell of aspect 16, wherein the increase in cytokine release comprises an increase in interferon gamma (IFN-g), tumor necrosis factor alpha (TNF-a), and/or the degranulation marker CD107a, in response to stimulation by tumor cells.
Aspect 18 is the cell of aspect 15 or 16, wherein the increase in cytokine release comprises an increase in granulocyte-macrophage colony-stimulating factor (GMCSF), soluble CD137 (sCD137), INF-g, Granzyme A, interleukin 13 (IL-13), Granzyme B, soluble FAS cell surface death receptor (sFas), interleukin 6 (IL-6), soluble FAS cell surface death receptor ligand (sFasL), macrophage inflammatory protein-1 alpha (MIP-1a), macrophage inflammatory protein-1 beta (MIP-1b), TNF-α, and/or Perforin, in response to stimulation by tumor cells.
Aspect 19 is the cell of any one of aspects 1-18, wherein the mutation provides an enhanced activated and/or cytotoxic phenotype for the engineered cell relative to a control non-engineered cell.
Aspect 20 is the cell of any one of aspects 1-19, wherein the mutation provides an enhanced activated and/or cytotoxic phenotype for the engineered cell relative to a control non-engineered cell, wherein the enhanced activated and/or cytotoxic phenotype is associated with one or more of GSEA identified pathways: G2M checkpoint, E2F targets, P53 pathway, Mitotic spindle, MYC, MTORC1, Androgen Response, Unfolded Protein Response, Spermatogenesis, Heme Metabolism, TNFalpha signaling, Protein Secretion, Apoptosis, Oxidative Phosphorylation, DNA Repair, UV Response, and/or Estrogen Response Early.
Aspect 21 is the cell of any one of aspects 1-20, wherein the mutation leads to upregulation of G2M, E2F, MYC, MTORC1, oxidative phosphorylation, and/or TNFa signaling.
Aspect 22 is the cell of any one of aspects 1-21, wherein the mutation provides an enhanced proliferative capacity and/or persistence phenotype for the engineered cell relative to a control non-engineered cell.
Aspect 23 is the cell of aspect 22, wherein the enhanced proliferative capacity and/or persistence occurs in the absence of stimulation by exogenous interleukin 2 (IL-2).
Aspect 24 is the cell of aspect 22 or 23, wherein the enhanced proliferative capacity and/or persistence does not result in autonomous growth.
Aspect 25 is the cell of any one of aspects 1-24, wherein the mutation provides an enhanced metabolic fitness phenotype for the engineered cell relative to a control non-engineered cell.
Aspect 26 is the cell of aspect 25, wherein the enhanced metabolic fitness is a higher glycolytic capacity and/or improved oxygen consumption rate (OCR).
Aspect 27 is the cell of any one of aspects 1-26, wherein the mutation provides an increased chromatic accessibility across the genome when the cells are comprised in an acidic microenvironment.
Aspect 28 is the cell of any one of aspects 1-27, wherein the endogenous gene is CREM.
Aspect 29 is the cell of aspect 28, wherein the CREM mutation results in a decrease in expression of CREM RNA isoforms CREM-228 (ICER), CREM-207, CREM-230, CREM-211, CREM-213, CREM-239, CREM-201, CREM-232, CREM-217, and/or CREM-225.
Aspect 30 is the cell of aspect 28 or 29, wherein the CREM mutation results in an increase in expression of CREM RNA isoform CREM-218.
Aspect 31 is the cell of any one of aspects 28-30, wherein the CREM mutation is a result of exposure of the cell to a polynucleotide comprising the sequence of SEQ ID NO: 140 and/or SEQ ID NO: 142.
Aspect 32 is the cell of any one of aspects 28-31, wherein the CREM mutation results in a decrease in one or more CREM protein isoforms by greater than 60%.
Aspect 33 is the cell of any one of aspects 28-32, wherein the CREM mutation results in a decrease in one or more CREM protein isoforms by greater than 80%.
Aspect 34 is the cell of any one of aspects 1-33, wherein the cell has further been conditioned to acidic microenvironments by contacting the cell ex vivo with acidic stimuli.
Aspect 35 is the cell of aspect 34, wherein the acidic stimuli is provided at a concentration of greater than or equal to about 2 to 3 mM, optionally greater than or equal to about 2.5 mM.
Aspect 36 is the cell of any one of aspects 34-35, wherein the conditioning to acidic microenvironments is by incremental and/or cumulative contacting with acidic stimuli.
Aspect 37 is the cell of any one of aspects 34-36, wherein the cells are conditioned over a period of at least about 10 to 18 days, optionally at least about 14 days.
Aspect 38 is the cell of aspect 36 or 37, wherein cell conditioning comprises addition of acidic stimuli about every 48-72 hours, optionally about every 48 hours.
Aspect 39 is the cell of any one of aspects 34-37, wherein the acidic stimuli comprises or consists essentially of lactic acid.
Aspect 40 is the cell of any one of aspects 34-39, wherein the cell is conditioned to acidic microenvironments of less than or equal to about pH 6.0.
Aspect 41 is the cell of any one of aspects 1-40, wherein the cell is a T cell, natural killer (NK) cell, NK T cell, macrophage, B cell, invariant NKT cells, gamma delta T cells, MSCs, tumor-infiltrating lymphocyte, or dendritic cell.
Aspect 42 is the cell of any one of aspects 1-41, wherein the cell is a NK cell derived from cord blood (CB), peripheral blood (PB), an NK cell line, bone marrow, a stem cell, or a mixture thereof.
Aspect 43 is the cell of aspect 41 or 42, wherein the NK cell is derived from cord blood.
Aspect 44 is the cell of any one of aspects 1-43, wherein the cell comprises one or more engineered receptors.
Aspect 45 is the cell of aspect 44, wherein the one or more engineered receptors comprises an engineered antigen receptor that specifically targets an antigen.
Aspect 46 is the cell of aspect 45, wherein the engineered antigen receptor is a chimeric antigen receptor (CAR) and/or a T cell receptor (TCR).
Aspect 47 is the cell of aspect 46, wherein the engineered antigen receptor is a CAR.
Aspect 48 is the cell of any one of aspects 45-47, wherein the antigen is a cancer antigen.
Aspect 49 is the cell of any one of aspects 45-48, wherein the antigen is a solid tumor antigen.
Aspect 50 is the cell of any one of aspects 45-49, wherein the antigen is selected from the group consisting of 5T4, 8H9, αvβ6 integrin, BCMA, B7-H3, B7-H6, CAIX, CA9, CD5, CD19, CD20, CD22, CD30, CD33, CD38, CD44, CD44v6, CD44v7/8, CD70, CD123, CD138, CD171, CEA, CSPG4, CS1, CLL1, CD99, DLL3, EGFR, EGFR family including ErbB2 (HER2), EGFRVIII, EGP2, EGP40, ERBB3, ERBB4, ErbB3/4, EPCAM, EphA2, EpCAM, FAP, FBP, fetal AchR, FRα, GD2, GD3, Glypican-3 (GPC3), HLA-A1+MAGE1, HLA-A1+NY-ESO-1, IL-11Rα, IL-13Rα2, Lambda, Lewis-Y, LICAM, Kappa, KDR, MCSP, Mesothelin, Muc1, Muc16, NCAM, NKG2D Ligands, NY-ESO-1, PRAME, PSC1, PSCA, PSMA, ROR1, SP17, Survivin, TAG72, TROP2, TEMs, HMW-MAA, VEGFR2, and a combination thereof.
Aspect 51 is the cell of any one of aspects 45-50, wherein the antigen comprises TROP2 and/or CD70.
Aspect 52 is the cell of any one of aspects 44-51, wherein the one or more engineered receptors comprises a cytokine receptor, chemokine receptor, homing receptor, or a combination thereof.
Aspect 53 is the cell of any one of aspects 1-52, wherein the cell comprises expression of one or more exogenous chemokines and/or one or more cytokines.
Aspect 54 is the cell of aspect 53, wherein the cytokine is IL-15, IL-12, IL-21, IL-2, IL-18, IL-7, or a combination thereof.
Aspect 55 is the cell of aspect 54, wherein the cytokine is IL-15.
Aspect 56 is the cell of any one of aspects 1-55, wherein the cell comprises a suicide gene.
Aspect 57 is the cell of any one of aspects 1-56, wherein the endogenous gene is mutated as a result of homologous recombination or non-homologous recombination.
Aspect 58 is the cell of any one of aspects 1-57, wherein the endogenous gene is mutated by an endonuclease.
Aspect 59 is the cell of aspect 58, wherein the endonuclease is an RNA guided endonuclease.
Aspect 60 is the cell of aspect 59, wherein the RNA guided endonuclease is CRISPR-Cas9.
Aspect 61 is the cell of any one of aspects 1-60, wherein the cell comprises one or more additional mutations in one or more genes, wherein the gene is selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD38, CD40, IL21R, ICAM1, CD95, CD80, CD86, IL10R, CD5, GR, and CD7.
Aspect 62 is a population of cells according to any one of aspects 1-61.
Aspect 63 is the population of cells of aspect 62, wherein the population is comprised in a pharmaceutically acceptable excipient.
Aspect 64 is a method of treating cancer in an individual in need thereof, comprising the step of administering a therapeutically effective amount of the population of cells of aspect 62 or 63 to the individual.
Aspect 65 is the method of aspect 64, wherein the cells are autologous, allogeneic, or xenogeneic with respect to the individual.
Aspect 66 is the method of aspect 64 or 65, wherein the cells are allogeneic with respect to the individual.
Aspect 67 is the method of any one of aspects 64-66, wherein the cancer comprises a solid tumor.
Aspect 68 is the method of any one of aspects 64-66, wherein the cancer does not comprise a solid tumor.
Aspect 69 is the method of any one of aspects 64-68, wherein the cancer is of the lung, brain, breast, blood, skin, pancreas, liver, colon, head and neck, kidney, thyroid, stomach, spleen, gallbladder, bone, ovary, testes, endometrium, prostate, rectum, anus, and/or cervix.
Aspect 70 is the method of any one of aspects 64-69, wherein the individual is a mammal.
Aspect 71 is the method of aspect 70, wherein the individual is a human, dog, cat, horse, cow, sheep, pig, or rodent.
Aspect 72 is the method of aspect 70 or 71, wherein the individual is a human.
Aspect 73 is the method of any one of aspects 64-72, wherein the individual is administered an additional cancer therapy.
Aspect 74 is the method of aspect 73, wherein the additional cancer therapy is surgery, radiation, chemotherapy, hormone therapy, immunotherapy, or a combination thereof.
Aspect 75 is the method of any one of aspects 64-74, further comprising the step of diagnosing cancer in the individual.
Aspect 76 is a method of engineering an immune effector cell, comprising mutating an endogenous cAMP response element modulator (CREM), G-protein coupled receptor 4 (GPR4), G-protein coupled receptor 31 (GPR31), G-protein coupled receptor 68 (GPR68), G-protein coupled receptor 81 (GPR81), G-protein coupled receptor 132 (GPR132), G-protein coupled receptor 151 (GPR151), inducible cAMP early repressor (ICER), and/or cyclic AMP-responsive element-binding protein 1 (CREB1) gene of the cell.
Aspect 77 is the method of aspect 76, wherein the mutating generates a partial or complete loss of function, and/or knock-out (KO) mutation.
Aspect 78 is the method of aspect 76 or 77, wherein the mutating reduces or inhibits transcription or post-transcriptional processing of one or more mRNA isoforms encoded by the mutated endogenous gene relative to a non-mutated locus encoding the same endogenous gene.
Aspect 79 is the method of aspect 76, wherein the mutating generates a neomorphic or gain of function mutation.
Aspect 80 is the method of aspect 76 or 79, wherein the mutating increases transcription or post-transcriptional processing of one or more mRNA isoforms encoded by the mutated endogenous gene relative to a non-mutated locus encoding the same endogenous gene.
Aspect 81 is the method of any one of aspects 76-80, wherein the mutating generates a modified mRNA isoform population encoded by the mutated endogenous gene relative to a representative mRNA population encoded by a non-mutated locus encoding the same endogenous gene.
Aspect 82 is the method of any one of aspects 76-79, wherein the mutating generates a modified protein isoform population encoded by the mutated endogenous gene relative to a representative protein population encoded by a non-mutated locus encoding the same endogenous gene.
Aspect 83 is the method of any one of aspects 76-82, wherein the mutating generates a knock-out (KO) mutation in the endogenous gene of the cell.
Aspect 84 is the method of any one of aspects 76-82, wherein the mutating generates a homozygous mutation in the endogenous gene of the cell.
Aspect 85 is the method of any one of aspects 76-82, wherein the mutating generates a heterozygous mutation in the endogenous gene of the cell.
Aspect 86 is the method of any one of aspects 76-85, wherein the mutating generates an improved cytotoxicity of the engineered cell in an acidic microenvironment and/or a tumor microenvironment (TME) relative to a control non-engineered cell.
Aspect 87 is the method of any one of aspects 76-86, wherein the mutating generates an improved cytotoxicity of the engineered cell in an acidic microenvironment with a pH less than or equal to about 7.0 relative to control non-engineered cell.
Aspect 88 is the method of any one of aspects 76-87, wherein the mutating generates an improved cytotoxicity of the engineered cell in an acidic microenvironment with a pH less than or equal to about 5.9 relative to a control non-engineered cell.
Aspect 89 is the method of any one of aspects 76-88, wherein the mutating results in improved cytotoxicity of the engineered cell in an acidic microenvironment characterized by increased levels of lactic acid relative to a non-acidic microenvironment.
Aspect 90 is the method of any one of aspects 76-89, wherein the mutating generates an enhanced polyfunctionality of the engineered cell relative to a control non-engineered cell in response to stimulation by tumor cells.
Aspect 91 is the method of aspect 90, wherein the enhanced polyfunctionality is evidenced by an increase in cytokine release in response to stimulation by tumor cells.
Aspect 92 is the method of aspect 91, wherein the increase in cytokine release comprises an increase in interferon gamma (IFN-g), tumor necrosis factor alpha (TNF-a), and/or the degranulation marker CD107a, in response to stimulation by tumor cells.
Aspect 93 is the method of aspect 90 or 91, wherein the increase in cytokine release comprises an increase in granulocyte-macrophage colony-stimulating factor (GMCSF), soluble CD137 (sCD137), INF-g, Granzyme A, interleukin 13 (IL-13), Granzyme B, soluble FAS cell surface death receptor (sFas), interleukin 6 (IL-6), soluble FAS cell surface death receptor ligand (sFasL), macrophage inflammatory protein-1 alpha (MIP-1a), macrophage inflammatory protein-1 beta (MIP-1b), TNF-α, and/or Perforin, in response to stimulation by tumor cells.
Aspect 94 is the method of any one of aspects 76-93, wherein the mutating generates an enhanced activated and/or cytotoxic phenotype for the engineered cell relative to a control non-engineered cell.
Aspect 95 is the method of any one of aspects 76-93, wherein the mutating leads to an enhanced activated and/or cytotoxic phenotype for the engineered cell relative to a control non-engineered cell, wherein the enhanced activated and/or cytotoxic phenotype is associated with one or more of GSEA identified pathways: G2M checkpoint, E2F targets, P53 pathway, Mitotic spindle, MYC, MTORC1, Androgen Response, Unfolded Protein Response, Spermatogenesis, Heme Metabolism, TNFalpha signaling, Protein Secretion, Apoptosis, Oxidative Phosphorylation, DNA Repair, UV Response, and/or Estrogen Response Early.
Aspect 96 is the method of any one of aspects 76-95, wherein the mutating leads to upregulation of G2M, E2F, MYC, MTORC1, oxidative phosphorylation, and/or TNFa signaling.
Aspect 97 is the method of any one of aspects 76-96, wherein the mutating generates an enhanced proliferative capacity and/or persistence phenotype for the engineered cell relative to a control non-engineered cell.
Aspect 98 is the method of aspect 97, wherein the enhanced proliferative capacity and/or persistence occurs in the absence of stimulation by exogenous interleukin 2 (IL-2).
Aspect 99 is the method of aspect 97 or 98, wherein the enhanced proliferative capacity and/or persistence does not result in autonomous growth.
Aspect 100 is the method of any one of aspects 76-99, wherein the mutating generates an enhanced metabolic fitness phenotype for the engineered cell relative to a control non-engineered cell.
Aspect 101 is the method of aspect 100, wherein the enhanced metabolic fitness is a higher glycolytic capacity and/or improved oxygen consumption rate (OCR).
Aspect 102 is the method of any one of aspects 76-101, wherein the mutating provides an increased chromatic accessibility across the genome when the cells are comprised in an acidic microenvironment.
Aspect 103 is the method of any one of aspects 76-102, wherein the endogenous gene is CREM.
Aspect 104 is the method of aspect 103, wherein the mutating of CREM results in a decrease in expression of CREM RNA isoforms CREM-228 (ICER), CREM-207, CREM-230, CREM-211, CREM-213, CREM-239, CREM-201, CREM-232, CREM-217, and/or CREM-225.
Aspect 105 is the method of aspect 103 or 104, wherein the mutating of CREM results in an increase in expression of CREM RNA isoform CREM-218.
Aspect 106 is the method of any one of aspects 103-105, wherein the mutating of CREM comprises exposure of the cell to a polynucleotide comprising the sequence of SEQ ID NO: 140 and/or SEQ ID NO: 142.
Aspect 107 is the method of any one of aspects 103-106, wherein the mutating of CREM generates a decrease in one or more CREM protein isoforms by greater than 60%.
Aspect 108 is the method of any one of aspects 103-107, wherein the mutating of CREM generates a decrease in one or more CREM protein isoforms by greater than 80%.
Aspect 109 is the method of any one of aspects 76-108, further comprising conditioning the cell to acidic microenvironments by contacting the cell ex vivo with acidic stimuli.
Aspect 110 is the method of aspect 109, comprising providing the acidic stimuli at a concentration of greater than or equal to about 2 to 3 mM, optionally greater than or equal to about 2.5 mM.
Aspect 111 is the method of aspect 109 or 110, wherein the conditioning to acidic microenvironments is by incremental and/or cumulative contacting with acidic stimuli.
Aspect 112 is the method of any one of aspects 109-111, wherein the conditioning is over a period of at least about 10 to 18 days, optionally at least about 14 days.
Aspect 113 is the method of any one of aspects 109-112, wherein the conditioning comprises addition of acidic stimuli about every 48-72 hours, optionally about every 48 hours.
Aspect 114 is the method of any one of aspects 109-113, wherein the acidic stimuli comprises or consists essentially of lactic acid.
Aspect 115 is the method of any one of aspects 109-114, wherein the conditioning is to acidic microenvironments of less than or equal to about pH 6.0.
Aspect 116 is the method of any one of aspects 76-115, wherein the cell is a T cell, natural killer (NK) cell, NK T cell, macrophage, B cell, invariant NKT cells, gamma delta T cells, MSCs, tumor-infiltrating lymphocyte, or dendritic cell.
Aspect 117 is the method of any one of aspects 76-116, wherein the cell is a NK cell derived from cord blood (CB), peripheral blood (PB), an NK cell line, bone marrow, a stem cell, or a mixture thereof.
Aspect 118 is the method of aspect 116 or 117, wherein the NK cell is derived from cord blood.
Aspect 119 is the method of any one of aspects 76-118, wherein the cell comprises one or more engineered receptors that specifically binds an antigen.
Aspect 120 is the method of aspect 119, wherein the one or more engineered receptors comprises an engineered antigen receptor.
Aspect 121 is the method of aspect 120, wherein the engineered antigen receptor is a chimeric antigen receptor (CAR) and/or a T cell receptor (TCR).
Aspect 122 is the method of aspect 121, wherein the engineered antigen receptor is a CAR.
Aspect 123 is the method of any one of aspects 120-122, wherein the antigen is a cancer antigen.
Aspect 124 is the method of any one of aspects 120-123, wherein the antigen is a solid tumor antigen.
Aspect 125 is the method of any one of aspects 120-124, wherein the antigen is selected from the group consisting of 5T4, 8H9, αvβ6 integrin, BCMA, B7-H3, B7-H6, CAIX, CA9, CD5, CD19, CD20, CD22, CD30, CD33, CD38, CD44, CD44v6, CD44v7/8, CD70, CD123, CD138, CD171, CEA, CSPG4, CS1, CLL1, CD99, DLL3, EGFR, EGFR family including ErbB2 (HER2), EGFRVIII, EGP2, EGP40, ERBB3, ERBB4, ErbB3/4, EPCAM, EphA2, EpCAM, FAP, FBP, fetal AchR, FRα, GD2, GD3, Glypican-3 (GPC3), HLA-A1+MAGE1, HLA-A1+NY-ESO-1, IL-11Rα, IL-13Rα2, Lambda, Lewis-Y, LICAM, Kappa, KDR, MCSP, Mesothelin, Muc1, Muc16, NCAM, NKG2D Ligands, NY-ESO-1, PRAME, PSC1, PSCA, PSMA, ROR1, SP17, Survivin, TAG72, TROP2, TEMs, HMW-MAA, VEGFR2, and a combination thereof.
Aspect 126 is the method of any one of aspects 120-125, wherein the antigen comprises TROP2 and/or CD70.
Aspect 127 is the method of any one of aspects 119-126, wherein the one or more engineered receptors comprises a cytokine receptor, chemokine receptor, homing receptor, or a combination thereof.
Aspect 128 is the method of any one of aspects 76-127, wherein the cell comprises expression of one or more exogenous chemokines and/or one or more cytokines.
Aspect 129 is the method of aspect 128, wherein the cytokine is IL-15, IL-12, IL-21, IL-2, IL-18, IL-7, or a combination thereof.
Aspect 130 is the method of aspect 129, wherein the cytokine is IL-15.
Aspect 131 is the method of any one of aspects 76-130, wherein the cell comprises a suicide gene.
Aspect 132 is the method of any one of aspects 76-131, wherein the mutating of the endogenous gene is comprises homologous recombination or non-homologous recombination.
Aspect 133 is the method of any one of aspects 76-132, wherein the mutating of the endogenous gene is mediated by an endonuclease.
Aspect 134 is the method of aspect 133, wherein the endonuclease is an RNA guided endonuclease.
Aspect 135 is the method of aspect 134, wherein the RNA guided endonuclease is CRISPR-Cas9.
Aspect 136 is the method of any one of aspects 76-135, wherein the cell comprises one or more additional mutations in one or more genes, wherein the gene is selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD38, CD40, IL21R, ICAM1, CD95, CD80, CD86, IL10R, CD5, GR, and CD7.
Aspect 137 is an engineered natural killer (NK) cell, wherein the cell comprises an engineered partial or complete loss of function, and/or knock-out (KO) mutation in an endogenous cAMP response element modulator (CREM) gene in the cell.
Aspect 138 is the cell of aspect 137, wherein the cell comprises a knock-out (KO) mutation in the endogenous CREM gene in the cell.
Aspect 139 is the cell of aspect 138, wherein the cell comprises a homozygous knock-out (KO) mutation in the endogenous CREM gene.
Aspect 140 is the cell of aspect 138, wherein the cell comprises heterozygous KO mutation in the endogenous CREM gene in the cell.
Aspect 141 is the cell of aspect 137, wherein the mutation results in improved cytotoxicity of the engineered cell relative to a reference cell lacking the mutation.
Aspect 142 is the cell of aspect 137, wherein the mutation results in improved cytotoxicity of the engineered cell in an acidic microenvironment and/or a tumor microenvironment (TME) relative to a control non-engineered cell.
Aspect 143 is the cell of aspect 137, wherein the mutation results in improved cytotoxicity of the engineered cell in an acidic microenvironment with a pH less than or equal to about 7.0 relative to control non-engineered cell.
Aspect 144 is the cell of aspect 137, wherein the mutation results in improved cytotoxicity of the engineered cell in an acidic microenvironment with a pH less than or equal to about 5.9 relative to a control non-engineered cell.
Aspect 145 is the cell of aspect 137, wherein the mutation results in improved cytotoxicity of the engineered cell in an acidic environment characterized by increased levels of lactic acid relative to a non-acidic microenvironment.
Aspect 146 is the cell of aspect 137, wherein the mutation results in enhanced polyfunctionality of the engineered cell relative to a control non-engineered cell in response to stimulation by tumor cells.
Aspect 147 is the cell of aspect 146, wherein the enhanced polyfunctionality is evidenced by an increase in cytokine release in response to stimulation by tumor cells.
Aspect 148 is the cell of aspect 137, wherein the increase in cytokine release comprises an increase in interferon gamma (IFN-g), tumor necrosis factor alpha (TNF-a), and/or the degranulation marker CD107a, in response to stimulation by tumor cells.
Aspect 149 is the cell of aspect 148, wherein the increase in cytokine release comprises an increase in granulocyte-macrophage colony-stimulating factor (GMCSF), soluble CD137 (sCD137), INF-g, Granzyme A, interleukin 13 (IL-13), Granzyme B, soluble FAS cell surface death receptor (sFas), interleukin 6 (IL-6), soluble FAS cell surface death receptor ligand (sFasL), macrophage inflammatory protein-1 alpha (MIP-1a), macrophage inflammatory protein-1 beta (MIP-1b), TNF-α, and/or Perforin, in response to stimulation by tumor cells.
Aspect 150 is the cell of aspect 137, wherein the mutation provides an enhanced activated and/or cytotoxic phenotype for the engineered cell relative to a control non-engineered cell.
Aspect 151 is the cell of aspect 150, wherein the mutation provides an enhanced activated and/or cytotoxic phenotype for the engineered cell relative to a control non-engineered cell, wherein the enhanced activated and/or cytotoxic phenotype is associated with one or more of GSEA identified pathways: G2M checkpoint, E2F targets, P53 pathway, Mitotic spindle, MYC, MTORC1, Androgen Response, Unfolded Protein Response, Spermatogenesis, Heme Metabolism, TNFalpha signaling, Protein Secretion, Apoptosis, Oxidative Phosphorylation, DNA Repair, UV Response, and/or Estrogen Response Early.
Aspect 152 is the cell of aspect 137, wherein the mutation provides for upregulation of G2M, E2F, MYC, MTORC1, oxidative phosphorylation, and/or TNFa signaling.
Aspect 153 is the cell of aspect 137, wherein the mutation provides an enhanced proliferative capacity and/or persistence phenotype for the engineered cell relative to a control non-engineered cell.
Aspect 154 is the cell of aspect 153, wherein the enhanced proliferative capacity and/or persistence occurs in the absence of stimulation by exogenous interleukin 2 (IL-2).
Aspect 155 is the cell of aspect 153 or 154, wherein the enhanced proliferative capacity and/or persistence does not result in autonomous growth.
Aspect 156 is the cell of aspect 137, wherein the mutation provides an enhanced metabolic fitness phenotype for the engineered cell relative to a control non-engineered cell.
Aspect 157 is the cell of aspect 137, wherein the enhanced metabolic fitness is a higher glycolytic capacity and/or improved oxygen consumption rate (OCR).
Aspect 158 is the cell of aspect 137, wherein the mutation provides an increased chromatic accessibility across the genome when the cells are comprised in an acidic environment.
Aspect 159 is the cell of aspect 137, wherein the CREM mutation results in a decrease in expression of CREM RNA isoforms CREM-228 (ICER), CREM-207, CREM-230, CREM-211, CREM-213, CREM-239, CREM-201, CREM-232, CREM-217, and/or CREM-225
Aspect 160 is the cell of aspect 137, wherein the CREM mutation is a result of exposure of the cell to a polynucleotide comprising the sequence of SEQ ID NO: 140 and/or SEQ ID NO: 142.
Aspect 161 is the cell of aspect 137, wherein the CREM mutation results in a decrease in CREM protein isoforms by greater than 60%; or wherein the CREM mutation results in a decrease in CREM protein isoforms by greater than 80%.
Aspect 162 is the cell of aspect 137, wherein the cell is derived from cord blood (CB).
Aspect 163 is the cell of aspect 137, wherein the cell is derived from peripheral blood (PB).
Aspect 164 is the cell of aspect 137, wherein the cell is derived from a stem cell.
Aspect 165 is the cell of aspect 137, wherein the cells comprises an engineered receptor.
Aspect 166 is the cell of aspect 137, wherein the cell comprises a T cell receptor (TCR).
Aspect 167 is the cell of aspect 137, wherein the cell comprises a chimeric antigen receptor (CAR).
Aspect 168 is the cell of any one of aspects 165-167, wherein the engineered receptor specifically binds an antigen selected from the group consisting of 5T4, 8H9, αvβ6 integrin, BCMA, B7-H3, B7-H6, CAIX, CA9, CD5, CD19, CD20, CD22, CD30, CD33, CD38, CD44, CD44v6, CD44v7/8, CD70, CD123, CD138, CD171, CEA, CSPG4, CS1, CLL1, CD99, DLL3, EGFR, EGFR family including ErbB2 (HER2), EGFRVIII, EGP2, EGP40, ERBB3, ERBB4, ErbB3/4, EPCAM, EphA2, EpCAM, FAP, FBP, fetal AchR, FRα, GD2, GD3, Glypican-3 (GPC3), HLA-A1+MAGE1, HLA-A1+NY-ESO-1, IL-11Rx, IL-13Rα2, Lambda, Lewis-Y, LICAM, Kappa, KDR, MCSP, Mesothelin, Muc1, Muc16, NCAM, NKG2D Ligands, NY-ESO-1, PRAME, PSC1, PSCA, PSMA, ROR1, SP17, Survivin, TAG72, TROP2, TEMs, HMW-MAA, VEGFR2, and a combination thereof.
Aspect 169 is the cell of aspect 168, wherein the antigen is TROP2.
Aspect 170 is the cell of aspect 168, wherein the antigen is CD70.
Aspect 171 is a population of cells according to any one of aspects 137-170
Aspect 172 is a pharmaceutical composition comprising the cell of any one of aspect 137-170, and optionally a pharmaceutically acceptable excipient.
Aspect 173 is a method of treating cancer in an individual in need thereof, comprising the step of administering a therapeutically effective amount of the cell of any one of aspects 137-170, the population of aspect 171, or the pharmaceutical composition of aspect 172 to the individual.
Aspect 174 is the method of aspect 173, wherein the cancer comprises a solid tumor.
Aspect 175 is the method of aspect 173, wherein the cancer is of the lung, brain, breast, blood, skin, pancreas, liver, colon, head and neck, kidney, thyroid, stomach, spleen, gallbladder, bone, ovary, testes, endometrium, prostate, rectum, anus, and/or cervix.
Aspect 176 is the method of aspect 173, wherein the individual is a mammal.
Aspect 177 is the method of aspect 173, wherein the individual is a human.
Aspect 178 is a method of engineering a cell according to any one of aspects 137-170.
Aspect 179 is a method of killing a cancer cell, comprising contacting the cancer cell with an engineered NK cell according to any one of aspects 137-170.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Brief Summary, Detailed Description, Claims, Abstract, and Brief Description of the Drawings.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-B. Differential upregulation of genes involved in acidity signaling in tumor-infiltrating NK cells in a highly glycolytic tumor. (1A) High relative expression of genes involved in enzymatic reactions of glycolysis leading to lactic acid and accumulation of other acids in the tumor microenvironment in primary pancreatic cancer tissues analyzed through single cell RNA sequencing. (1B) Pancreatic cancer-infiltrating natural killer (NK) cells showed an upregulation of a number of proton-sensing G-protein coupled receptors (GPR) such as GPR68, and GPR132, as well as downstream signaling mediators of response to acidity in the cyclic AMP (CAMP) pathway such as CAMP Responsive Element Modulator (CREM) and CAMP Responsive Element Binding Protein 1 (CREB1).

FIGS. 2A-C. Upregulation of CREM expression in tumor-infiltrating immune cells of a highly glycolytic tumor. (2A) High relative expression of genes involved in enzymatic reactions of glycolysis leading to accumulation of lactic acid and other acids in the tumor microenvironment of clear cell renal cell carcinoma was revealed by analysis of The Cancer Genome Atlas (TCGA) Program data. (2B) Low expression of CREM in peripheral blood mononuclear cells (PBMC) from healthy donors was revealed by analysis of single cell RNA sequencing data available in the Tumor Immune Single-cell Hub (TISCH) database. (C) High relative expression of CREM in renal cell carcinoma-infiltrating immune cells was revealed by analysis of single cell RNA sequencing data available in the TISCH database.

FIGS. 3A-B. High knock-out (KO) efficiency of CREM using CRISPR-Cas9. (3A) Confirmation of CREM knock-out (KO) by PCR of DNA obtained from CAR-NK cells with CREM wild-type (WT) loci vs. CREM KO loci. 1 Kb Plus DNA Ladder (Invitrogen) was used. The DNA band amplified from CREM WT NK cells falls on 300 bp band while the one from CREM KO NK cells falls around 200 bd band (3B) Analysis of selected CREM isoforms from bulk RNA sequencing data obtained from CREM WT vs. CREM KO NK cells derived from 3 cord blood donors (D1, D2, and D3). The heatmap color scale is representative of the counts of expressing cells from 0 (dark blue) to 400 (red); the FDR column corresponds to the false discovery rate (significance <0.05); while the logPostFC column represents the log of the fold change of WT vs. KO expression levels, positive values indicated that expression is higher in WT and negative values indicated that expression is higher in KO cells. The isoform expression analysis demonstrated that multiple CREM RNA isoforms were being significantly knocked out in NK cells. Examples included: the ICER isoform (CREM-228), CREM-207, CREM-211, CREM-213, CREM-239, CREM-201, CREM-232, and CREM-217.

FIGS. 4A-D. CREM KO significantly enhanced NK cell cytotoxicity against highly glycolytic tumor models. (4A) Photographs representative of NK cell cytotoxicity against the highly glycolytic UMRC3 cell line expressing green fluorescent protein (GFP) at 0 hours (T=0) and after 48 hours (T=48 h) of coculture with either CREM WT or CREM KO CAR-NK cells. (4B) Depicts quantification of GFP+UMRC3 tumor growth over time in a long-term cytotoxicity assay using INCUCYTE®. (4C) Photographs representative of NK cell cytotoxicity against highly glycolytic GFP+UMRC3 cell line grown into spheroids (3D tumor masses) at T=42 h (when spheroids have formed) and 48 h (when NK cells were added; (T=48 h)) as well as 7 days (T=7 d) later. (4D) Depicts quantification of GFP+UMRC3 spheroid growth over time in a long-term cytotoxicity assay using INCUCYTER.

FIGS. 5A-B. CREM KO overcomes NK cell cytotoxicity suppression under an acidic condition. (5A) Depicts the cytotoxicity of NK cells against kidney cancer cells (e.g., A498 cell line) at a typical culture medium pH (pH=7.2) when compared to an acidic culture medium pH (pH=5.9) as determined by Chromium release assay at effector to target (E:T) ratios of 20:1, 10:1, 5:1, 2.5:1, and 1.25:1. Multiple paired t-tests were used to compare cytotoxicity at the 2 pHs at each E:T ratio. (5B) Shows a comparison of the cytotoxicity of CREM WT NK cells vs. CREM KO NK cells at pH 7.2 or pH 5.9. One-way Anova with multiple comparison using the corrected method of Benjamini and Yekutieli was used to compare the overall cytotoxicity of CREM WT NK cells vs. CREM KO NK cells.

FIGS. 6A-C. CREM KO CAR-NK cells displayed enhanced polyfunctionality as evident by an increase in cytokine release. (6A) Displays FACS plots representative of release of cytokines (INF-gamma (INF-y or IFN-g) and TNF-alpha (TNF-α or TNF-a) as well as the degranulation marker CD107a in NT NK, CREM WT CAR-NK, or CREM KO CAR-NK cells in response to stimulation by glycolytic tumor cells SKOV3 followed by intracellular staining. (6B) Shows a summary of INF-g, TNF-α, and CD107a percentage release across the NK cell conditions: non-transduced (NT) NK cells, CREM WT CAR-NK cells, and CREM KO CAR-NK cells. (6C) Displays a heatmap of various cytokine levels released by NT NK cells, CREM WT CAR-NK cells, or CREM KO CAR-NK cells when assayed by LUMINEX® (multiplex assay of analytes shown). Each column shows the mean level of a marker normalized to the condition with the highest expression level across the test conditions (e.g., generally CREM KO CAR-NK), expression is shown as a range of 0 (blue) to 1 (yellow).

FIG. 7. CREM KO CAR-NK cells have an immunophenotype characterized by activation and cytotoxicity. Is a heatmap showing the mass cytometry proteomic marker level (by Cytometry by Time-of-Flight (CyTOF)) of non-transduced (NT) NK cells, CREM WT CAR-NK cells, and CREM KO CAR-NK cells. Columns correspond to the various NK cell conditions. Each row shows the mean level of a marker normalized over columns to a range of 0 (blue) to 1 (yellow).

FIG. 8. CREM KO enhances the proliferation capacity and persistence of CAR-NK cells in the absence of stimulation by IL-2 but does not lead to autonomous growth. Displays NK cell counts following cultures of non-transduced (NT) NK cells, CREM WT CAR-NK cells, and CREM KO CAR-NK cells (x10⁶) without stimulation by IL-2, over time in days.

FIGS. 9A-B. CREM KO enhances metabolic fitness of CAR-NK cells. (9A) Shows extracellular acidification rate (ECAR) measurements determined by SEAHORSE® glycolysis stress test assays as a representation of aerobic glycolysis of non-transduced (NT) NK cells, CREM WT CAR-NK cells, and CREM KO CAR-NK cells. (9B) Oxygen consumption rate (OCR) measurements determined by SEAHORSE® mito stress test assays as a representation of oxidative phosphorylation for the various NK cell conditions.

FIGS. 10A-H. CREM KO enabled CAR-NK cells to withstand the suppressive effect of lactic acid at concentrations similar to those observed in tumor microenvironments (TME). (10A) Depicts the results of a cytotoxicity assay (e.g., 51Cr release assay) of anti-CD70 CAR-NK (CAR.70) cells against renal cell carcinoma (e.g., UMRC3 renal cell carcinoma tumor cells) at various E:T ratios (e.g., 20:1, 10:1, 5:1, 2.5: and 1.25:1 respectively). NK cells were either CREM wild-type (WT) or CREM knock-out (KO), and were incubated in either a regular pH (pH^{Reg}) or an acidic pH (pH^{Lac}) (e.g., a pH of approximately 6) created using lactic acid. (10B) Shows representative brightfield microscopy pictures of CAR-NK cells and UMRC3 tumor cells (elongated cells) cocultured at a regular pH (pH^{Reg}) or an acidic pH (pH^{Lac}). The cell clumps indicated areas of active engagement of cytotoxic CAR-NK cells with the tumor cells. (10C) Depicts results of a representative long-term cytotoxicity (e.g., Incucyte®) assay of CAR.70 NK cells and their non-transduced (NT) counterparts in pH^{Reg} or pH^{Lac} environments when challenged against UMRC3 cells. NK cells were conditioned to incrementally (Inc) more acidic conditions (e.g., by adding approximately 2.5 mM of lactic acid every 2-3 days) over a 2-week incubation period (pH^{Lac20(Inc)} results in a pH of approximately 6.0) or maintained in a pH^{Reg} throughout the same period. All conditions started from the regular pH of culture media (pH^{Reg} ˜7.4). Following the 2-week conditioning period, NK cells of the various conditions were challenged with UMRC3 cells at the noted pHs (pH^{Reg} or pH^{Lac20(Inc)}). The results indicated that while chronic lactic acid exposure dampened the cytotoxicity of CAR-NK cells when compared to those in a regular pH, the CREM KO CAR-NK cells were capable of maintaining robust anti-tumor killing despite this relatively extreme condition that mimicked the low pH of the tumor microenvironment of glycolytic tumors. (10D) Displays representative Incucyte® images of NT NK cells (NT), CAR.70 CREM WT NK cells (CAR.70), or CAR.70 CREM KO NK cells challenged with GFP+UMRC3 cells at 1:1 or 2:1 E:T ratios in acidic (pH^{Lac20(Inc)}) conditions. (10E) Shows multiparametric spectral flow cytometry mediated phenotypic characterization of CAR.70 NK cells that were either CREM WT or CREM KO and cocultured with UMRC3 tumor cells under either pH^{Reg} or pH^{Lac} conditions. viSNE analysis was performed on the various conditions followed by FlowSOM clustering. (10F) Shows distribution of the 4 FlowSOM metaclusters (MCs) across the different conditions demonstrated an increase in MC2 (orange) in CREM WT CAR.70 NK cells cocultured with UMRC3 cells at pH^{Lac}. However, the CREM KO CAR.70 NK cells cocultured with UMRC3 cells in pH^{Lac} exhibited a similar composition of MC2 phenotyped cells as those NK cells cocultured in pH^{Reg} conditions. (10G) Displays expression of various markers in the 4 MCs normalized locally across conditions to minimum and maximum expression on a color scale of blue (minimum) to red (maximum). The percentage expression was overlayed by circles of various sizes proportional to the % expression. MC2 was generally characterized by a relatively low expression of activation markers and a relatively high expression of certain checkpoints (e.g., TIGIT and LAG3) and markers of terminal differentiation (e.g., CD57). (10H) Shows contour tSNE plots of the various conditions (pH^{Reg} or pH^{Lac}, and CREM WT or CREM KO) showing the gross phenotypic clustering differences in NK cell distribution following viSNE analysis.

FIGS. 11A-F. CREM KO improved CAR-NK cell activation and cytotoxicity in a long-term assay of multiple tumor rechallenges. (11A) Displays results of a representative rechallenge cytotoxicity assay using xCELLigence® of CREM WT and CREM KO anti-CD70 CAR-NK cells (CAR.70) and their non-transduced (NT) counterparts against UMRC3 tumor cells. The UMRC3 cells were plated in E-plates and allowed to adhere and grow overnight. Twenty-four hours later, the noted NK cells were added at an E:T ratio of 2:1. Every 2-4 days, new tumor cells were plated in new E-plates. Twenty-four hours later, the NK cells in the first E-plate were collected and counted and transferred at the same E:T ratio of 2:1 onto the new tumor plate. (11B) Shows phenotypic characterization of CAR.70 NK cells that were either CREM WT or CREM KO at the end of the rechallenge assay shown in (11A). viSNE analysis was performed on the various conditions followed by FlowSOM clustering into 5 FlowSOM groups (C1-C5). (11C) Displays distribution of the 5 FlowSOM metaclusters (MCs, C1-C5) for cells described in FIGS. 11A-11B, the results demonstrated an increase in MC5 and a decrease in MC4 in CREM KO CAR.70 NK cells when compared to CREM WT cells. (11D) Shows contour tSNE plots of CREM WT CAR.70 NK cells (top) and CREM KO CAR.70 NK cells (bottom) showing the gross phenotypic clustering differences in their distribution following viSNE analysis. (11E) Shows expression of the various markers in the 5 MCs (C1-C5) normalized locally across conditions to minimum and maximum expression on a color scale of blue (minimum) to orange (maximum). The percentage expression was overlayed by circles of various sizes proportional to the % expression. MC4 was generally characterized by a relatively low level of expression of activation markers, while MC5 was characterized by an opposite pattern of overall increases in markers of activation. (11F) Shows expression of select markers (ICOS, DNAM1, CD16, OX40, TIGIT, and TIM3) at single cell levels when compared between the CREM WT CAR.70 NK cells and CREM KO CAR.70 NK cells in MC4 and MC5. The results showed significant differences in marker expression in CREM KO CAR.70 NK cells relative to control CREM WT CAR.70 NK cells by Kruskal-Wallis test with Dunn's multiple comparisons.

FIGS. 12A-C. CREM KO enhanced the cytotoxicity of CAR-NK cells in multiple different tumor models and using multiple different CAR constructs while targeting multiple different tumor antigens both in vitro in 3D tumor spheroid models as well as in vivo in mouse models. (12A) Shows how CREM KO significantly enhanced cytotoxicity of TROP2-directed CAR-NK cells (CAR.TROP2) against 3D spheroids of pancreatic cancer cells (e.g., GFP+PATC148 cells) relative to CREM WT TROP-2 directed CAR-NK cells, by ordinary one-way ANOVA and Tukey's multiple comparisons test with a single pooled variance. (12B) Depicts representative bioluminescence imaging (BLI) of mouse groups injected peritoneally with TROP2+expressing ovarian cancer cells (luciferase positive SKOV3 cells) only, injected with tumor cells and CREM WT CAR.TROP2 CAR-NK cells, or injected with tumor cells and CREM KO CAR.TROP2 CAR-NK cells. Images show the burden of luciferase positive tumor cells on day 47 after NK cell injection. Six days following tumor implantation, the mice were irradiated, the following day mice were treated intraperitoneally with 4 million NK cells (CAR.TROP2 transduced NK cells that were CREM WT or CREM KO respectively). The results showed that CREM KO enhanced the in vivo cytotoxicity of CAR NK cells against solid tumors. (12C) Shows representative images (in duplicate) of cytotoxicity assays of CREM WT or CREM KO anti-CD70 (CAR.70) CAR-NK cells against CD70-positive breast cancer (e.g., breast cancer cell line BCX010) in spheroid assays conducted using Incucyte®. BCX010 (GFP-positive) cells were plated in ultra low-attachment (ULA) plates and allowed to form spheroids over 24 hours. The following day, NK cells were added to the corresponding wells. The results showed CREM KO CAR-NK cells reduced CD70+tumor cell fluorescence relative to CREM WT CAR-NK cells at 3 days following NK cell addition.

FIGS. 13A-C. CREM KO enhanced the metabolic fitness of NK cells at baseline as well as when cultured in an acidic pH. (13A) Depicts the extracellular acidification rate (ECAR; as a surrogate of glycolysis, top panel) of CREM WT or CREM KO anti-CD70 CAR-NK cells (CAR.70) as well as their non-transduced (NT) counterparts. The CAR.70 construct was designed to also secrete interleukin-15 (IL15), and control NK cells that were transduced with a construct expressing IL 15 in the absence of a CAR were included in the assay (IL15 NK cells). The assay was performed on NK cells cultured in regular media without stimulation using the Seahorse® glyco stress test assay following the manufacturer's protocol. Oxygen consumption rate (OCR; as a surrogate for oxidative phosphorylation (OXPHOS), bottom panel) was analyzed using the Seahorse® mito stress test assay following the manufacturer's protocol. (13B) Displays the ECAR (top) and OCR (bottom) of NT, CREM WT CAR-NK cells and CREM KO CAR-NK cells incubated in lactic acid. (13C) Shows how pathways of glycolysis and OXPHOS were significantly upregulated in CREM KO NK cells when compared to CREM WT NK cells following incubation with lactic acid (analyzed using unbiased GSEA performed on bulk RNA sequencing of these conditions).

FIGS. 14A-B. CREM KO NK cells displayed distinct transcriptomic and epigenetic profiles when compared to CREM WT NK cells upon incubation with lactic acid. (14A) Shows a bar graph of differentially upregulated or downregulated HALLMARK pathways in CREM KO vs. CREM WT NK cells incubated in lactic acid for 24 hours. Analysis was performed using gene set enrichment analysis (GSEA) on bulk RNA sequencing of the samples. Notably, CREM KO was associated with upregulation of pathways of proliferation (G2M, E2F), MYC, MTORC1, oxidative phosphorylation, and TNFa signaling. (14B) Displays chromatin accessibility CREM KO vs. CREM WT NK cells incubated in lactic acid for 24 hours as assessed by bulk ATAC sequencing of the corresponding samples. Each row represented one peak displayed over a 4-kb window. Three cord blood donors of NK cells were used (WT-1, WT-2, WT-3) as well as their CREM KO counterparts (KO-1, KO-2, KO-3). Notably, CREM KO was associated with a pattern of significant increase in chromatic accessibility across the genome.

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure.

The term “exogenous” as used herein refers to a polynucleotide (such as one encoding a gene product or part of a gene product) that is not present endogenously in a mammalian cell, such as an immune cell, or is synthetically generated outside of a mammalian cell, such as by recombinant technology.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. Accordingly, a “gene product” as used herein, refers to transcribed mRNA, pre-splicing transcribed RNA (for example, RNA which still comprises non-coding region), translated polypeptide (for example, those with or without signal peptide or other region not present in the mature protein), and protein. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, such as that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides that are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also include reducing the intensity, effect, symptoms and/or burden of a disease or condition prior to onset or recurrence of the disease or condition.

The term “sample,” as used herein, generally refers to a biological sample. The sample may be taken from tissue or cells from an individual. In some examples, the sample may comprise, or be derived from, a tissue biopsy, blood (e.g., whole blood), blood plasma, extracellular fluid, dried blood spots, cultured cells, discarded tissue. The sample may have been isolated from the source prior to collection. Non-limiting examples include blood, cerebral spinal fluid, pleural fluid, amniotic fluid, lymph fluid, saliva, urine, stool, tears, sweat, or mucosal excretions, and other bodily fluids isolated from the primary source prior to collection. In some examples, the sample is isolated from its primary source (cells, tissue, bodily fluids such as blood, environmental samples, etc.) during sample preparation. The sample may or may not be purified or otherwise enriched from its primary source. In some cases the primary source is homogenized prior to further processing. The sample may be filtered or centrifuged to remove buffy coat, lipids, or particulate matter. The sample may also be purified or enriched for nucleic acids, or may be treated with RNases. The sample may contain tissues or cells that are intact, fragmented, or partially degraded.

The term “subject,” as used herein, generally refers to an individual having a biological sample that is undergoing processing or analysis and, in specific cases, has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may be undergoing or having undergone treatment. The subject may be asymptomatic. The subject may be healthy individuals but that are desirous of prevention of cancer. The term “individual” is used interchangeably with “subject”. The “subject” or “individual”, as used herein, may or may not be housed in a medical facility and may be treated as an outpatient of a medical facility. The individual may be receiving one or more medical compositions via the internet. The term “individual” may refer to a human or non-human animal of any age, and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

While CAR-T cell based treatment modalities have displayed unprecedented patient responses and offer a significant curative potential for certain hematological malignancies, success in other cancers and especially in solid tumors remains elusive, in part due to the unique features of the tumor microenvironment characterized by hypoxia, acidic pH, nutrition depletion and/or immunosuppression. Acidity is a prominent feature of the tumor microenvironment primarily due to acidic metabolites, e.g. lactic acid caused by active glycolysis. Acidity mediates immunosuppression, tumor progression and poor prognosis. Specifically, tissue acidosis leads to suppression of immune cell-mediated responses, such as a decrease in natural killer (NK)- and T-cell cytotoxicity, cytokine production and tumor surveillance.

The CAMP pathway mediates major immunosuppressive effects on effector cells and has been implicated in immune cell responses to acidity and hypoxia through certain G-protein coupled receptors (GPRs). For instance, proton receptors are transmembrane GPRs that act as sensors of extracellular acidity, resulting in adenylyl cyclase activity and subsequent CAMP accumulation. This in turn leads to activation of transcription factors in the CAMP pathway that promote anti-inflammatory responses and lead to suppression of immune cell function. As described herein, in some embodiments, key elements in the immune cell response to acidity were genetically engineered. In some embodiments, the cAMP signaling pathway in immune effector cells was targeted for engineered mutation. In some embodiments, mutation of the CAMP signaling pathway can render cellular therapies insensitive to the immunosuppressive effects of acidity and hence increase the cells survival, proliferation, and/or immune function in an acidic tumor microenvironment.

In some embodiments, technologies described herein comprise the use of gene-editing technologies (e.g., CRISPR-Cas technology), wherein the technologies are utilized to mutate (e.g., knock-out) genetic elements disclosed herein. In some embodiments, the prominent and key transcription factor cAMP response element modulator (CREM) is mutated (e.g., knocked-out). As shown herein, in some embodiments, knocking-out CREM from NK cells led to improvement in NK cell persistence and proliferation, as well as antitumor activity against cell lines of cancers characterized by active glycolysis and prominent acidosis of their microenvironment. In some embodiments, a targeted genetic engineering strategy is utilized to target CREM and/or other genes in the CAMP signaling pathway, and/or genes encoding proton receptors as disclosed herein. In some embodiments, genetic engineering strategies are utilized in different forms of cellular therapies, including CAR-T cells, T cells, CAR-NK cells, NK cells, T-cell receptor (TCR)-T cells, TCR-NK cells, and/or tumor-infiltrating lymphocytes (TILs). In some embodiments, such genetic engineering potentiates cell therapies against various types of cancers, including against solid tumors.

In some embodiments, disclosed herein are compositions and methods comprising genetically engineered immune effector cells and/or methods of making the same. In some embodiments, genetic engineering comprises knocking-out of genes encoding proteins that are involved in acidity sensing and downstream signaling by the immunosuppressive cAMP pathway, including but not limited to the following: CREM, ICER, CREB1, GPR4, GPR31, GPR68, GPR81, GPR151, and/or GPR132. In some embodiments, the genetic engineering is performed through the use of endonuclease mediated cleavage (e.g., CRISPR-Cas technology). In some embodiments, compositions disclosed herein are utilized in methods of cancer treatment.

In certain embodiments, the genetic knock-out of genes implicated in responses to acidity and/or their associated downstream signaling pathway components improves an engineered immune cells effectiveness against various tumors. For example, CAR-T cells, that are FDA-approved for the treatment of leukemia, lymphoma, and myeloma, could be genetically engineered to comprise a mutation in one or more of these genes: CREM, ICER, CREB1, GPR4, GPR31, GPR68, GPR81, GPR151, and/or GPR132, in an attempt to increase their effectiveness in the acidic TME of various cancers, which could potentially lead to expansion of this therapy to resistant tumors such as solid tumors. Moreover, this genetic engineering strategy could be used in various other forms of cellular therapies such as CAR-NK cells, TCR-T cells, TILs, to potentiate their activity against various cancer types.

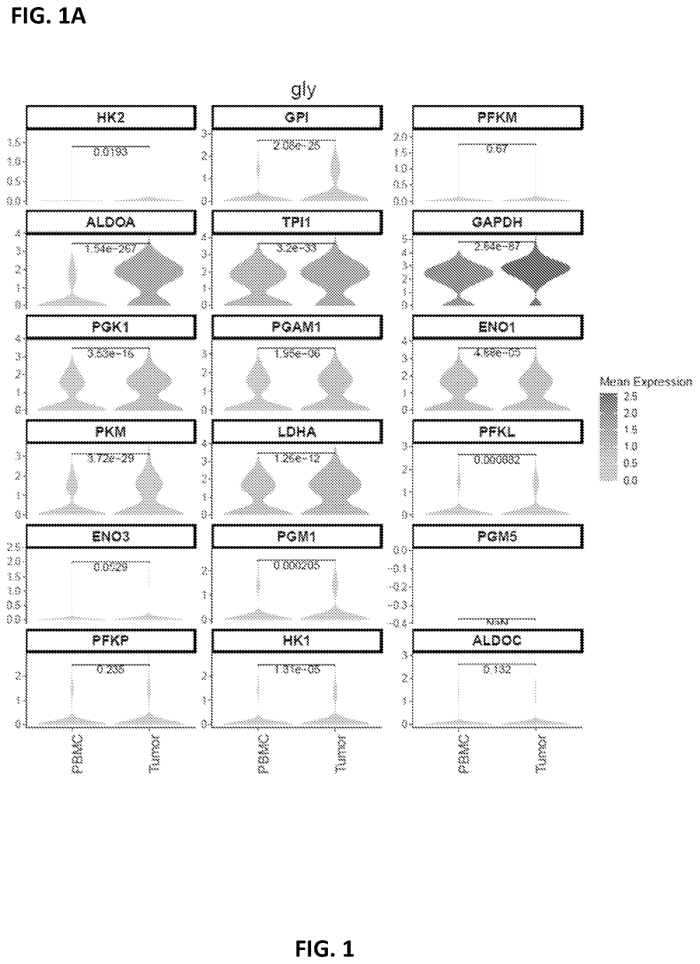

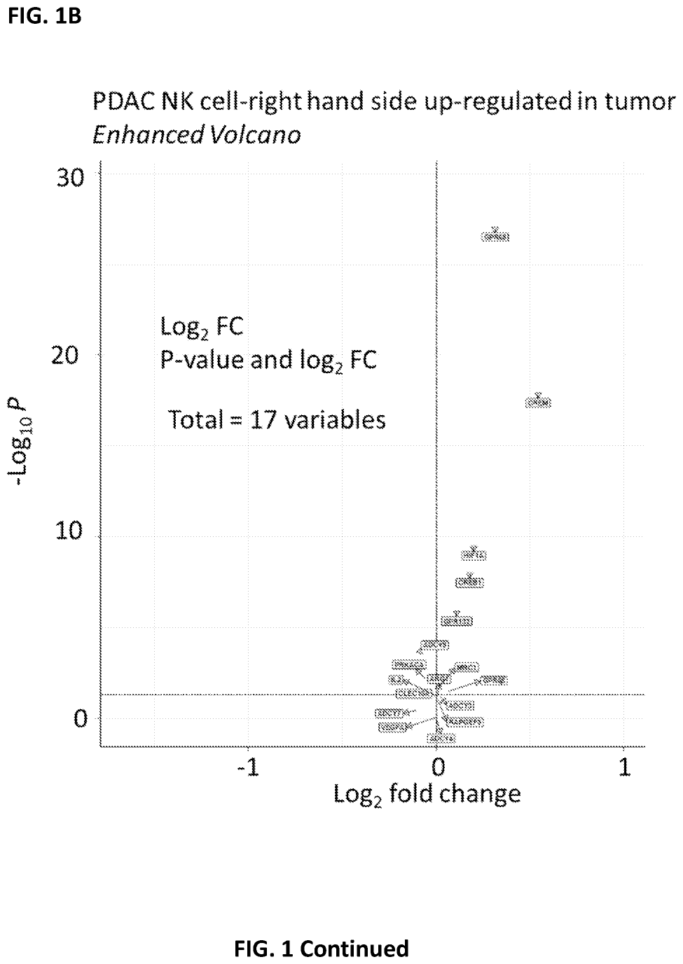

As described herein, single cell RNA sequencing data was analyzed and the results showed high expression of proton-sensing GPRs and the transcription factor CREM in tumor infiltrating immune cells compared to their peripheral blood counterparts (FIGS. 1A and 1B). CREM is a transcriptional repressor in the cAMP pathway that is upregulated in response to acidity signals through proton-sensing GPRs. In some embodiments, the CAMP signaling pathway is modulated through mutation of the CREM transcription factor. In some embodiments, the effect of CREM mutations is analyzed in cord blood-derived NK and CAR-NK cells. In some embodiments, gene engineering technologies, such as CRISPR-Cas9 editing systems were employed to genetically engineer target genes. In some embodiments, one or more guide RNA molecules were designed to the target genes of interest described herein. In some embodiments, one or more guide RNA molecules were utilized to generate mutations in the target genes of interest described herein. In some embodiments, a target gene of interest is specifically CREM. In some embodiments, mutation of CREM (e.g., knock-out of CREM) provides immune effector cells (e.g., NK cells) with a significant proliferative advantage over CREM wild type (WT) cells (e.g., WT NK cells). In some embodiments, mutation of CREM (e.g., knock-out of CREM) provides immune effector cells (e.g., NK cells) with an immunophenotype characterized by activation and cytotoxicity. In some embodiments, mutation of CREM (e.g., CREM KO) provides immune effector cells (e.g., NK cells) with enhanced metabolic fitness. In some embodiments, enhanced metabolic fitness is shown by an increase in oxidative phosphorylation and/or glycolytic activity. In some embodiments, CREM KO effector cells (e.g., NK cells) with or without additional engineering (e.g., with or without chimeric antigen receptors (CARs), etc.) have increased cytotoxicity over CREM WT effector cells (e.g., NK cells) with or without additional engineering (e.g., with or without CARs, etc.). In some embodiments, improvements in cytotoxicity associated with CREM mutation are particularly acute when examined under acidic pH conditions that would normally suppress effector cell cytotoxicity. In some embodiments, mutations in CREM (e.g., knock-out of CREM) improves long-term cytotoxicity, in either conventional 2D cultures and/or 3D cultures where tumor cells grow in spheroids to mimic the features of solid tumors. In some embodiments, mutations in CREM (e.g., knock-out of CREM) improves long-term cytotoxicity in in-vivo solid tumor microenvironments. In some embodiments, a tumor microenvironment is characterized by increased levels of lactic acid relative to a non-tumor microenvironment.

II. Gene Editing of Cells Having Reduced or Inhibited Level of GPR4, GPR31, GPR68, GPR81, GPR132, GPR151, CREM, ICER, and/or CREB1 Expression

Prior to expansion and genetic modification of the cells of the disclosure, a source of cells can be obtained from a subject through a variety of non-limiting methods. Immune cells of any kind, such as NK cells, can be obtained from a number of non-limiting sources, including from peripheral blood mononuclear cells, bone marrow, lymph node tissue, cord blood, thymus tissue, tissue from a site of infection, ascites, pleural effusion, spleen tissue, tumors, or commercially available. Any number of immune cell lines available and known to those skilled in the art, may be used.

In particular embodiments, immune effector cells of any kind are gene edited to modify expression of endogenous GPR4, GPR31, GPR68, GPR81, GPR132, GPR151, CREM, ICER, and/or CREB1 in the cell. In specific cases, the cells are modified to have reduced levels of expression of GPR4, GPR31, GPR68, GPR81, GPR132, GPR151, CREM, ICER, and/or CREB1, including complete inhibition of detectable expression of certain isoforms of GPR4, GPR31, GPR68, GPR81, GPR132, GPR151, CREM, ICER, and/or CREB1 (e.g., that may be referred to as knocked out). Such cells may or may not be expanded prior to production and/or prior to use.

In particular cases, the GPR4, GPR31, GPR68, GPR81, GPR132, GPR151, CREM, ICER, and/or CREB1 gene is disrupted in expression where the expression is reduced in part or in full. In specific cases, the GPR4, GPR31, GPR68, GPR81, GPR132, GPR151, CREM, ICER, and/or CREB1 gene is knocked down or knocked out using processes of the disclosure. In certain embodiments, the GPR4, GPR31, GPR68, GPR81, GPR132, GPR151, CREM, ICER, and/or CREB1 are disrupted (e.g., mutated) in such a way that one or more RNA isoforms encoded by the mutated aforementioned one or more genes are upregulated relative to a non-mutated copy of the same gene.

A skilled artisan is aware how to engineer any cell, including any immune cell, to have reduced or fully inhibited expression of GPR4, GPR31, GPR68, GPR81, GPR132, GPR151, CREM, ICER, and/or CREB1 gene. Particular embodiments utilize means that encompass targeting of the polynucleotide sequence of the specific gene desired to be reduced or fully inhibited in expression.

In some embodiments, the GPR4, GPR31, GPR68, GPR81, GPR132, GPR151, CREM, ICER, and/or CREB1 gene is disrupted in a heterozygous manner. In some embodiments, the GPR4, GPR31, GPR68, GPR81, GPR132, GPR151, CREM, ICER, and/or CREB1 is disrupted in a homozygous manner. In some embodiments, a population of immune effector cells that have a disruption the GPR4, GPR31, GPR68, GPR81, GPR132, GPR151, CREM, ICER, and/or CREB1 gene comprise immune cells that are homozygous for wild type genes, are heterozygous for a wildtype gene and a disrupted gene, and/or homozygous for disrupted genes. In some embodiments, the GPR4, GPR31, GPR68, GPR81, GPR132, GPR151, CREM, ICER, and/or CREB1 gene is disrupted in a majority of alleles in a population of immune effector cells. In some embodiments, the GPR4, GPR31, GPR68, GPR81, GPR132, GPR151, CREM, ICER, and/or CREB1 gene is disrupted in greater than or equal to, about or exactly 50%, 55%, 60%, 65%, 70%, 75%, 80%, 85%, 90%, 95%, 99%, or 100%, or any range derivable therein, of alleles for the gene in the population.

An example of a G protein-coupled receptor 4 ((GPR4), also known as GPR6C.1) gene sequence is in the National Center for Biotechnology Information GENBANK® Database under gene ID 2828 (GPR4) at NC_000019.10 Reference GRCh38.p14 Primary Assembly (range, 45589764 to 45602212 complement) for the genomic sequence, NM_005282.3 for an mRNA sequence, and NP_005273.1 for a protein sequence, each of which are incorporated by reference herein in their entirety. In some embodiments, a GPR4 is mutated as a result of exposure of the cell to a polynucleotide comprising a sequence provided in Table 2. In some embodiments, a GPR4 is mutated as a result of exposure of the cell to a polynucleotide comprising a sequence at least 85%, 86%, 87%, 88%, 89%, 90%, 91%, 92%, 93%, 94%, 95%, 96%, 97%, 98%, 99%, or 100% identical to any one or more of SEQ ID NOs: 3-25.

An example of a G protein-coupled receptor 31 ((GPR31), also known as HETER; HETERI; and/or 12-HETER) gene sequence is in the National Center for Biotechnology Information GENBANK® Database under gene ID 2853 (GPR31) at NC_000006.12 Reference GRCh38.p14 Primary Assembly (range, 167155247 to 167157980 complement) for the genomic sequence, NM_005299.3 for an mRNA sequence, and NP_005290.2 for a protein sequence, each of which are incorporated by reference herein in their entirety. In some embodiments, a GPR31 is mutated as a result of exposure of the cell to a polynucleotide comprising a sequence provided in Table 2. In some embodiments, a GPR31 is mutated as a result of exposure of the cell to a polynucleotide comprising a sequence at least 85%, 86%, 87%, 88%, 89%, 90%, 91%, 92%, 93%, 94%, 95%, 96%, 97%, 98%, 99%, or 100% identical to any one or more of SEQ ID NOs: 26-42.

An example of a G protein-coupled receptor 68 ((GPR68) also known as OGR1; A12A6; and/or GPR12A) gene sequence is in the National Center for Biotechnology Information GENBANK® Database under gene ID 8111 (GPR68) at NC_000014.9 Reference GRCh38.p14 Primary Assembly (range 91232532 to 91270790, complement) for the genomic sequence, NM_001177676.2 for an mRNA sequence encoding transcript variant 1, NM 003485.3 for an mRNA sequence encoding transcript variant 2, NM_001348437.1 for an mRNA sequence encoding transcript variant 3, and NP_001171147.1, NP_001335366.1, and NP 003476.3 for a protein sequence, each of which are incorporated by reference herein in their entirety. In some embodiments, a GPR68 is mutated as a result of exposure of the cell to a polynucleotide comprising a sequence provided in Table 2. In some embodiments, a GPR68 is mutated as a result of exposure of the cell to a polynucleotide comprising a sequence at least 85%, 86%, 87%, 88%, 89%, 90%, 91%, 92%, 93%, 94%, 95%, 96%, 97%, 98%, 99%, or 100% identical to any one or more of SEQ ID NOs: 43-74.

An example of a G protein-coupled receptor 81 ((GPR81) also known as hydroxycarboxylic acid receptor 1 (HCAR1); HCA1; LACR1; FKSG80; GPR104; TAGPCR; and/or TA-GPCR) gene sequence is in the National Center for Biotechnology Information GENBANK® Database under gene ID 27198 (HCAR1) at NC_000012.12 Reference GRCh38.p14 Primary Assembly (range 122726076 to 122730844, complement) for the genomic sequence, NM_032554.4 for an mRNA sequence, and NP_115943.1 for a protein sequence, each of which are incorporated by reference herein in their entirety. In some embodiments, a GPR81 is mutated as a result of exposure of the cell to a polynucleotide comprising a sequence provided in Table 2. In some embodiments, a GPR81 is mutated as a result of exposure of the cell to a polynucleotide comprising a sequence at least 85%, 86%, 87%, 88%, 89%, 90%, 91%, 92%, 93%, 94%, 95%, 96%, 97%, 98%, 99%, or 100% identical to any one or more of SEQ ID NOs: 75-94.

An example of a G protein-coupled receptor 132 ((GPR132) also known as G2A) gene sequence is in the National Center for Biotechnology Information GENBANK® Database under gene ID 29933 (GPR132) at NC_000014.9 Reference GRCh38.p14 Primary Assembly (range 105049395 to 105065430, complement) for the genomic sequence, NM_001278694.2 and NM_013345.4 for mRNA sequences encoding protein isoform 1, NM_001278695.2 for an mRNA sequence encoding protein isoform 2, NM_001278696.2 for an mRNA sequence encoding protein isoform 3, NP_001265623.1 and NP_037477.1 for the sequence of protein isoform 1, NP_001265624.1 for the sequence of protein isoform 2, and NP_001265625.1 for the sequence of protein isoform 3, each of which are incorporated by reference herein in their entirety. In some embodiments, a GPR132 is mutated as a result of exposure of the cell to a polynucleotide comprising a sequence provided in Table 2. In some embodiments, a GPR132 is mutated as a result of exposure of the cell to a polynucleotide comprising a sequence at least 85%, 86%, 87%, 88%, 89%, 90%, 91%, 92%, 93%, 94%, 95%, 96%, 97%, 98%, 99%, or 100% identical to any one or more of SEQ ID NOs: 95-119.

An example of a G protein-coupled receptor 151 ((GPR151) also known as GPCR; PGR7; GALR4; GALRL; and/or GPCR-2037) gene sequence is in the National Center for Biotechnology Information GENBANK® Database under gene ID 134391 (GPR151) at NC 000005.10 Reference GRCh38.p14 Primary Assembly (range 146513144 to 146516190 complement) for the genomic sequence, NM_194251.3 for an mRNA sequence, and NP_919227.2 for a protein sequence, each of which are incorporated by reference herein in their entirety. In some embodiments, a GPR151 is mutated as a result of exposure of the cell to a polynucleotide comprising a sequence provided in Table 2. In some embodiments, a GPR151 is mutated as a result of exposure of the cell to a polynucleotide comprising a sequence at least 85%, 86%, 87%, 88%, 89%, 90%, 91%, 92%, 93%, 94%, 95%, 96%, 97%, 98%, 99%, or 100% identical to any one or more of SEQ ID NOs: 120-139.

An example of a cAMP responsive element modulator ((CREM) also known as ICER; CREM-2; and/or hCREM-2) gene sequence is in the National Center for Biotechnology Information GENBANK® Database under gene ID 1390 (CREM) at NC_000010.11 Reference GRCh38.p14 Primary Assembly (range 35126846 to 35212958) for the genomic sequence, or NG_029065.1 RefSeqGene (range 5129 to 91081), each of which are incorporated by reference herein in their entirety. The CREM gene encodes numerous RNA and protein isoforms, some of which are inducible cAMP Early Repressor (ICER) RNA isoforms encoding ICER proteins. Examples of CREM mRNA isoform sequences include but are not limited to: NM 001267562.2, NM_001267563.2, NM 001267564.2, NM 001267565.2, NM_001267566.2, NM 001267567.2, NM 001267568.2, NM 001267569.2, NM 001267570.2, NM 001352445.1, NM_001352446.1, NM 001352465.2, NM 001352466.2, NM_001352467.2, NM_001394595.1, NM 001394598.1, NM 001394600.1, NM 001394602.1, NM_001394603.1, NM 001394605.1, NM 001394608.1, NM 001394610.1, NM_001394613.1, NM 001394614.1, NM 001394615.1, NM 001394616.1, NM_001394617.1, NM 001394618.1, NM_001394619.1, NM 001394620.1, NM 001394621.1, NM 001394622.1, NM_001394623.1, NM_001394625.1, NM_001394626.1, NM 001394627.1, NM_001394628.1, NM_001394629.1, NM_001394630.1, NM_001394631.1, NM_001881.4, NM 181571.3, NM_182717.2, NM_182718.2, NM_182719.2, NM_182720.2, NM_182721.2, NM_182723.2, NM_182724.2, NM_182769.3, NM_182770.3, NM_182771.2, NM_182772.2, NM 183011.2, NM_183012.2, NM_183013.3, and NM_183060.3, each of which are incorporated by reference herein in their entirety. Examples of CREM protein isoform sequences include but are not limited to: NP_001254491.1, NP_001254492.1, NP_001254493.1, NP_001254494.1, NP_001254495.1, NP_001254496.1, NP_001254497.1, NP_001254498.1, NP_001254499.1, NP_001339374.1, NP_001339375.1, NP_001339394.1, NP_001339395.1, NP_001339396.1, NP_001381524.1, NP_001381527.1, NP_001381529.1, NP_001381531.1, NP_001381532.1, NP_001381534.1, NP_001381537.1, NP_001381539.1, NP 001381542.1, NP_001381543.1, NP_001381544.1, NP_001381545.1, NP_001381546.1, NP_001381547.1, NP_001381548.1, NP_001381549.1, NP_001381550.1, NP_001381551.1, NP 001381552.1, NP_001381554.1, NP_001381555.1, NP_001381556.1, NP_001381557.1, NP_001381558.1, NP_001381559.1, NP_001381560.1, NP_001872.3, NP_853549.1, NP_874386.1, NP_874387.1, NP_874388.1, NP_874389.1, NP_874390.1, NP_874392.1, NP 874393.1, NP_877570.1, NP_877571.1, NP_877572.1, NP_877573.1, NP_898829.1, NP_898830.1, NP_898831.1, and NP_898883.1, each of which are incorporated by reference herein in their entirety. The CREM RNA isoforms encoding the ICER protein include NM_182717.2, NM_182718.2, NM_182719.2, NM_182720.2, NM_182721.2, NM_182723.2, NM 182724.2, while the ICER protein variant sequences are represented by NP_874386.1 (isoform ICER1), NP_874387.1 (isoform ICER11 gamma), NP_874388.1 (isoform ICER11), NP_874389.1 (isoform ICER1 gamma), NP_874390.1 (isoform 8 aka h), NP_874392.1 (isoform 10 aka j), and NP_874393.1 (isoform 11 aka k). In some embodiments, a CREM is mutated as a result of exposure of the cell to a polynucleotide comprising a sequence provided in Table 2. In some embodiments, a CREM is mutated as a result of exposure of the cell to a polynucleotide comprising a sequence at least 85%, 86%, 87%, 88%, 89%, 90%, 91%, 92%, 93%, 94%, 95%, 96%, 97%, 98%, 99%, or 100% identical to any one or more of SEQ ID NOs: 140-161, and 181-184. In some embodiments, a CREM is mutated as a result of exposure of the cell to a polynucleotide comprising a sequence at least 85%, 86%, 87%, 88%, 89%, 90%, 91%, 92%, 93%, 94%, 95%, 96%, 97%, 98%, 99%, or 100% identical to SEQ ID NOs: 140 and/or 142.

An example of a cAMP responsive element binding protein 1 ((CREB1) also known as CREB; and/or CREB-1) gene sequence is in the National Center for Biotechnology Information GENBANK® Database under gene ID 1385 (CREB1) at NC_000002.12 Reference GRCh38.p14 Primary Assembly (range 207529962 to 207605988) for the genomic sequence, while mRNA sequences are represented by but not limited to: NM_001320793.2, NM_001371426.1, NM_001371427.1, NM_001371428.1, NM_004379.5, NM_134442.5, and protein sequences are represented by but not limited to: NP_001307722.1, NP_001358355.1, NP_001358356.1, NP_001358357.1, NP_004370.1, and NP_604391.1, each of which are incorporated by reference herein in their entirety. In some embodiments, a CREB1 is mutated as a result of exposure of the cell to a polynucleotide comprising a sequence provided in Table 2. In some embodiments, a CREB1 is mutated as a result of exposure of the cell to a polynucleotide comprising a sequence at least 85%, 86%, 87%, 88%, 89%, 90%, 91%, 92%, 93%, 94%, 95%, 96%, 97%, 98%, 99%, or 100% identical to any one or more of SEQ ID NOs: 162-180. In some embodiments, 5′-3′ DNA sequences of exemplary guide RNAs for knocking-out of the GPR4, GPR31, GPR68, GPR81, GPR132, GPR151, CREM, ICER, and/or CREB1 gene are found in Table 2. In some embodiments, these guide RNA sequences are suitable for use with CRISPR/Cas9 technology which utilizes guide RNAs (complementary to short target DNA sequences on the targeted gene) in order to perform double-stranded DNA cleavage. Guide RNAs could be positively stranded or negatively stranded but since the cleavage made using CRISPR/Cas9 technology affects both strands of the target DNA, shown here is the target sequence on the positive DNA strand of the sequence.

In some embodiments, following mutation with an endonuclease, oligonucleotide amplification techniques (e.g., PCR) can be utilized to determine mutation efficiency and/or mutation type. In some embodiments, for a PCR reaction, primers that encompass (e.g., flank) an edited region are utilized to amplify a target sequence. In some embodiments, a suitable primer for amplification of a target locus may be but is not limited to SEQ ID NOs: 1 and 2.

TABLE 1
Exemplary primer sequences

| Target | Forward Primer | Reverse Primer |
|---|---|---|
| sequence | (5′ to 3′) | (5′ to 3′) |
| CDS of CREM | TGAATGAACTGTCC | CCTGAGTTGCTTCA |
| | TCTGATGTG | ATATAACTAGAGA |
| | (SEQ ID NO: 1) | (SEQ ID NO: 2) |

Embodiments of the disclosure include methods of knocking out or down expression of endogenous GPR4, GPR31, GPR68, GPR81, GPR132, GPR151, CREM, ICER, and/or CREB1 in a cell, comprising contacting the cell at least with Cas9, or a functionally equivalent alternative, and an appropriate guide RNA that targets GPR4, GPR31, GPR68,

GPR81, GPR132, GPR151, CREM, ICER, and/or CREB1. The Cas9 and/or guide RNA may be provided to the cell through expression from one or more expression vectors coding therefor. The vector may be viral (retroviral, lentiviral, adenoviral, adeno-associated viral) or non-viral (naked plasmid DNA or chemically-modified mRNA).

In specific cases, other gene(s) than GPR4, GPR31, GPR68, GPR81, GPR132, GPR151, CREM, ICER, and/or CREB1 are knocked down or knocked out, and this may or may not occur in the same step as the GPR4, GPR31, GPR68, GPR81, GPR132, GPR151, CREM, ICER, and/or CREB1 knock out or knock down. The reduction or full inhibition of expression may or may not utilize the same mechanism of gene editing as that for GPR4, GPR31, GPR68, GPR81, GPR132, GPR151, CREM, ICER, and/or CREB1, and the reduction or full inhibition of expression of the other gene(s) may occur before, during, or after the gene editing for GPR4, GPR31, GPR68, GPR81, GPR132, GPR151, CREM, ICER, and/or CREB1. The genes that are edited in the cells may be of any kind, but in specific embodiments the genes are genes whose gene products inhibit activity and/or proliferation of the GPR4, GPR31, GPR68, GPR81, GPR132, GPR151, CREM, ICER, and/or CREB1 KO cells. In specific cases the genes that are edited in addition to GPR4, GPR31, GPR68, GPR81, GPR132, GPR151, CREM, ICER, and/or CREB 1 allow the cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, CD38, and CD7. In specific embodiments, the TGFBR2 gene is knocked out or knocked down in the cells. In specific embodiments, the CISH gene is knocked out or knocked down in the cells. In specific embodiments, the CD38 gene is knocked out or knocked down in the cells. In specific embodiments, the Glucocorticoid receptor (GR) gene is knocked out or knocked down in the cells.

In some embodiments, any gene editing in the cells is carried out using one or more DNA-binding molecules, such as an endonuclease, for example but not limited to, Cas enzymes (and variants thereof), zinc-finger nucleases, TALENs, and Meganucleases, etc. In some embodiments, any gene editing in the cells is carried out by one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins; in some embodiments, Cpf1 is utilized instead of Cas9. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). In some cases, Cpf1 may be used as an endonuclease instead of Cas9. The CRISPR enzyme can exert direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US20110059502, incorporated herein by reference.

In some embodiments, a gene editing molecule includes a DNA-binding protein such as one or more zinc finger protein (ZFP) or transcription activator-like protein (TAL), fused to an effector protein such as an endonuclease. Examples include at least ZFNs, TALES, and TALENs.

In some embodiments, a gene editing molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type IIS restriction enzyme and one or more zinc finger binding domains, which may or may not be engineered. In some embodiments, the cleavage domain is from the Type IIS restriction endonuclease Fok I. Fok I generally catalyzes double-stranded cleavage of DNA, at 9 nucleotides from its recognition site on one strand and 13 nucleotides from its recognition site on the other. Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 10 2013, 31 (7), 397-405). In some embodiments, commercially available zinc fingers are used or are custom designed. (See, for example, Sigma-Aldrich catalog numbers CSTZFND, CSTZFN, CTil-IKT, and PZD0020).

In some embodiments, a gene editing molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety herein. A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Di-residue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to Gor A, and NO binds to T and noncanonical (atypical) RVDs are also known. In some embodiments, TALEs may be targeted to any gene by design of TAL arrays with specificity to the target DNA sequence. The target sequence generally begins with a thymidine. In some embodiments, the molecule is a DNA binding endonuclease, such as a TALE nuclease (TALEN). In some aspects the TALEN is a fusion protein comprising a DNA binding domain derived from a TALE and a nuclease catalytic domain to cleave a nucleic acid target sequence. In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art In some embodiments, TALE repeats are assembled to specifically target a gene. (Gaj et al., 2013). A library of TALENs targeting 18,740 human protein-coding genes has been constructed (Kim et al., 2013). Custom-designed TALE arrays are commercially available through Cellectis Bioresearch (Paris, France), Transposagen Biopharmaceuticals (Lexington, KY, USA), and Life Technologies (Grand Island, NY, USA). Specifically, TALENs that target CD38 are commercially available (See Gencopoeia, catalog numbers HTN222870-1, HTN222870-2, and HTN222870-3). Exemplary molecules are described, e.g., in U.S. Patent Publication Nos. US 2014/0120622, and 2013/0315884. In some embodiments, TALENs are introduced as trans genes encoded by one or more plasmid vectors. In some aspects, the plasmid vector can contain a selection marker which provides for identification and/or selection of cells which received said vector.

### III. Immune Effector Cells

The disclosure concerns genetically engineering immune effector cells to comprise a partial reduction or full inhibition of expression of GPR4, GPR31, GPR68, GPR81, GPR132, GPR151, CREM, ICER, and/or CREB1. In some embodiments, the partial reduction or full inhibition of expression of GPR4, GPR31, GPR68, GPR81, GPR132, GPR151, CREM, ICER, and/or CREB1 may occur by any mechanism, including at least by CRISPR/Cas9 technology, to make innovative and effective cellular therapies for the treatment of cancer of any kind, including solid tumors.

The present disclosure encompasses immune effector cells of any kind that are modified to have reduced or fully inhibited expression of GPR4, GPR31, GPR68, GPR81, GPR132, GPR151, CREM, ICER, and/or CREB1. In specific embodiments, the present disclosure encompasses immune effector cells of any kind that are modified to have reduced or fully inhibited expression of CREM. In specific embodiments, the reduction or full inhibition of expression of GPR4, GPR31, GPR68, GPR81, GPR132, GPR151, CREM, ICER, and/or CREB1 in the cells is a direct or indirect result of deliberate manipulation of the cells by the hand of man. The manipulation of the immune effector cells to have reduced or fully inhibited expression of GPR4, GPR31, GPR68, GPR81, GPR132, GPR151, CREM, ICER, and/or CREB1 may be by any mechanism, including by homologous or non-homologous recombination. In specific embodiments, the cells are manipulated to have reduced or fully inhibited expression of GPR4, GPR31, GPR68, GPR81, GPR132, GPR151, CREM, ICER, and/or CREB1 as a result of CRISPR technology, for example.

The immune effector cells have reduced or inhibited expression of GPR4, GPR31, GPR68, GPR81, GPR132, GPR151, CREM, ICER, and/or CREB1 particularly by genetic engineering, as opposed to natural cells having one or more mutations that result in reduced expression of endogenous GPR4, GPR31, GPR68, GPR81, GPR132, GPR151, CREM, ICER, and/or CREB1. Thus, in specific embodiments the immune effector cells are genetically engineered to reduce or inhibit expression of the endogenous GPR4, GPR31, GPR68, GPR81, GPR132, GPR151, CREM, ICER, and/or CREB1 in the genome of the immune effector cells. In specific embodiments, the immune effector cells are knocked out for expression of endogenous GPR4, GPR31, GPR68, GPR81, GPR132, GPR151, CREM, ICER, and/or CREB1.

The present disclosure encompasses immune effector cells of any kind, including conventional T cells, gamma-delta T cells, NK cells, NK T cells, invariant NK T cells, regulatory T cells, macrophages, B cells, dendritic cells, tumor-infiltrating lymphocytes, MSCs, or a mixture thereof. The cells may be allogeneic, autologous, or xenogeneic with respect to an individual, including an individual in need of the cells, such as an individual with cancer.

In particular embodiments, the immune effector cells are modified by the hand of man to express or otherwise produce one or more gene products other than the cell also being modified to have reduced or fully inhibited expression of GPR4, GPR31, GPR68, GPR81, GPR132, GPR151, CREM, ICER, and/or CREB1. Such additional modification(s) to the cell are not naturally present in the cell or are of exogenous origin with respect to the cell. The additional modification(s) may be of any kind, such as the immune effector cells expressing a receptor, a cytokine, a suicide gene, or a chemokine, or a combination thereof, as examples.

When the immune effector cells having reduced or fully inhibited expression of GPR4, GPR31, GPR68, GPR81, GPR132, GPR151, CREM, ICER, and/or CREB1 are also modified additionally to produce or express a gene product that is not naturally present in the cell or is of exogenous origin, the order in which the immune effector cell is modified may be of any kind. For example, immune effector cells having reduced or fully inhibited expression of GPR4, GPR31, GPR68, GPR81, GPR132, GPR151, CREM, ICER, and/or CREB1 may be modified to have one or more additional modifications, wherein in other cases immune effector cells are modified to have reduced or fully inhibited expression of GPR4, GPR31, GPR68, GPR81, GPR132, GPR151, CREM, ICER, and/or CREB1 after they have been modified to produce or express a gene product that is not naturally present in the cell or is of exogenous origin.

In particular embodiments, the immune effector cells lacking full or partial expression of GPR4, GPR31, GPR68, GPR81, GPR132, GPR151, CREM, ICER, and/or CREB1 is the same cell that is modified to express a receptor, such as an antigen receptor. Any immune effector cell encompassed by the present disclosure expresses an antigen receptor that may be of any kind, including a receptor directed towards an antigen that is a cancer antigen that may also be a tumor antigen. In specific embodiments, the receptor is a chimeric antigen receptor or a T-cell receptor, for example. The immune effector cells may be specifically designed to have full or partial inhibition of expression of GPR4, GPR31, GPR68, GPR81, GPR132, GPR151, CREM, ICER, and/or CREB1 and is specifically designed to have an antigen receptor that targets an antigen on cancer cells in the individual. That is, the cells may be tailored to include one or more antigen receptors that target antigens known to be present on cancer cells of the individual.

In particular embodiments, cells of the present disclosure are produced for the purpose of being used as off-the-shelf cells. For example, cells that have full or partial inhibition of expression of GPR4, GPR31, GPR68, GPR81, GPR132, GPR151, CREM, ICER, and/or CREB1 are present in a repository, for example, and they are obtained from the repository and engineered to have a further modification other than have full or partial inhibition of expression of GPR4, GPR31, GPR68, GPR81, GPR132, GPR151, CREM, ICER, and/or CREB1. In other cases, cells that have a modification other than having full or partial inhibition of expression of GPR4, GPR31, GPR68, GPR81, GPR132, GPR151, CREM, ICER, and/or CREB1 are obtained from a repository and are engineered to have full or partial inhibition of expression of GPR4, GPR31, GPR68, GPR81, GPR132, GPR151, CREM, ICER, and/or CREB1. Following such modifications to the cells after obtaining them from a repository, the cells may be stored, or an effective amount of the cells are provided to an individual in need thereof. Further engineering of GPR4, GPR31, GPR68, GPR81, GPR132, GPR151, CREM, ICER, and/or CREB1 KO or knock-down cells may be to engineer them to express an engineered receptor, such as an engineered antigen receptor that targets a tumor antigen suitable for treatment of an individual with a specific cancer expressing antigen.

In particular embodiments, the immune effector cells have full or partial inhibition of expression of GPR4, GPR31, GPR68, GPR81, GPR132, GPR151, CREM, ICER, and/or CREB1 and also express one or more engineered antigen-targeting receptors and/or express at least one transfected (as opposed to endogenous to the cell) cytokine and/or express at least one suicide gene. In some cases of cells having full or partial inhibition of expression of GPR4, GPR31, GPR68, GPR81, GPR132, GPR151, CREM, ICER, and/or CREB1, different vectors encode the antigen-targeting receptor(s) vs. encode the suicide gene(s) and/or transfected cytokine(s). The immune cells, including NK cells, may be derived from cord blood, peripheral blood, induced pluripotent stem cells (iPSCs), hematopoietic stem cells (HSCs), bone marrow, or a mixture thereof. The NK cells may be derived from a cell line such as, but not limited to, NK-92 cells, for example. The NK cell may be a cord blood mononuclear cell, such as a CD56+NK cell.

The present disclosure describes and/or shows successful knock-out (KO) of the GPR4, GPR31, GPR68, GPR81, GPR132, GPR151, CREM, ICER, and/or CREB1 gene using CRISPR/Cas9 from natural killer (NK) cells derived from cord blood stored in cord blood banks. The present disclosure shows how cAMP signaling pathway disrupted NK cells have enhanced antitumor activity over WT cAMP signaling pathway NK cells in acidic conditions or in vivo-like conditions that were shown to be acidic in the literature. This enhanced antitumor activity was shown against solid tumor cell lines that are known to have active glycolysis and a prominent acidic tumor microenvironment. In certain embodiments, a tumor microenvironment is characterized by increased levels of lactic acid relative to non-tumor microenvironments.

In some cases, the immune effector cells having full or partial inhibition of expression of GPR4, GPR31, GPR68, GPR81, GPR132, GPR151, CREM, ICER, and/or CREB1 have been expanded in the presence of an effective amount of universal antigen presenting cells (UAPCs) or fragments thereof, including in any suitable ratio. The cells may be cultured with the UAPCs at a ratio of 10:1 to 1:10; 9:1 to 1:9; 8:1 to 1:8; 7:1 to 1:7; 6:1 to 1:6; 5:1 to 1:5; 4:1 to 1:4; 3:1 to 1:3; 2:1 to 1:2; or 1:1, including at a ratio of 1:2, for example. In some cases, the NK cells were expanded in the presence of IL-2, such as at a concentration of 10-500, 10-400, 10-300, 10-200, 10-100, 10-50, 100-500, 100-400, 100-300, 100-200, 200-500, 200-400, 200-300, 300-500, 300-400, or 400-500 U/mL.

Following genetic modification with any vector(s), the immune effector cells having partial or full reduction of expression of GPR4, GPR31, GPR68, GPR81, GPR132, GPR151, CREM, ICER, and/or CREB1 may be immediately delivered to an individual or may be stored (or some of the cells are delivered to an individual and the rest of the cells are stored). In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid is expanded ex vivo. The clone selected for expansion demonstrates reduced or absence of expression of GPR4, GPR31, GPR68, GPR81, GPR132, GPR151, CREM, ICER, and/or CREB1. The recombinant immune cells may be expanded by stimulation with IL-2, or other cytokines that bind the common gamma-chain (e.g., IL-7, IL-12, IL-15, IL-21, and others). The recombinant immune cells may be expanded by stimulation with artificial antigen presenting cells. In a further aspect, the genetically modified cells may be cryopreserved.

In some embodiments, cells are treated with one or more deactivating agents (e.g., a kinase inhibitor, e.g., Dasatinib, Nilotinib, Rapamycin, etc.) precryopreservation. In some embodiments, technologies described herein comprise deactivating an NK cell, comprising treating an NK cell with an effective amount of one or more deactivating agents under conditions to produce a deactivated NK cell. In some embodiments, a deactivating agent is a kinase inhibitor. In some embodiments, a deactivating agent is a mechanistic target of rapamycin (mTOR) inhibitor. In some embodiments, the mTOR inhibitor is rapamycin, everolimus, and/or temsirolimus. In some embodiments, the mTOR inhibitor is rapamycin. In some embodiments, the deactivating agent is a tyrosine kinase (TK) inhibitor. In some embodiments, the TK inhibitor is Lorlatinib, Brigatinib, Ceritinib, Alectinib, Crizotinib, Bosutinib, Ponatinib, Nilotinib, Dasatinib, Imatinib, Zanubrutinib, Acalabrutinib, Ibrutinib, Capmatinib, Pexidartinib, Dacomitinib, Osimertinib, Erlotinib, Gefitinib, Lapatinib, Afatinib, Pemigatinib, Erdafitinib, Nintedanib, Gilteritinib, Midostaurin, Tucatinib, Neratinib, Baricitinib, Ruxolitinib, Fedratinib, Tofacitinib, Ripretinib, Selumetinib, Binimetinib, Cobimetinib, Trametinib, Upadacitinib, Avapritinib, Selpercatinib, Cabozantinib, Fostamatinib, Larotrectinib, Entrectinib, Axitinib, Regorafenib, Pazopanib, Sorafenib, Lenvatinib, Vandetanib, and/or Sunitinib. In some embodiments, the TK inhibitor is a BCR-Abl inhibitor. In some embodiments, the TK inhibitor is Bosutinib, Ponatinib, Nilotinib, Dasatinib, and/or Imatinib. In some embodiments, the TK inhibitor is Dasatinib and/or Nilotinib. In some embodiments, the TK inhibitor is Dasatinib.

In some embodiments, treatment with a deactivating agent is at any point during culturing of the NK cell. In some embodiments, the treatment is for about 24 to about 96 hours, about 36 to about 84 hours, or about 48 to about 72 hours. In some embodiments, the treatment is for about 24 hours, about 48 hours, or about 72 hours. In some embodiments, the NK cell is treated with the deactivating agent at a concentration of about 1 to about 1000 nM. In some embodiments, the NK cell is treated with the deactivating agent at a concentration of about 5 to about 500 nM. In some embodiments, the NK cell is treated with the deactivating agent at a concentration of about 20 to about 200 nM. In some embodiments, the NK cell is treated with the deactivating agent at a concentration of about 30 to about 100 nM. In some embodiments, the deactivated NK cell has an increased expression of one or more of C-kit, CCR-5, CD62L and/or CXCR4, and/or decreased expression of one or more of NKG2D, DNAM, OX-40, TRAIL, HLA-DR, CD2, CD25, ICOS, and/or CD95 relative to an activated NK cell.

In some embodiments, technologies described herein comprise methods of maintaining the viability of a population of cells over at least 50% percent following cryopreservation of the population, comprising the step of subjecting the population to an effective amount of one or more deactivating agents (e.g., a tyrosine kinase inhibitor) to deactivate the cells prior to cryopreservation, cryopreserving the cells, and thawing the population, wherein upon thawing the viability of the population is over at least 50%. In some cases, upon thawing of the cells the viability of the population of cells is over at least 55, 60, 65, 70, 75, 80, 85, 90, 91, 92, 93, 94, 95, 96, 97, 98, or 99% following cryopreservation of the population.

Embodiments of the disclosure encompass immune effector cells having full or partial inhibition of expression of GPR4, GPR31, GPR68, GPR81, GPR132, GPR151, CREM, ICER, and/or CREB1 and one or more engineered receptors, including one or more antigen receptors. The one or more engineered antigen receptors are generated by the hand of man, for example using recombinant techniques, and are not natural to the immune effector cell. Although the engineered receptor(s) may be of any kind, in specific embodiments the receptor is a chimeric antigen receptor, T-cell receptor, homing receptors, CRISPR/Cas9-mediated gene mutations, decoy receptors, cytokine receptors, chimeric cytokine receptors, and so forth.

Embodiments of the disclosure encompass cells having full or partial inhibition of expression of GPR4, GPR31, GPR68, GPR81, GPR132, GPR151, CREM, ICER, and/or CREB1 and one or more suicide genes. The immune effector cell may have full or partial inhibition of expression of GPR4, GPR31, GPR68, GPR81, GPR132, GPR151, CREM, ICER, and/or CREB1 and may comprise a recombinant nucleic acid that encodes a suicide gene of any kind. Examples of suicide genes include engineered nonsecretable (including membrane bound) tumor necrosis factor (TNF)-alpha mutant polypeptides (see PCT/US2019/062009, which is incorporated by reference herein in its entirety), and they may be affected by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidylate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP), as examples.

The cells may be obtained from an individual directly or may be obtained from a depository or other storage facility. The cells as therapy may be autologous or allogeneic with respect to the individual to which the cells are provided as therapy.

The cells may be from an individual in need of therapy for a medical condition, and following their manipulation to have reduced or inhibited GPR4, GPR31, GPR68, GPR81,

GPR132, GPR151, CREM, ICER, and/or CREB1 expression, optional suicide gene, optional cytokine(s), and optional receptor(s) (using standard techniques for transduction and expansion for adoptive cell therapy, for example), they may be provided back to the individual from which they were originally sourced. In some cases, the cells are stored for later use for the individual or another individual.

The immune cells may be comprised in a population of cells, and that population may have a majority that have reduced or inhibited GPR4, GPR31, GPR68, GPR81, GPR132, GPR151, CREM, ICER, and/or CREB1 expression and/or one or more suicide genes and/or one or more cytokines. A cell population may comprise 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of immune cells that have reduced or inhibited GPR4, GPR31, GPR68, GPR81, GPR132, GPR151, CREM, ICER, and/or CREB1 expression and/or one or more suicide genes and/or one or more cytokines and/or one or more engineered receptor; each of these gene products may or may not be produced as separate polypeptides.

The immune cells may be produced to have reduced or inhibited GPR4, GPR31, GPR68, GPR81, GPR132, GPR151, CREM, ICER, and/or CREB1 expression and/or one or more suicide genes and/or one or more cytokines for the intent of being modular with respect to a specific purpose. For example, cells may be generated, including for commercial distribution, having reduced or inhibited GPR4, GPR31, GPR68, GPR81, GPR132, GPR151, CREM, ICER, and/or CREB1 expression and/or one or more suicide genes and/or one or more cytokines (or distributed with a nucleic acid that encodes a suicide gene for subsequent transduction), and a user may modify them to express one or more other genes of interest (including therapeutic genes) dependent upon their intended purpose(s). For instance, an individual interested in treating cancer cells may obtain or generate suicide gene-expressing cells (or heterologous cytokine-expressing cells) and modify them to have reduced or inhibited GPR4, GPR31, GPR68, GPR81, GPR132, GPR151, CREM, ICER, and/or CREB1 expression, or vice versa.

In particular embodiments, NK cells are utilized, and the genome of the NK cells having reduced or inhibited GPR4, GPR31, GPR68, GPR81, GPR132, GPR151, CREM, ICER, and/or CREB1 expression and/or one or more suicide genes and/or one or more cytokines may be modified. The genome may be modified in any manner, but in specific embodiments the genome is modified by CRISPR gene editing, for example. The genome of the cells may be modified to enhance effectiveness of the cells for any purpose.

Also provided herein, in particular embodiments, are at least methods of making, methods of using, and compositions comprising immune effector cells that have been conditioned (e.g., become accustomed to, adapted to, etc.) to acidic conditions through contact with acidic stimuli. In some embodiments, cells are conditioned ex vivo through contact with acidic stimuli In some embodiments, cells are conditioned incrementally over time. In some embodiments, cells are conditioned incrementally over time by contact to progressively accumulating acidic stimuli. In some embodiments, cells are conditioned over a period of at least or equal to, exactly or about, 1, 2, 3, 4, 5, 6, 7, 8, 9, 10, 11, 12, 13, 14, 15, 16, 17, 18, 19, 20, 21, 21, 23, 24, 25, 26, 27, 28, 29, or 30 days, or greater than 30 days. In some embodiments, cells are conditioned over a period of exactly or about 10 to 18 days. In some embodiments, cells are conditioned over a period of exactly or about 14 days. In some embodiments, cells are conditioned by contacting with acidic stimuli at a concentration greater than or equal to, exactly or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 mM. In some embodiments, cells are conditioned by contacting with acidic stimuli at a concentration greater than or equal to, exactly or about 2-3 mM. In some embodiments, cells are conditioned by contacting with acidic stimuli at a concentration greater than or equal to, exactly or about 2.5 mM. In some embodiments, cells are conditioned with acidic stimuli comprising or consisting essentially of lactic acid. In some embodiments, acidic stimuli is added at least every 1, 2, 3, 4, 5, 6, 7, 8, 9, or 10 days. In some embodiments, acidic stimuli is added about or exactly every 2-3 days. In some embodiments, cells are conditioned to acidic stimuli at a pH of less than or equal to, exactly or about pH 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, or 5.0. In some embodiments, cells are conditioned to acidic stimuli at a pH of less than or equal to, about or exactly pH 6.0 or pH 5.9.

### IV. Methods of Treatment

Embodiments of the disclosure include methods of treatment related to cancer immunotherapy or anti-pathogen immunotherapy, for example, wherein the cancer immunotherapy and anti-pathogen immunotherapy comprise at least compositions comprising immune effector cells having reduced or inhibited levels of expression of GPR4, GPR31, GPR68, GPR81, GPR132, GPR151, CREM, ICER, and/or CREB1. The methods include providing to an individual with cancer and/or a pathogen an effective amount of immune effector cells having reduced or inhibited levels of expression of GPR4, GPR31, GPR68, GPR81, GPR132, GPR151, CREM, ICER, and/or CREB1.

In particular cases, an individual is provided an effective amount of cells having reduced or inhibited levels of expression of GPR4, GPR31, GPR68, GPR81, GPR132, GPR151, CREM, ICER, and/or CREB1. In specific cases, GPR4, GPR31, GPR68, GPR81, GPR132, GPR151, CREM, ICER, and/or CREB1 knock-out using CRISPR/Cas9 is utilized to genetically engineer immune cells used in various cellular therapies to increase their effectiveness against solid tumors, and these cellular therapies are provided to the individual.

As one example, chimeric antigen receptor (CAR)-T cells, such as those that are FDA-approved for the treatment of leukemia and lymphoma, are genetically engineered to delete the GPR4, GPR31, GPR68, GPR81, GPR132, GPR151, CREM, ICER, and/or CREB1 gene, for the purpose of increasing their effectiveness in the acidic TME of solid tumors, which in particular embodiments leads to expansion of this therapy to solid tumors. Moreover, this genetic engineering strategy is used in various other forms of cellular therapies, such as CAR-NK cells, engineered TCR-T cells, tumor-infiltrating lymphocytes (TILs), to potentiate them against various types of solid tumors.

In certain embodiments, cells of the disclosure are provided to an individual for the purpose of improving a medical condition, such as cancer of any kind and/or pathogen infection of any kind. Use of the cells contemplated herein, including pharmaceutical compositions comprising the same, are used for the prevention, treatment, or amelioration of a cancerous disease, such as a tumorous disease, or a pathogen infection. In particular embodiments, the pharmaceutical composition of the present disclosure may be particularly useful in preventing, ameliorating and/or treating cancer, including cancers that may or may not be solid tumors, for example.

In particular embodiments, the present disclosure contemplates, in part, use of cells encompassed herein that can be administered either alone or in any combination with one or more other therapies, and in at least some aspects, together with a pharmaceutically acceptable carrier or excipient. In certain embodiments, any nucleic acid molecules or vectors may be stably integrated into the genome of the cells prior to deliver of the cells to the subject.

Furthermore, the disclosure relates to a method for the prevention, treatment or amelioration of a tumorous disease comprising the step of administering to a subject in the need thereof an effective amount of any cells that have reduced or inhibited level of expression of GPR4, GPR31, GPR68, GPR81, GPR132, GPR151, CREM, ICER, and/or CREB1, as contemplated herein.

In one embodiment, isolated cells obtained by any suitable methods or from cell lines and engineered as encompassed herein may be used as a medicament. The medicament can be used for treating cancer or infections in an individual in need thereof. In one embodiment, the isolated cells according to the disclosure can be used in the manufacture of a medicament for treatment of a cancer or an infection in an individual in need thereof.

In some embodiments, the present disclosure provides methods for treating individuals in need thereof, said methods comprising at least one of the following steps: (a) providing immune effector cells; (b) engineering the immune effector cells to have reduced or inhibited expression of at least GPR4, GPR31, GPR68, GPR81, GPR132, GPR151, CREM, ICER, and/or CREB1; (c) engineering the immune effector cells to express one or more engineered receptors (and step (c) may come at the same time or before step (b); (d) engineering the immune effector cells to express one or more cytokines (and step (d) may come at the same time or before steps (b) or (c); (e) administering the engineered cells to an individual in need thereof, including an individual that has been determined to have cancer or is at risk of having cancer (such as greater than the average person of a population).

In specific embodiments, the engineered cells were engineered specifically for the purpose of producing enhanced expansion, persistence, and/or cytotoxicity compared to non-engineered cells of any kind.

In specific embodiments, the engineered cells are engineered specifically for the purpose of improving cell functionality (e.g., expansion, persistence, cytotoxicity, etc.) relative to non-engineered cells in an microenvironment (e.g., an acidic environment) comprising a pH of less than or equal to, exactly or about 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, or 5.0, or lower than 5.0. In specific embodiments, the engineered cells are engineered specifically for the purpose of improving cell functionality (e.g., expansion, persistence, cytotoxicity, etc.) relative to non-engineered cells in an acidic microenvironment comprising a low pH representative of increased levels of lactic acid relative to a non-acidic microenvironment. In specific embodiments, the engineered cells are engineered specifically for the purpose of improving cell functionality (e.g., expansion, persistence, cytotoxicity, etc.) relative to non-engineered cells in an acidic microenvironment comprising a low pH of less than or equal to, about or exactly 6.0. In specific embodiments, the engineered cells are engineered specifically for the purpose of improving cell functionality (e.g., expansion, persistence, cytotoxicity, etc.) relative to non-engineered cells in an acidic microenvironment comprising a low pH of less than or equal to, about or exactly 5.9. In certain embodiments, an acidic microenvironment comprises or is a Tumor Microenvironment (TME).

Any methods of treatment of the disclosure can be ameliorating, curative or prophylactic for the individual. It may be either part of an autologous immunotherapy or part of an allogeneic immunotherapy treatment. In specific cases, the methods are utilized for allogeneic immunotherapy, insofar as it enables the transformation of NK cells, typically obtained from donors, into non-alloreactive cells. This may be done under standard protocols and reproduced as many times as needed. The resultant engineered immune cells may be pooled and administered to one or several patients, being made available as an “off the shelf” therapeutic product. The cells may be stored, such as cryopreserved.

In some embodiments, administration of the composition(s) of the cells are for cancerous diseases of any kind, including tumorous diseases, including B cell malignancies, multiple myeloma, lung, brain, breast, blood, skin, pancreas, liver, colon, head and neck, kidney, thyroid, stomach, spleen, gall bladder, bone, ovary, testes, endometrium, prostate, rectum, anus, or cervix, for example. Exemplary indications for administration of the composition(s) of the cells are cancerous diseases, including any malignancies that express one or more of certain antigens associated with the cancer of an individual. The administration of the composition(s) of the disclosure is useful for all stages (I, II, III, and/or IV) and types of cancer, including for minimal residual disease, early cancer, advanced cancer, and/or metastatic cancer and/or refractory cancer, for example.

The disclosure further encompasses co-administration protocols with other compounds, e.g., bispecific antibody constructs, targeted toxins, or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Embodiments relate to a kit comprising constructs to produce the cells, a nucleic acid sequence as defined herein, a vector as defined herein and/or a host cell (such as an immune effector cell) as defined herein. It is also contemplated that the kit of this disclosure comprises a pharmaceutical composition as described herein above, either alone or in combination with further medicaments to be administered to an individual in need of medical treatment or intervention.

### V. Genetically Engineered Receptors

The immune cells of the present disclosure having reduced or inhibited expression of GPR4, GPR31, GPR68, GPR81, GPR132, GPR151, CREM, ICER, and/or CREB1 may be modified further to express one or more non-endogenous gene products. The gene product may or may not be a genetically engineered receptor. The receptor may be of any kind, including a receptor for an antigen, chemokine, or cytokine, for example. In cases wherein the receptor is for an antigen, the antigen may be a cancer antigen, including a solid tumor antigen.

The immune effector cells having reduced or inhibited expression of GPR4, GPR31, GPR68, GPR81, GPR132, GPR151, CREM, ICER, and/or CREB1 may be genetically engineered to express antigen receptors that target specific antigens, and such cells may be specifically designed to target one or more antigens that are present on cancer cells of an individual.

In specific embodiments, the immune effector cells comprising reduced or inhibited expression of GPR4, GPR31, GPR68, GPR81, GPR132, GPR151, CREM, ICER, and/or CREB1 may comprise an engineered antigen receptor, such as engineered TCRs or CARs. For example, the immune cells may be NK cells that are modified to express one or more CARs and/or TCRs having antigenic specificity for one or more specific antigens. In some aspects, the immune cells are engineered to express an antigen-specific CAR or antigen-specific TCR by knock-in of the CAR or TCR for example using CRISPR.

Suitable methods of modification are known in the art. See, for instance, Sambrook and Ausubel, supra. For example, the cells may be transduced to express a TCR having antigenic specificity for a cancer antigen using transduction techniques described in Heemskerk et al., 2008 and Johnson et al., 2009.

In some embodiments, the cells comprise one or more nucleic acids introduced via genetic engineering that encode one or more antigen receptors and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

### A. Chimeric Antigen Receptors

In some embodiments, the antigen-specific CAR comprises: a) one or more intracellular signaling domains, b) a transmembrane domain, and c) an extracellular domain comprising an antigen binding region that targets, including specifically binds, the desired antigen.

In some embodiments, the engineered antigen receptors include CARs, including activating or stimulatory CARs, costimulatory CARs (see WO2014/055668), and/or inhibitory CARs (iCARs, see e.g., Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising at least one intracellular signaling domain, a transmembrane domain, and an extracellular domain comprising one or more signaling motifs. In certain embodiments, the antigen-specific CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human antigen targeting CAR component may be encoded by nucleic acids derived from human genes. In some embodiments, such components may be used to enhance cellular immunotherapy for human patients. In a specific embodiment, the disclosure includes a full-length antigen-specific CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, DAP12, and 4-1BB (CD137). In addition to a primary signal initiated by CD35, an additional signal provided by a human costimulatory receptor inserted in a human CAR is important for full activation of NK cells and could help improve in vivo persistence and the therapeutic success of the adoptive immunotherapy.

In some embodiments, antigen-specific CAR is constructed with specificity for the antigen, such as the antigen being expressed on a normal or non-diseased cell type or on a diseased cell type. Thus, the CAR typically includes in its extracellular portion one or more antigen-binding molecules, such as one or more antigen-binding fragment, domain, or portion, or one or more antibody variable domains, and/or antibody molecules. In some embodiments, the antigen-specific CAR includes an antigen-binding portion or portions of an antibody molecule, such as a single-chain antibody fragment (scFv) derived from the variable heavy (V_{H}) and variable light (V_{L}) chains of a monoclonal antibody (mAb).

In certain embodiments, the antigen-specific CAR may be co-expressed with a cytokine to improve persistence when there is a low amount of tumor-associated antigen. For example, the CAR may be co-expressed with one or more cytokines, such as IL-7, IL-2, IL-15, IL-12, IL-18, IL-21, or a combination thereof.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD30, CD3 zeta, CD3 epsilon, CD3 gamma, CD3 delta, CD45, CD4, CD5, CD8, CD9, CD 16, CD22, CD33, CD37, CD64, CD80, CD86, CD134, CD137, CD154, ICOS/CD278, GITR/CD357, NKG2D, and DAP molecules. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the CD70-recognition domain to the cell surface, and, in some cases, (iv) artificial antigen presenting cells (aAPC) derived from K562 to be able to robustly and numerically expand CAR+immune cells (see e.g., Singh et al., 2008; Singh et al., 2011).

In certain embodiments, a CAR recognizes a TROP2 antigen. In certain embodiments, an anti-TROP2 CAR and/or an NK cell comprising the same is as described in International Patent Application Publication No. WO 2023/283644 A2, published on Jan. 12, 2023, which is incorporated herein by reference in its entirety.

In certain embodiments, a CAR recognizes a CD70 antigen. In certain embodiments, an anti-CD70 CAR comprises a CD27 derived polypeptide. In certain embodiments, an anti-CD70 CAR comprises, or is encoded by, a sequence at least or equal to, exactly or about 80%, 81%, 82%, 83%, 84%, 85%, 86%, 87%, 88%, 89%, 90%, 91%, 92%, 93%, 94%, 95%, 96%, 97%, 98%, 99%, or 100% identity to any one or more of SEQ ID NOs: 185-193. In certain embodiments, an anti-CD70 CAR and/or an NK cell comprising the same is as described in International Patent Application Publication No. WO 2021/142127 A1, published on Jul. 15, 2021, which is incorporated herein by reference in its entirety. In certain embodiments, an anti-CD70 CAR and/or an NK cell comprising the same is as described in International Patent Application Publication No. WO 2022/159791 A1, published on Jul. 28, 2022, which is incorporated herein by reference in its entirety. In certain embodiments, an anti-CD70 CAR and/or an NK cell comprising the same is as described in International Patent

Application Publication No. WO 2023/278520 A1, published on Jan. 5, 2023, which is incorporated herein by reference in its entirety.

SEQ ID NO: 185-Exemplary truncated CD27 amino
acid sequence
MARPHPWWLCVLGTLVGLSATPAPKSCPERHYWAQGKLCCQMCEPGTF
LVKDCDQHRKAAQCDPCIPGVSFSPDHHTRPHCESCRHCNSGLLVRNC
TITANAECACRNGWQCRDKECTECDPLPNPSLTARSSQALSPHPQPTH
LPYVSEMLEARTAGHMQTLADFRQLPARTLSTHWPPQRSLCSSDFIRF
WVLVVVGGVLACYSLLVTVAFIIFWV
SEQ ID NO: 186-Exemplary truncated CD27 amino
acid sequence
MARPHPWWLCVLGTLVGLSATPAPKSCPERHYWAQGKLCCQMCEPGTF
LVKDCDQHRKAAQCDPCIPGVSFSPDHHTRPHCESCRHCNSGLLVRNC
TITANAECACRNGWQCRDKECTECDPLPNPSLTARSSQALSPHPQPTH
LPYVSEMLEARTAGHMQTLADFRQLPARTLSTHWPPQRSLCSSDFIR
SEQ ID NO: 187-Exemplary CD28 TMD
FWVLVVVGGVLACYSLLVTVAFIIFWV
SEQ ID NO: 188, Exemplary full length WT CD27
amino acid sequence
MARPHPWWLCVLGTLVGLSATPAPKSCPERHYWAQGKLCCQMCEPGTF
LVKDCDQHRKAAQCDPCIPGVSFSPDHHTRPHCESCRHCNSGLLVRNC
TITANAECACRNGWQCRDKECTECDPLPNPSLTARSSQALSPHPQPTH
LPYVSEMLEARTAGHMQTLADFRQLPARTLSTHWPPQRSLCSSDFIRI
LVIFSGMFLVFTLAGALFLHQRRKYRSNKGESPVEPAEPCHYSCPREE
EGSTIPIQEDYRKPEPACSP
SEQ ID NO: 189, Exemplary truncated WT CD27
amino acid sequence
ATPAPKSCPERHYWAQGKLCCQMCEPGTFLVKDCDQHRKAAQCDPCIP
GVSFSPDHHTRPHCESCRHCNSGLLVRNCTITANAECACRNGWQCRDK
ECTECDPLPNPSLTARSSQALSPHPQPTHLPYVSEMLEARTAGHMQTL
ADFRQLPARTLSTHWPPQRSLCSSDFIR
SEQ ID NO: 190-Exemplary CD27 signal peptide
(SP) amino acid sequence
MARPHPWWLCVLGTLVGLS
SEQ ID NO: 191-Exemplary CD3z derived
Intracellular Signaling Domain (ICD) amino acid
sequence
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPRG
SEQ ID NO: 192-Exemplary CD28 derived
Intracellular Signaling Domain (ICD) amino acid
sequence
RSKRSRLLHSDYMNMTPRRPGPTRKHYQPYAPPRDFAAYRS
SEQ ID NO: 193-Exemplary DAP10 derived
Intracellular Signaling Domain (ICD) amino acid
sequence
LCARPRRSPAQEDGKVYINMPGRG

### B. T Cell Receptors (TCR)

In some embodiments, the genetically engineered antigen receptors include recombinant TCRs and/or TCRs cloned from naturally occurring T cells. A “T cell receptor” or “TCR” refers to a molecule that contains a variable a and β chains (also known as TCRα and TCRβ, respectively) or a variable y and 8 chains (also known as TCRγ and TCRδ, respectively) and that is capable of specifically binding to an antigen peptide bound to a major histocompatibility complex (MHC) receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to MHC molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or “antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (see e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5th ed.) at the N-terminus, and one constant domain (e.g., a-chain constant domain or Ca, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the a and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3δ chains, and a homodimer of CD3ζ chains. The CD3γ, CD3δ, and CD3δ chains are highly related cell surface proteins of the immunoglobulin superfamily containing a single immunoglobulin domain. The transmembrane regions of the CD3γ, CD3δ, and CD3δ chains are negatively charged, which is a characteristic that allows these chains to associate with the positively charged T cell receptor chains. The intracellular tails of the CD3γ, CD3δ, and CD3δ chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ -chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available sources. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

### VI. Cytokines

One or more cytokines may be utilized in immune effector cells having reduced or inhibited levels of expression of GPR4, GPR31, GPR68, GPR81, GPR132, GPR151, CREM, ICER, and/or CREB1. In some cases, one or more cytokines are present on the same vector molecule as the engineered receptor, although in other cases they are on separate molecules. In particular embodiments, one or more cytokines are co-expressed from the same vector as the engineered receptor. One or more cytokines may be produced as a separate polypeptide from the antigen-specific receptor. In some embodiments, an NK cell does not comprise one or more engineered receptors, but does comprise one or more heterologous cytokines. As one example, Interleukin-15 (IL-15), is utilized. IL-15 may be employed because, for example, it is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death. In certain embodiments, NK cells expressing IL-15 are capable of continued supportive cytokine signaling, which is useful for their survival post infusion. In addition to IL-15, other cytokines are envisioned. These include, but are not limited to, cytokines, chemokines, and other molecules that contribute to the activation and proliferation of cells used for human application. As one example, the cytokine is IL-15, IL-12, IL-2, IL-18, IL-21, IL-7, or a combination thereof. In certain embodiments, NK cells expressing IL-21 are capable of continued supportive cytokine signaling, which is useful for their survival post-infusion. In certain embodiments, a cytokine is expressed as part of a multicistronic construct with one or more functional proteins and/or marker proteins. Cells having reduced or inhibited levels of expression of GPR4, GPR31, GPR68, GPR81, GPR132, GPR151, CREM, ICER, and/or CREB1 and that may express one or more cytokines may be utilized and are capable of continued supportive cytokine signaling, which is useful for their survival post-infusion.

In specific embodiments, NK cells having reduced or inhibited level of expression of GPR4, GPR31, GPR68, GPR81, GPR132, GPR151, CREM, ICER, and/or CREB1 express one or more exogenously provided cytokines. The cytokine may be exogenously provided to the cells because it is expressed from an expression vector within the cell. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as one that expresses another gene product, such as a suicide gene. The cytokine may be expressed as a separate polypeptide molecule as a suicide gene and as a separate polypeptide from an engineered receptor of the cell. In some embodiments, the present disclosure concerns co-utilization of CAR and/or TCR vectors with IL-15, particularly in NK cells having reduced or inhibited levels of expression of GPR4, GPR31, GPR68, GPR81, GPR132, GPR151, CREM, ICER, and/or CREB1.

### VII. Suicide Genes

In particular embodiments, a suicide gene is utilized in conjunction with cell therapy of any kind to control its use and allow for termination of the cell therapy at a desired event and/or time. The suicide gene is employed in transduced cells for the purpose of eliciting death for the transduced cells when needed. The immune effector cells of the present disclosure that have been modified to harbor a vector encompassed by the disclosure may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidylate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be used. Other examples of suicide genes used with prodrug therapy are the E. coli cytosine deaminase gene and the HSV thymidine kinase gene.

Exemplary suicide genes also include CD20, CD52, EGFRv3, or inducible caspase 9. In one embodiment, a truncated version of EGFR variant III (EGFRv3) may be used as a suicide antigen that can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

In particular embodiments, vectors that encode the antigen-targeting CAR, or any vector in a NK cell encompassed herein, include one or more suicide genes. The suicide gene may or may not be on the same vector as an antigen-targeting CAR. In cases wherein the suicide gene is present on the same vector as the antigen-targeting CAR, the suicide gene and the CAR may be separated by an internal ribosome entry sites (IRES) elements or 2A element, for example.

In specific embodiments, the suicide gene is a tumor necrosis factor (TNF)-alpha mutant that is uncleavable by standard enzymes that cleave TNF in nature, such as TNF-alpha-converting enzyme (also referred to as TACE). As such, the TNF-alpha mutant is membrane-bound and nonsecretable, in particular embodiments. The TNF-alpha mutant used in the disclosure is targetable by one or more agents that bind the mutant, including at least an antibody, such that following binding of the agent(s) to the TNF-alpha mutant on the surface of the cell, the cell dies. Embodiments of the disclosure allow the TNF-alpha mutant to be utilized as a marker for cells that express it.

Cells expressing the uncleavable TNF-alpha mutants can be targeted for selective deletion including, for example, using FDA-approved TNF-α antibodies currently in the clinic, such as etanercept, infliximab or adalimumab. The mutated TNF-alpha polypeptide may be co-expressed with one or more therapeutic transgenes in the cell, such as a gene encoding a TCR or CAR, including CD70-targeting TCRs and/or CARs. In addition, the TNF-alpha mutant expressing cells have superior activity against the tumor target, mediated by the biological activity of the membrane-bound TNF-alpha protein.

With respect to wild-type, TNF-alpha has a 26 kD transmembrane form and a 17 kD secretory component. Some mutants described in Perez et al. (1990) may be utilized in the disclosure. In specific embodiments, examples of TNF-alpha mutants of the disclosure include at least the following with respect to the 17 kD TNF: (1) deletion of Vall and deletion of Prol12; (2) deletion of Val13; (3) deletion of Vall and deletion of Val13; (4) deletion of Val1 through and including Prol12 and deletion of Val13 (delete 13aa); (5) deletion of Ala-3 through to and including Val 13 (delete 14 aa). In specific embodiments, a TNF-alpha mutant comprises deletion of the respective amino acid at position −3,−2,−1, 1, 2, 3, 4, 5, 6, 7, 8, 9, 10, 11, 12, 13, or a combination thereof. Specific combinations include deletions at positions-3 through and including 13;-3 through and including 12;-3 through and including 11;-3 through and including 10;-3 through and including 9;-3 through and including 8;-3 through and including 7;-3 through and including 6;-3 through and including 5;-3 through and including 4;-3 through and including 3;-3 through and including 2;-3 through and including 1;-3 through and including-1;-3 through and including-2;-2 through and including 13;-2 through and including 12;-2 through and including 11;-2 through and including 10;-2 through and including 9;-2 through and including 8;-2 through and including 7;-2 through and including 6;-2 through and including 5;-2 through and including 4;-2 through and including 3;-2 through and including 2;-2 through and including 1;-2 through and including-1;-1 through and including 13;-1 through and including 12;-1 through and including 11;-1 through and including 10;-1 through and including 9;-1 through and including 8;-1 through and including 7;-1 through and including 6;-1 through and including 5;-1 through and including 4;-1 through and including 3;-1 through and including 2;-1 through and including 1; 1 through and including 13; 1 through and including 12; 1 through and including 11; 1 through and including 10; 1 through and including 9; 1 through and including 8; 1 through and including 7; 1 through and including 6; 1 through and including 5; 1 through and including 4; 1 through and including 3; 1 through and including 2; and so forth.

The TNF-alpha mutants may be generated by any suitable method, but in specific embodiments they are generated by site-directed mutagenesis. In some cases, the TNF-alpha mutants may have mutations other than those that render the protein uncleavable. In specific cases, the TNF-alpha mutants may have 1, 2, 3, or more mutations other than the deletions at Val1, Pro12, and/or Val13 or the region there between. The mutations other than those that render the mutants nonsecretable may be one or more of an amino acid substitution, deletion, addition, inversion, and so forth. In cases wherein the additional mutation is an amino acid substitution, the substitution may or may not be to a conservative amino acid, for example. In some cases, 1, 2, 3, 4, 5, or more additional amino acids may be present on the N-terminal and/or C-terminal ends of the protein. In some cases, a TNF-alpha mutant has (1) one or more mutations that render the mutant nonsecretable; (2) one or more mutations that prevents outside—in signaling for the mutant; and/or (3) one or more mutations that interfere with binding of the mutant to TNF Receptor 1 and/or TNF Receptor 2.

In particular embodiments, upon delivering an effective amount of one or more agents to bind to the TNF-alpha mutant-expressing antigen CAR-targeting cells, the majority of TNF-alpha mutant-expressing cells are eliminated. In specific embodiments, greater than 50%, 55%, 60%, 65%, 70%, 75%, 80%, 85%, 90%, 95%, 96%, 97%, 98%, or 99% of cells expressing the TNF-alpha mutants are eliminated in an individual. Following recognition of a need to eliminate the cells, the delivery of the agent(s) to the individual may continue until one or more symptoms are no longer present or until a sufficient number of cells have been eliminated. The cell numbers in the individual may be monitored using the TNF-alpha mutants as markers.

Embodiments of methods of the disclosure may comprise a first step of providing an effective amount of the cell therapy to an individual in need thereof, wherein the cells comprise one or more nonsecretable TNF-alpha mutants; and, a second step of eliminating the cells using the TNF-alpha mutant(s) as suicide genes (directly or indirectly through cell death by any mechanism). The second step may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin.

In additional embodiments, administration of one or more agents that bind the nonsecretable TNF-α during cytokine release syndrome or neurotoxicity, for example, have the added benefit of neutralizing the high levels of soluble TNF-alpha that contribute to the toxicity of the therapy. Soluble TNF-alpha is released at high levels during cytokine release syndrome and is a mediator of toxicity with CAR T-cell therapies. In such cases, the administration of TNF-alpha antibodies encompassed herein have a dual beneficial effect—i.e. selective deletion of the TNF-alpha mutant-expressing cells as well as neutralizing soluble TNF-alpha causing toxicity. Thus, embodiments of the disclosure encompass methods of eliminating or reducing the severity of cytokine release syndrome in an individual receiving, or who has received, adoptive cell therapy in which the cells express a nonsecretable TNF-alpha mutant, comprising the step of providing an effective amount of an agent that binds the nonsecretable TNF-alpha mutant, said agent causing in the individual (a) elimination of at least some of the cells of the cell therapy; and (b) reduction in levels of soluble TNF-alpha.

Embodiments of the disclosure include methods of reducing the effects of cytokine release syndrome in an individual that has received or who is receiving cell therapy with cells that express a nonsecretable TNF-alpha mutant, comprising the step of providing an effective amount of one or more agents that bind the mutant to cause in the individual (a) elimination of at least some of the cells of the cell therapy; and (b) reduction in the level of soluble TNF-alpha.

When the need arises for the TNF-alpha suicide gene to be utilized, the individual is provided an effective amount of one or more inhibitors that are able to inhibit, such as by binding directly, the TNF-alpha mutant on the surface of the cells. The inhibitor(s) may be provided to the individual systemically and/or locally in some embodiments. The inhibitor may be a polypeptide (such as an antibody), a nucleic acid, a small molecule (for example, a xanthine derivative), a peptide, or a combination thereof. In specific embodiments, the antibodies are FDA-approved. When the inhibitor is an antibody, the inhibitor may be a monoclonal antibody in at least some cases. When mixtures of antibodies are employed, one or more antibodies in the mixture may be a monoclonal antibody. Examples of small molecule TNF-alpha inhibitors include small molecules such as are described in U.S. Pat. No. 5,118,500, which is incorporated by reference herein in its entirety. Examples of polypeptide TNF-alpha inhibitors include polypeptides, such as those described in U.S. Pat. No. 6,143,866, which is incorporated by reference herein in its entirety.

In particular embodiments, at least one antibody is utilized to target the TNF-alpha mutant to trigger its activity as a suicide gene. Examples of antibodies include at least Adalimumab, Adalimumab-atto, Certolizumab pegol, Etanercept, Etanercept-szzs, Golimumab, Infliximab, Infliximab-dyyb, or a mixture thereof, for example.

Embodiments of the disclosure include methods of reducing the risk of toxicity of a cell therapy for an individual by modifying cells of a cell therapy to express a nonsecretable TNF-alpha mutant. The cell therapy is for cancer, in specific embodiments, and it may comprise an engineered receptor that targets an antigen, including a cancer antigen.

In particular embodiments, in addition to the inventive cell therapy of the disclosure, the individual may have been provided, may be provided, and/or may will be provided an additional therapy for the medical condition. In cases wherein the medical condition is cancer, the individual may be provided one or more of surgery, radiation, immunotherapy (other than the cell therapy of the present disclosure), hormone therapy, gene therapy, chemotherapy, and so forth.

Populations of cells having reduced or inhibited levels of expression of GPR4, GPR31, GPR68, GPR81, GPR132, GPR151, CREM, ICER, and/or CREB1 are provided at an effective level to an individual in need thereof. The cells may be administered to the individual by injection, intravenously, intraarterially, intraperitoneally, intratracheally, intratumorally, intramuscularly, endoscopically, intralesionally, intracranially, percutaneously, subcutaneously, regionally, by perfusion, in a tumor microenvironment, or a combination thereof.

In particular embodiments of the methods, the cells may be administered to the individual once or more than once. The duration of time between administrations of the cells to the individual may be 1-24 hours, 1-7 days, 1-4 weeks, 1-12 months, or 1 or more years.

### VIII. Vectors

In cases wherein the immune effector cell having reduced or inhibited level of expression of GPR4, GPR31, GPR68, GPR81, GPR132, GPR151, CREM, ICER, and/or CREB1 comprises a non-endogenous engineered gene product or exogenously provided gene product, the gene product may be delivered to the recipient immune effector cells by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, and so forth.

In cases wherein the immune cell is transduced with a vector encoding the antigen-targeting receptor and also requires transduction of another gene or genes into the cell, such as a suicide gene and/or cytokine and/or an optional therapeutic gene product, the antigen-targeting receptor, suicide gene, cytokine, and optional therapeutic gene may or may not be comprised on or with the same vector. In some cases, the antigen-targeting CAR, suicide gene, cytokine, and optional therapeutic gene are expressed from the same vector molecule, such as the same viral vector molecule. In such cases, the expression of the antigen-targeting CAR, suicide gene, cytokine, and optional therapeutic gene may or may not be regulated by the same regulatory element(s). When the antigen-targeting CAR, suicide gene, cytokine, and optional therapeutic gene are on the same vector, they may or may not be expressed as separate polypeptides. In cases wherein they are expressed as separate polypeptides, they may be separated on the vector by a 2A element or IRES element (or both kinds may be used on the same vector once or more than once), for example.

### A. General Embodiments

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure.

### 1. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells may be comprised of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters, for example. In cases wherein the vector is utilized for the generation of cancer therapy, a promoter may be effective under conditions of hypoxia.

### 2. Promoter/Enhancers

The expression constructs provided herein comprise a promoter to drive expression of the antigen receptor and other cistron gene products. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GAPDH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GenBank®, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

### 3. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As detailed elsewhere herein, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot- and-mouth disease virus 2A) or a “2A-like” sequence (e.g., Thosea asigna virus 2A; T2A) or porcine teschovirus-1 (P2A). In specific embodiments, in a single vector the multiple 2A sequences are non-identical, although in alternative embodiments the same vector utilizes two or more of the same 2A sequences. Examples of 2A sequences are provided in US 2011/0065779 which is incorporated by reference herein in its entirety.

### 4. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

### 5. Selection and Screenable Markers

In some embodiments, NK cells comprising a CD70-targeting receptor construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### B. Multicistronic Vectors

In particular embodiments, the antigen-targeting receptor, optional suicide gene, optional cytokine, and/or optional therapeutic gene are expressed from a multicistronic vector (The term “cistron” as used herein refers to a nucleic acid sequence from which a gene product may be produced). In specific embodiments, the multicistronic vector encodes the antigen-targeting receptor, the suicide gene, and at least one cytokine, and/or engineered receptor, such as a T-cell receptor and/or an additional non-antigen-targeting CAR. In some cases, the multicistronic vector encodes at least one antigen-targeting CAR, at least one TNF-alpha mutant, and at least one cytokine. The cytokine may be of a particular type of cytokine, such as human or mouse or any species. In specific cases, the cytokine is IL-15, IL-12, IL-2, IL-18, and/or IL-21.

In certain embodiments, the present disclosure provides a flexible, modular system (the term “modular” as used herein refers to a cistron or component of a cistron that allows for interchangeability thereof, such as by removal and replacement of an entire cistron or of a component of a cistron, respectively, for example by using standard recombination techniques) utilizing a polycistronic vector having the ability to express multiple cistrons at substantially identical levels. The system may be used for cell engineering allowing for combinatorial expression (including overexpression) of multiple genes. In specific embodiments, one or more of the genes expressed by the vector include one, two, or more antigen receptors. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise: (1) one or more reporters, for example fluorescent or enzymatic reporters, such as for cellular assays and animal imaging; (2) one or more cytokines or other signaling molecules; and/or (3) a suicide gene.

In specific cases, the vector may comprise at least 4 cistrons separated by cleavage sites of any kind, such as 2A cleavage sites. The vector may or may not be Moloney Murine

Leukemia Virus (MoMLV or MMLV)-based including the 3′ and 5′ LTR with the psi packaging sequence in a pUC19 backbone. The vector may comprise 4 or more cistrons with three or more 2A cleavage sites and multiple ORFs for gene swapping. The system allows for combinatorial overexpression of multiple genes (7 or more) that are flanked by restriction site(s) for rapid integration through subcloning, and the system also includes at least three 2A self-cleavage sites, in some embodiments. Thus, the system allows for expression of multiple CARs, TCRs, signaling molecules, cytokines, cytokine receptors, and/or homing receptors. This system may also be applied to other viral and non-viral vectors, including but not limited to lentivirus, adenovirus AAV, as well as non-viral plasmids.

The modular nature of the system also enables efficient subcloning of a gene into each of the 4 cistrons in the polycistronic expression vector and the swapping of genes, such as for rapid testing. Restriction sites strategically located in the polycistronic expression vector allow for swapping of genes with efficiency.

Embodiments of the disclosure encompass systems that utilize a polycistronic vector wherein at least part of the vector is modular, for example by allowing removal and replacement of one or more cistrons (or component(s) of one or more cistrons), such as by utilizing one or more restriction enzyme sites whose identity and location are specifically selected to facilitate the modular use of the vector. The vector also has embodiments wherein multiple of the cistrons are translated into a single polypeptide and processed into separate polypeptides, thereby imparting an advantage for the vector to express separate gene products in substantially equimolar concentrations.

The vector of the disclosure is configured for modularity to be able to change one or more cistrons of the vector and/or to change one or more components of one or more particular cistrons. The vector may be designed to utilize unique restriction enzyme sites flanking the ends of one or more cistrons and/or flanking the ends of one or more components of a particular cistron.

Embodiments of the disclosure include polycistronic vectors comprising at least two, at least three, or at least four cistrons each flanked by one or more restriction enzyme sites, wherein at least one cistron encodes for at least one antigen receptor. In some cases, two, three, four, or more of the cistrons are translated into a single polypeptide and cleaved into separate polypeptides, whereas in other cases multiple of the cistrons are translated into a single polypeptide and cleaved into separate polypeptides. Adjacent cistrons on the vector may be separated by a self-cleavage site, such as a 2A self-cleavage site. In some cases each of the cistrons expresses separate polypeptides from the vector. On particular cases, adjacent cistrons on the vector are separated by an IRES element.

In certain embodiments, the present disclosure provides a system for cell engineering allowing for combinatorial expression, including overexpression, of multiple cistrons that may include one, two, or more antigen receptors, for example. In particular embodiments, the use of a polycistronic vector as described herein allows for the vector to produce equimolar levels of multiple gene products from the same mRNA. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise one or more fluorescent or enzymatic reporters, such as for cellular assays and animal imaging. The vector may also comprise a suicide gene product for termination of cells harboring the vector when they are no longer needed or become deleterious to a host to which they have been provided.

In particular embodiments of the disclosure, at least one of the cistrons on the vector comprises two or more modular components, wherein each of the modular components within a cistron is flanked by one or more restriction enzyme sites. A cistron may comprise three, four, or five modular components, for example. In at least some cases, a cistron encodes an antigen receptor having different parts of the receptor encoded by corresponding modular components. A first modular component of a cistron may encode an antigen binding domain of the receptor. In addition, a second modular component of a cistron may encode a hinge region of the receptor. In addition, a third modular component of a cistron may encode a transmembrane domain of the receptor. In addition, a fourth modular component of a cistron may encode a first costimulatory domain. In addition, a fifth modular component of a cistron may encode a second costimulatory domain. In addition, a sixth modular component of a cistron may encode a signaling domain.

In particular aspects of the disclosure, two different cistrons on the vector each encode non-identical antigen receptors. Both antigen receptors may be encoded by a cistron comprising two or more modular components, including separate cistrons comprising two or more modular components. The antigen receptor may be a chimeric antigen receptor (CAR) and/or T cell receptor (TCR), for example.

In specific embodiments, the vector is a viral vector (retroviral vector, lentiviral vector, adenoviral vector, or adeno-associated viral vector, for example) or a non-viral vector. The vector may comprise a Moloney Murine Leukemia Virus (MMLV) 5′ LTR, 3′ LTR, and/or psi packaging element. In specific cases, the psi packaging is incorporated between the 5′ LTR and the antigen receptor coding sequence. The vector may or may not comprise pUC19 sequence. In some aspects of the vector, at least one cistron encodes for a cytokine (interleukin 15 (IL-15), IL-7, IL-21, or IL-2, for example), chemokine, cytokine receptor, and/or homing receptor.

When 2A cleavage sites are utilized in the vector, the 2A cleavage site may comprise a P2A, T2A, E2A and/or F2A site.

In addition to one cistron encoding a CD70-targeting CAR, any cistron of the vector may comprise a suicide gene. Any cistron of the vector may encode a reporter gene. In specific embodiments, a first cistron encodes a suicide gene, a second cistron encodes a CD70-targeting CAR, a third cistron encodes a reporter gene, and a fourth cistron encodes a cytokine. In certain embodiments, a first cistron encodes a suicide gene, a second cistron encodes a CD70-targeting CAR, a third cistron encodes a second CAR or another antigen receptor, and a fourth cistron encodes a cytokine. In specific embodiments, different parts of the a CD70-targeting CAR and/or another receptor are encoded by corresponding modular components and a first component of the second cistron encodes an antigen binding domain, a second component encodes a hinge and/or transmembrane domain, a third component encodes a costimulatory domain, and a fourth component encodes a signaling domain.

In specific embodiments, at least one of the cistrons encodes a suicide gene. In some embodiments, at least one of the cistrons encodes a cytokine. In certain embodiments, at least one cistron encodes an antigen-targeting CAR. A cistron may or may not encode a reporter gene. In certain embodiments, at least two cistrons encode two different antigen receptors (e.g., CARs and/or TCRs). A cistron may or may not encode a reporter gene.

In particular configurations of the genetic cargo of interest, a single vector may comprise a cistron that encodes an antigen-targeting CAR and a cistron that encodes a second antigen receptor that is non-identical to the antigen-targeting receptor. In specific embodiments, the first antigen receptor encodes an antigen-targeting CAR and the second antigen receptor encodes a TCR, or vice versa. In particular embodiments, a vector comprising separate cistrons that respectively encode an antigen-targeting CAR and a second antigen receptor also comprises a third cistron that encodes a cytokine or chemokine and a fourth cistron that encodes a suicide gene. However, the suicide gene and/or the cytokine (or chemokine) may not be present on the vector.

In particular embodiments, at least one cistron comprises multiple component(s) themselves that are modular. For example, one cistron may encode a multi-component gene product, such as an antigen receptor having multiple parts; in specific cases the antigen receptor is encoded from a single cistron, thereby ultimately producing a single polypeptide. The cistron encoding multiple components may have the multiple components separated by 1, 2, 3, 4, 5, or more restriction enzyme digestion sites, including 1, 2, 3, 4, 5, or more restriction enzyme digestion sites that are unique to the vector comprising the cistron. In specific embodiments, a cistron having multiple components encodes an antigen receptor having multiple corresponding parts each attributing a unique function to the receptor. In a specific embodiment, each or the majority of components of the multi-component cistrons is separated by one or more restriction enzyme digestion sites that are unique to the vector, allowing the interchangeability of separate components when desired.

In specific embodiments, each component of a multi-component cistron corresponds to a different part of an encoded antigen receptor, such as an antigen-targeting CAR. In illustrative embodiments, component 1 may encode an antigen-binding domain of the receptor; component 2 may encode a hinge domain of the receptor; component 3 may encode a transmembrane domain of the receptor; component 4 may encode a costimulatory domain of the receptor, and component 5 may encode a signaling domain of the receptor. In specific embodiments, an antigen-targeting CAR may comprise one or more costimulatory domains, each separated by unique restriction enzyme digestion sites for interchangeability of the costimulatory domain(s) within the receptor.

In specific embodiments, there is a polycistronic vector having four separate cistrons where adjacent cistrons are separated by a 2A cleavage site, although in specific embodiments instead of a 2A cleavage site there is an element that directly or indirectly causes separate polypeptides to be produced from the cistrons (such as an IRES sequence). For example, four separate cistrons may be separated by three 2A peptide cleavage sites, and each cistron has restriction sites (X1, X2, etc.) flanking each end of the cistron to allow for interchangeability of the particular cistron, such as with another cistron or other type of sequence, and upon using standard recombination techniques. In specific embodiments, the restriction enzyme site(s) that flank each of the cistrons is unique to the vector to allow ease of recombination, although in alternative embodiments the restriction enzyme site is not unique to the vector.

In particular embodiments, the vector provides for a unique, second level of modularity by allowing for interchangeability within a particular cistron, including within multiple components of a particular cistron. The multiple components of a particular cistron may be separated by one or more restriction enzyme sites, including those unique to the vector, to allow for interchangeability of one or more components within the cistron. As an example, cistron 2 may comprise five separate components, although there may be 2, 3, 4, 5, 6, or more components per cistron. As an example, a vector may include cistron 2 that has five components each separated by unique enzyme restriction sites X9, X10, X11, X12, X13, and X14, to allow for standard recombination to exchange different components 1, 2, 3, 4, and/or 5. In some cases, there may be multiple restriction enzyme sites between the different components (that are unique, although alternatively one or more are not unique) and there may be sequence in between the multiple restriction enzyme sites (although alternatively there may not be). In certain embodiments, all components encoded by a cistron are designed for the purpose of being interchangeable. In particular cases, one or more components of a cistron are designed to be interchangeable, whereas one or more other components of the cistron may not be designed to be interchangeable.

In specific embodiments, a cistron encodes an antigen-targeting CAR molecule having multiple components. For example, cistron 2 may be comprised of sequence that encodes an antigen-targeting CAR molecule having its separate components represented by component 1, component 2, component 3, etc. The CAR molecule may comprise 2, 3, 4, 5, 6, 7, 8, or more interchangeable components. In a specific example, component 1 encodes a scFv; component 2 encodes a hinge; component 3 encodes a transmembrane domain; component 4 encodes a costimulatory domain (although there may also be component 4′ that encodes a second or more costimulatory domain flanked by restriction sites for exchange); and component 5 encodes a signaling domain. In a particular example, component 1 encodes an scFv; component 2 encodes an IgG1 hinge and/or transmembrane domain; component 3 encodes CD28; and component 4 encodes CD3 zeta.

One of skill in the art recognizes in the design of the vector that the various cistrons and components must be configured such that they are kept in frame when necessary.

In a particular example, cistron 1 encodes a suicide gene; cistron 2 encodes an antigen-targeting CAR; cistron 3 encodes a reporter gene; cistron 4 encodes a cytokine; component 1 of cistron 2 encodes an scFv; component 2 of cistron 2 encodes IgG1 hinge; component 3 of cistron 2 encodes CD28; and component 4 encodes CD3 zeta.

A restriction enzyme site may be of any kind and may include any number of bases in its recognition site, such as between 4 and 8 bases; the number of bases in the recognition site may be at least 4, 5, 6, 7, 8, or more. The site when cut may produce a blunt cut or sticky ends. The restriction enzyme may be of Type I, Type II, Type III, or Type IV, for example. Restriction enzyme sites may be obtained from available databases, such as Integrated relational Enzyme database (IntEnz) or BRENDA (The Comprehensive Enzyme Information System).

Exemplary vectors may be circular and by convention, where position 1 (12 o'clock position at the top of the circle, with the rest of the sequence in clock-wise direction) is set at the start of 5′ LTR.

In embodiments wherein self-cleaving 2A peptides are utilized, the 2A peptides may be 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot- and-mouth disease virus), after which E2A (equine rhinitis A virus), P2A (porcine teschovirus-1 2A), and T2A (Thosea asigna virus 2A) were also identified. The mechanism of 2A-mediated “self-cleavage” was discovered to be ribosome skipping the formation of a glycyl-prolyl peptide bond at the C-terminus of the 2A.

In specific cases, the vector may be a γ-retroviral transfer vector. The retroviral transfer vector may comprise a backbone based on a plasmid, such as the pUC19 plasmid (large fragment (2.63 kb) in between HindIII and EcoRI restriction enzyme sites). The backbone may carry viral components from Moloney Murine Leukemia Virus (MoMLV) including 5′ LTR, psi packaging sequence, and 3′ LTR. LTRs are long terminal repeats found on either side of a retroviral provirus, and in the case of a transfer vector, bracket the genetic cargo of interest, such as antigen-targeting CARs and associated components. The psi packaging sequence, which is a target site for packaging by nucleocapsid, is also incorporated in cis, sandwiched between the 5′ LTR and the CAR coding sequence. Thus, the basic structure of an example of a transfer vector can be configured as such: pUC19 sequence-5′ LTR-psi packaging sequence-genetic cargo of interest-3′ LTR-pUC19 sequence. This system may also be applied to other viral and non-viral vectors, including but not limited to lentivirus, adenovirus AAV, as well as non-viral plasmids.

### A. Pharmaceutical Compositions

Also provided herein are pharmaceutical compositions and formulations comprising transduced NK cells and a pharmaceutically acceptable carrier. The transduced cells may be comprised in a media suitable for transfer to an individual and/or media suitable for preservation, such as cryopreservation, including prior to transfer to an individual.

Pharmaceutical compositions and formulations as described herein can be prepared by mixing the active ingredients (such as the cells) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### B. Combination Therapies

In certain embodiments, the compositions and methods of the present embodiments involve an immune cell population (including NK cell population) in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or cell therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegal1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel; gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabine, navelbine, farnesyl-protein tansferase inhibitors, transplatinum, and pharmaceutically acceptable salts, acids, or derivatives of any of the above.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (see e.g., Leah et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12 (4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure.

For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95 (17): 10067-10071; Camacho et al. (2004) J Clin Oncology 22 (145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and YERVOY®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the V_{H} region of ipilimumab, and the CDR1, CDR2 and CDR3 domains of the V_{L} region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. U.S. Pat. Nos. 5,844,905, 5,885,796 and International Patent Application Nos. WO1995001994 and WO1998042752; all incorporated herein by reference, and immunoadhesins such as described in U.S. Patent No. U.S. Pat. No. 8,329,867, incorporated herein by reference.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IX. Kits of the Disclosure

Any of the compositions described herein may be comprised in a kit. In a non-limiting example, cells that have reduced or inhibited levels of expression of GPR4, GPR31, GPR68, GPR81, GPR132, GPR151, CREM, ICER, and/or CREB1, reagents to produce the cells, vectors, and reagents to produce vectors and/or components thereof may be comprised in a kit. In certain embodiments, NK cells may be comprised in a kit, and they may or may not yet be modified in any manner. Such a kit may or may not have one or more reagents for manipulation of cells. Such reagents include small molecules, proteins, nucleic acids, antibodies, buffers, primers, nucleotides, salts, and/or a combination thereof, for example. Nucleotides that encode CRISPR reagents to KO GPR4, GPR31, GPR68, GPR81, GPR132, GPR151, CREM, ICER, and/or CREB1, suicide gene products, receptors, and/or cytokines may be included in the kit. Proteins, such as cytokines or antibodies, including monoclonal antibodies, may be included in the kit. Nucleotides that encode components of engineered CAR receptors or TCR receptors may be included in the kit, including reagents to generate same.

In particular aspects, the kit comprises the NK cell therapy of the disclosure and also another cancer therapy. In some cases, the kit, in addition to the cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

The kits may comprise suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow-molded plastic containers into which the desired vials are retained.

### X. EXAMPLES

The following examples are included to demonstrate certain non-limiting aspects of the disclosure. It should be appreciated by those of skill in the art that the techniques disclosed in the examples that follow represent techniques discovered by the inventors to function well in the practice of the disclosed subject matter. However, those of skill in the art should, in light of the present disclosure, appreciate that many changes can be made in the specific embodiments that are disclosed and still obtain a like or similar result without departing from the spirit and scope of the disclosed subject matter.

### Example 1-Proton-Sensing Gprs and Crem were Highly Expressed in Tumor-Infiltrating Immune Cells of Glycolytic Tumors

The single cell RNA sequencing (scRNA seq) data of tumor samples derived from patients with pancreatic cancer were interrogated for the expression of genes encoding enzymes involved in glycolysis. The inventors determined that tumor cells have significant upregulation of glycolytic genes (FIG. 1A). This reflects active aerobic and anaerobic glycolysis leading to organic and inorganic acid accumulation in the tumor microenvironment (TME), as has been previously shown in the literature for multiple tumor types. Next, the expression of proton-sensing G-protein coupled receptors (GPRs) such as GPR68 and GPR132 was determined, as was the expression of downstream signaling mediators of response to acidity in the cAMP pathway such as CREM and CREB1, for the corresponding tumor-infiltrating NK cells. A number of these genes were found to be significantly upregulated in NK cells within the TME compared to NK cells from the peripheral blood (FIG. 1B). As CREM is a key transcription factor in the CAMP pathway, exhibited a significant upregulation in NK cells in the TME, and was implicated in mediating common downstream signaling of these proton-sensing GPRs, the CREM gene was determined to be a potential target for genetic modulation to potentially counteract acidity-mediated immunosuppression.

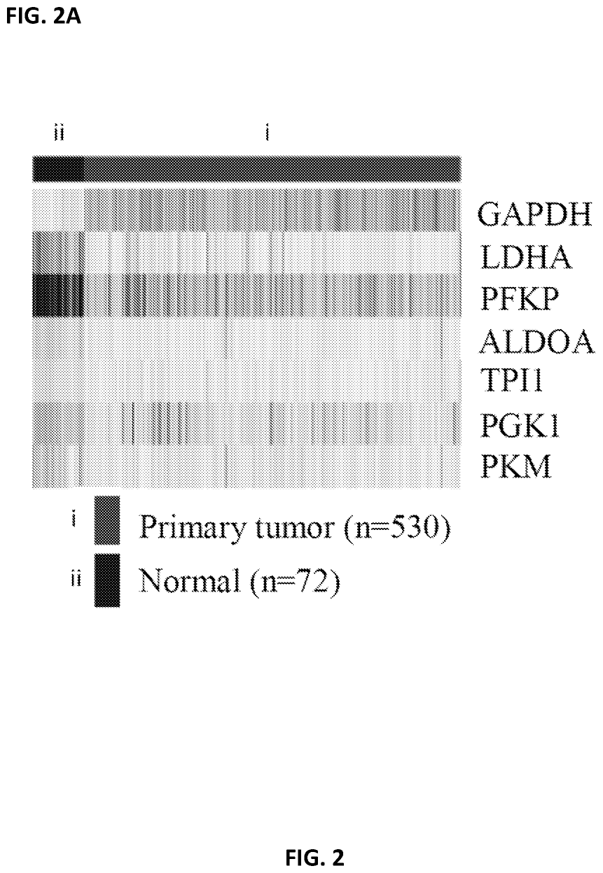

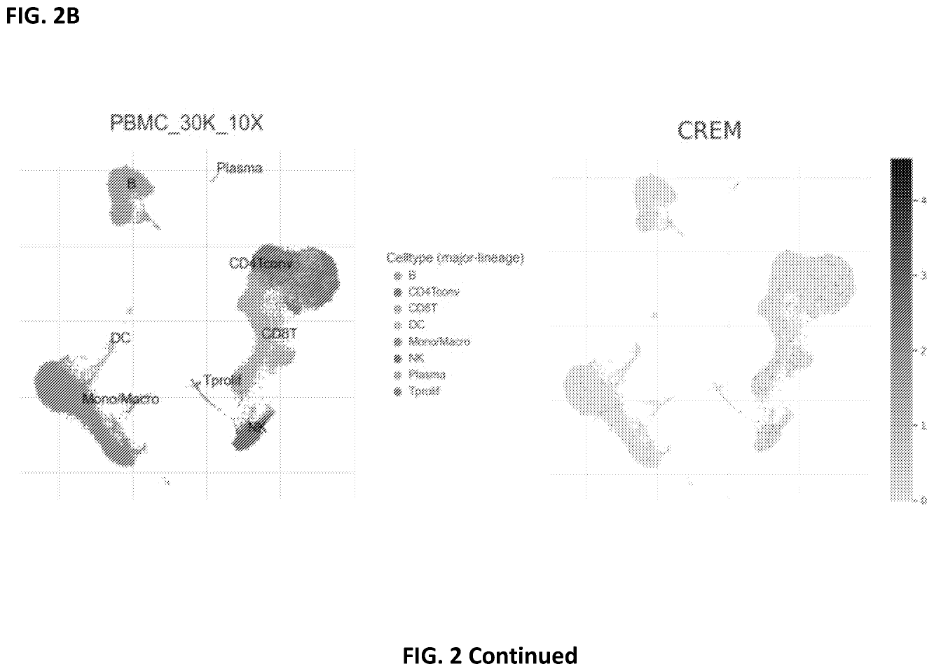

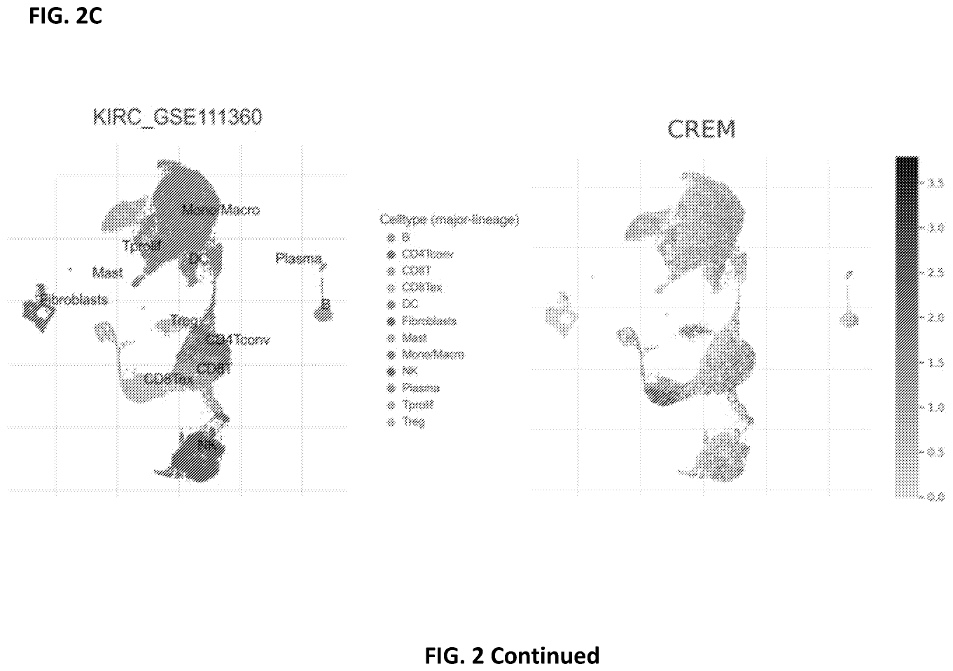

The expression of CREM on tumor-infiltrating immune cells in clear cell renal cell carcinoma (CC-RCC) was interrogated due to this tumor's well established “Warburg effect” and highly glycolytic state (see e.g., Courtney K. D. et al., 2018; as it relates to von Hippel-Lindau (VHL) gene loss, which mechanistically drives the development of the majority of CC-RCC) and as confirmed by high expression of glycolytic enzymes (FIG. 2A, from TCGA data). Publicly available scRNA seq data from the TISCH database was used to compare the expression of CREM in kidney cancer-infiltrating immune cells vs. peripheral blood immune cells from healthy donors (see e.g., Sun D. et al., 2020). As shown in FIG. 2B, a significant upregulation of CREM in various immune cells in the TME was observed compared to rare expression in immune cells in the peripheral blood from healthy donors.

### Example 2-Endonuclease Mediated Knockout of Endogenous Genes

Guide RNAs (gRNAs) were designed that induce double-stranded breaks in GPR4, GPR31, GPR68, GPR81, GPR132, GPR151, CREM, ICER, or CREB1, while having both high on-target and low off-target activity scores. Exemplary crispr RNA (crRNA) sequences suitable for incorporation into a gRNA are provided in Table 2.

TABLE 2
Exemplary crRNA sequences

| | | Sequence |
|---|---|---|
| | crRNA Sequences | Identity |
| Gene | (5′ to 3′) | Number |
| GPR4 | AGCCCACGAACACCCGATAG | 3 |
| GPR4 | CATCTCGGTGGACCGCTACC | 4 |
| GPR4 | TACATCTTTGTCATCGGCGT | 5 |
| GPR4 | GGTCTCGGAAGAGCTCGTCA | 6 |
| GPR4 | ACTTCCTGCACCACGACAAC | 7 |
| GPR4 | GACCACGGAGCTCACGGCCA | 8 |
| GPR4 | ATGGGCAACCACACGTGGGA | 9 |
| GPR4 | AGGAAGTAGTCCACCCACAG | 10 |
| GPR4 | AACCTCTATCGGGTGTTCGT | 11 |
| GPR4 | CGCCGATGACAAAGATGTAG | 12 |
| GPR4 | CACGGAGCTGGGCGCCAACT | 13 |
| GPR4 | CACCACGACAACTGGATCCA | 14 |
| GPR4 | TGGCCTGGATGAACCTCTAT | 15 |
| GPR4 | GACAACTGGATCCACGGCCC | 16 |
| GPR4 | ACAACTGGATCCACGGCCCC | 17 |
| GPR4 | GGAAGAGCTCGTCATGGAAC | 18 |
| GPR4 | GGTCCACCGAGATGCAGCAC | 19 |
| GPR4 | GCATGAGCGCCCACGGGAAG | 20 |
| GPR4 | GAAGAGCTCGTCATGGAACA | 21 |
| GPR4 | GATGAACCCAAAGAGCTTGC | 22 |
| GPR4 | GGACAGCAAGAGCACGTGAT | 23 |
| GPR4 | GACAGCAAGAGCACGTGATA | 24 |
| GPR4 | TTTCACCAGCCTCAACTGTG | 25 |
| GPR31 | GCAGGTTGACCTTAAGCCGA | 26 |
| GPR31 | GTACCGGTCCAAAGCCACGG | 27 |
| GPR31 | TCCACACCCTGACCCGGAAC | 28 |
| GPR31 | GGTGCCACAGTTTCTACTCC | 29 |
| GPR31 | CTGCATTGCAGAACACGATG | 30 |
| GPR31 | TGTGGCACCTGGTGGAGTTC | 31 |
| GPR31 | AGAGATGAGCAAGCCCGGGC | 32 |
| GPR31 | AGCAGGTTGACCTTAAGCCG | 33 |
| GPR31 | GCCGAGGGTGGACCACACGG | 34 |
| GPR31 | GGTGGACCACACGGAGGTAC | 35 |
| GPR31 | ACCTCCGTGTGGTCCACCCT | 36 |
| GPR31 | TAAGCCGAGGGTGGACCACA | 37 |
| GPR31 | ACGGCTTCCACACCCTGACC | 38 |
| GPR31 | GAGGTACCGGTCCAAAGCCA | 39 |
| GPR31 | GGTCAACCTGCTGTCTCCTC | 40 |
| GPR31 | CAGAACACGATGAGGCCAAA | 41 |
| GPR31 | GGAAGGCCATCCCCACGCTG | 42 |
| GPR68 | GATGACCACGCTGACGCCGA | 43 |
| GPR68 | CACGGTAACATAGACCACCG | 44 |
| GPR68 | ACGTGCTGCAGCACGACAAC | 45 |
| GPR68 | GATCAAGGCCCGGAACGAGC | 46 |
| GPR68 | GAGAACATCTACATCAGCGT | 47 |
| GPR68 | GGTAGTGCTCAAAGCACACG | 48 |
| GPR68 | CATCTACTTCCTGATGCACG | 49 |
| GPR68 | GCCGCAAGGACCAGATCCAG | 50 |
| GPR68 | CAACTACTACCGCTTCCTGG | 51 |
| GPR68 | AACTGCCTGTCCCTCTACTT | 52 |
| GPR68 | GCACGGTAACATAGACCACC | 53 |
| GPR68 | CCATACCATCCACCAGACGC | 54 |
| GPR68 | AGAAGAGGTCGGCCACCGTC | 55 |
| GPR68 | CAGCACGACAACTGGTCTCA | 56 |
| GPR68 | GTACCTGTGCAACCTGACGG | 57 |
| GPR68 | AGCACGGTAACATAGACCAC | 58 |
| GPR68 | CTACTTCCTGATGCACGAGG | 59 |
| GPR68 | CTTTGAGCACTACCCCATCC | 60 |
| GPR68 | CGTGTACCTGTGCAACCTGA | 61 |
| GPR68 | CCACCAGTTCCGGACCCTGA | 62 |
| GPR68 | CGGGAAGCCCACCACCAGCA | 63 |
| GPR68 | TGATGTAGATGTTCTCGTAC | 64 |
| GPR68 | CCAGTTCCGGACCCTGAAGG | 65 |
| GPR68 | GAAGGGGCCACACTCCTCAT | 66 |
| GPR68 | CAAGGACCAGATCCAGCGGC | 67 |
| GPR68 | GCCCTACCACGTGTTGCTGC | 68 |
| GPR68 | CGCAGTTGAAGCTGGTGAGC | 69 |
| GPR68 | TCGTCAGCGAGACCACCCAC | 70 |
| GPR68 | CGTCAGCGAGACCACCCACC | 71 |
| GPR68 | CGAGACCACCCACCGGGACC | 72 |
| GPR68 | GCTGTTGACCAAGCTCCACC | 73 |
| GPR68 | GTGGAGCTTGGTCAACAGCT | 74 |
| GPR81 | GAGCATCGTGTTCCTTACGG | 75 |
| GPR81 | CGTGAAGAGCCCCACTCGGC | 76 |
| GPR81 | CGGTGAACACTATCTCCACC | 77 |
| GPR81 | TTCATCATGGAGTCGGCCAA | 78 |
| GPR81 | AGGTAATAGTCTGTCCGAAA | 79 |
| GPR81 | TGCCACCACCATGATGAACC | 80 |
| GPR81 | CAGCCACGGCCAAATTGAAA | 81 |
| GPR81 | CTGGAGTTCTTTATGCCCCT | 82 |
| GPR81 | GCAAAAGATACACTGTTCCC | 83 |
| GPR81 | AAGGCCACAATGAGCAGCGG | 84 |
| GPR81 | CGTAAGGAACACGATGCTCC | 85 |
| GPR81 | CCGAGTGGGGCTCTTCACGT | 86 |
| GPR81 | GAACACTATCTCCACCCGGG | 87 |
| GPR81 | ATTACCTCAGACGTAGACAC | 88 |
| GPR81 | GGTGAACACTATCTCCACCC | 89 |
| GPR81 | CACTATCTCCACCCGGGTGG | 90 |
| GPR81 | CCAACGTGAAGAGCCCCACT | 91 |
| GPR81 | TTACCTCAGACGTAGACACT | 92 |
| GPR81 | GTGAAGAGCCCCACTCGGCA | 93 |
| GPR81 | AGTGGTCCACCCCCACCACG | 94 |
| GPR132 | TACTACGCCAGGTTCACCGT | 95 |
| GPR132 | CCGCGCTGTACACCACGACC | 96 |
| GPR132 | CGTACACCACGGCCACGAAG | 97 |
| GPR132 | GGTGAAGCACTCGGCCATCG | 98 |
| GPR132 | GGTAGTGAACGATCCCGACG | 99 |
| GPR132 | CTCTGCGAGCTGCTGTACAC | 100 |
| GPR132 | AGTGACCACCACTGCCCCGT | 101 |
| GPR132 | GGTACTGCAGGGCAACGTGC | 102 |
| GPR132 | TCACCAACCACCGGATTTTC | 103 |
| GPR132 | GATGTAGGCGGTCACCTTGC | 104 |
| GPR132 | TTCGAAGGACACGTTGTTGC | 105 |
| GPR132 | GAAGATGACAACCACCGCGA | 106 |
| GPR132 | ACGTGTCCTTCGAAGAGAGC | 107 |
| GPR132 | GACTATCCTGCTCTCTTCGA | 108 |
| GPR132 | TCACTACCCGGTGTTCCAGA | 109 |
| GPR132 | GGACACGTTGTTGCAGGTCT | 110 |
| GPR132 | GATGGCCGAGTGCTTCACCT | 111 |
| GPR132 | TCATCGCCTTCACCAACCAC | 112 |
| GPR132 | TGTCTTCCGTCTGGAACACC | 113 |
| GPR132 | TTGTCTTCCGTCTGGAACAC | 114 |
| GPR132 | CCTGTCCACGGTGAACGGCG | 115 |
| GPR132 | GAAGTGTCCAGAATCCATAA | 116 |
| GPR132 | TGTCCAGAATCCATAAGGGG | 117 |
| GPR132 | CCATGAAGACAGACGTCACC | 118 |
| GPR132 | GGTGACCCTGGTGGGTGCAC | 119 |
| GPR151 | TGCTAAAGAACCATTCCGGC | 120 |
| GPR151 | GGTACCCTCCGGCAAAGTGG | 121 |
| GPR151 | TGGCCACCACAACGATTGTC | 122 |
| GPR151 | GGCGTACTCCAAAAGTGTTT | 123 |
| GPR151 | CCACAGCTGGTACATCCACG | 124 |
| GPR151 | GTTGTGGATACTCACTTGCT | 125 |
| GPR151 | GGACTGGAGAACCATCATCC | 126 |
| GPR151 | TGGTAAGCTCTACCCACTCC | 127 |
| GPR151 | TAGTTCCTCGTTTTTTACAT | 128 |
| GPR151 | CTCCTTCACAATGCTTGGAA | 129 |
| GPR151 | GCTAAAGAACCATTCCGGCA | 130 |
| GPR151 | TTCTGCACCTATCCGAGCTA | 131 |
| GPR151 | CTAAAGAACCATTCCGGCAG | 132 |
| GPR151 | CACAACGATTGTCAGGCTCT | 133 |
| GPR151 | CAAGAGCCTGACAATCGTTG | 134 |
| GPR151 | GTATCCACAACTACACCATC | 135 |
| GPR151 | CTCACCTCCACTTTGCCGGA | 136 |
| GPR151 | CCTCGTGGATGTACCAGCTG | 137 |
| GPR151 | CTGACCAGATGGTGTAGTTG | 138 |
| GPR151 | TGGATACTCACTTGCTTGGC | 139 |
| CREM | ACCACCTAGTATTGCTACCA | 140 |
| CREM | TATAAGAGGGTCTTCGTGAA | 141 |
| CREM | TCTTCAATCTTGGGAACACC | 142 |
| CREM | CTAGTCTGATATATGCTAGT | 143 |
| CREM | GCCATGGTAGCAATACTAGG | 144 |
| CREM | TATGCTAGTTGGTACTGCCA | 145 |
| CREM | ACTGCCATGGTAGCAATACT | 146 |
| CREM | CAACACGCAAACGAGAGCTG | 147 |
| CREM | CCAGAACAAGAAGCTTATAG | 148 |
| CREM | GCATATATCAGACTAGCACG | 149 |
| CREM | GCTCATGTGCAGACTCAGAC | 150 |
| CREM | GTAAGTTGGCATGTCACCAG | 151 |
| CREM | GGTGTATCCTCAGCACGGAG | 152 |
| CREM | GGACTTCCAGCACTGCAACT | 153 |
| CREM | CCTACTGCTGCTTTGCCACA | 154 |
| CREM | TAGCATATATCAGACTAGCA | 155 |
| CREM | TCCTACTGCTGCTTTGCCAC | 156 |
| CREM | TTTCAGGCGTCCTATAGAAG | 157 |
| CREM | GAACTGTCCTCTGATGTGCC | 158 |
| CREM | GTCTGGGTGTATCCTCAGCA | 159 |
| CREM | AAGCTGCCCGGGAGTGTCGC | 160 |
| CREM | TGATGGTGTTCAGGGACTGC | 181 |
| CREM | ACTGGTGACATGCCAACTTAC | 182 |
| ICER | CTGTCTGCAGAAGCCCATTA | 161 |
| ICER | GCTGTAACTGGAGATGACAC | 183 |
| ICER | GCTTAGTGGTAAGATCGAGC | 184 |
| CREB1 | CTGGCTAACAATGGTACCGA | 162 |
| CREB1 | CAGCTGTACTAGAGTTACGG | 163 |
| CREB1 | TGGAGTTGGCACCGTTACAG | 164 |
| CREB1 | TACTGTAGAATGGTAGTACC | 165 |
| CREB1 | GACTTATCTTCTGATGCACC | 166 |
| CREB1 | TAACTGATTCCCAAAAGCGA | 167 |
| CREB1 | GTTCTCGGCTCCAGATTCCA | 168 |
| CREB1 | GGGCTTGAACTGTCATTTGT | 169 |
| CREB1 | TCAAGTCCATGGAGTCATTC | 170 |
| CREB1 | TGGCTAACAATGGTACCGAT | 171 |
| CREB1 | GGCTAACAATGGTACCGATG | 172 |
| CREB1 | AGCTGTACTAGAGTTACGGT | 173 |
| CREB1 | TATACTGTCCACTGCTAGTT | 174 |
| CREB1 | CTCTAGTACAGCTGCCCAAT | 175 |
| CREB1 | GACTTGAACTGTCTGCCCAT | 176 |
| CREB1 | ACTCTAGTACAGCTGCCCAA | 177 |
| CREB1 | TCTTCAATCCTTGGCACTCC | 178 |
| CREB1 | ACTTGAACTGTCTGCCCATT | 179 |
| CREB1 | CCACTGCTAGTTTGGTAAAT | 180 |

For each of the gene candidates for genetic targeting in immune effector cells, a panel of crispr RNA (crRNA) was designed (see Table 2), the crRNA were complexed with tracr RNA (the binding scaffold for the Cas nuclease) to form a guide RNA (gRNA).

Any one or more of the gene candidates for genetic targeting in immune effector cells are knocked out and/or otherwise mutated by one or more crRNA complexed with tracr RNA to form a guide RNA.

### Example 3-Crem Ko Significantly Improved the Cytotoxicity Of Nk Cells in an Acidic Environment and the Tme

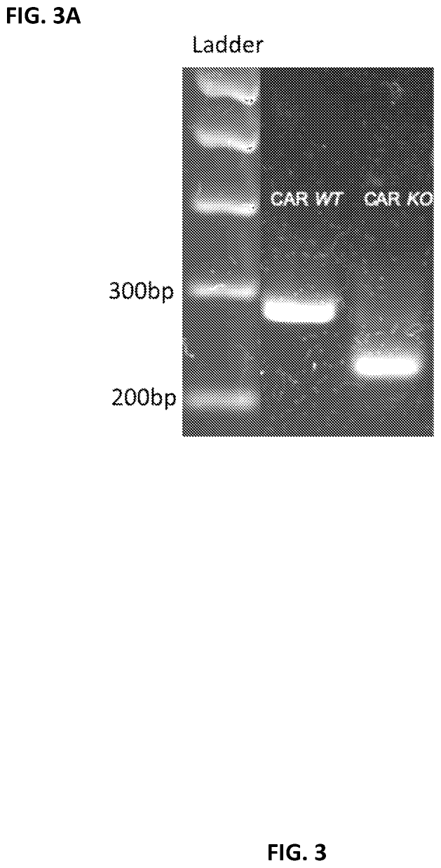

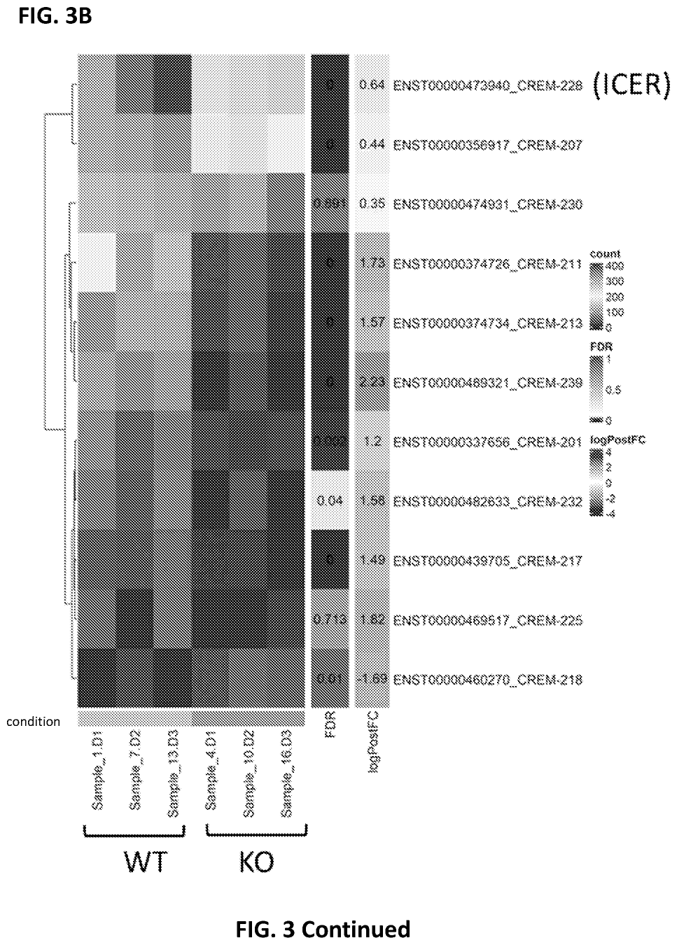

As described above, CREM was found to be consistently upregulated in TME-infiltrating immune cells across various glycolytic tumor models. As CREM protein is a key transcription factor in the cAMP pathway, the inventors created knock-out (KO) CREM loci in NK cells. The CREM loci KO acted as a proof-of-principle for targeting the CAMP pathway activation in response to acidity in an attempt to abrogate acidity-mediated immunosuppression. Using the CRISPR-Cas9 system with SEQ ID NO: 140 and 142, CREM KO loci in NK cells were efficiently and stably created (FIGS. 3A and 3B). FIG. 3B shows selected CREM isoforms from bulk RNA sequencing data obtained from CREM WT vs. CREM KO NK cells derived from 3 cord blood donors (D1, D2, and D3). The heatmap color scale was representative of the counts of expressing cells from 0 (dark blue) to 400 (red); the FDR column corresponds to the false discovery rate (significance <0.05); while the logPostFC column represents the log of the fold change of WT vs. KO expression levels, positive values indicated that RNA isoform expression is higher in CREM WT NK cells and negative values indicated that RNA isoform expression is higher in CREM KO NK cells. The RNA isoform expression analysis demonstrated that multiple CREM isoforms in mutated NK cells were effectively knocked out and/or significantly knocked down. Examples of knocked out and/or significantly knocked down RNA isoforms included: the ICER isoform (CREM-228), CREM-207, CREM-211, CREM-213, CREM-239, CREM-201, CREM-232, and CREM-217. RNA isoform CREM-218 was found to be upregulated in CREM KO NK cells relative to CREM WT NK cells.

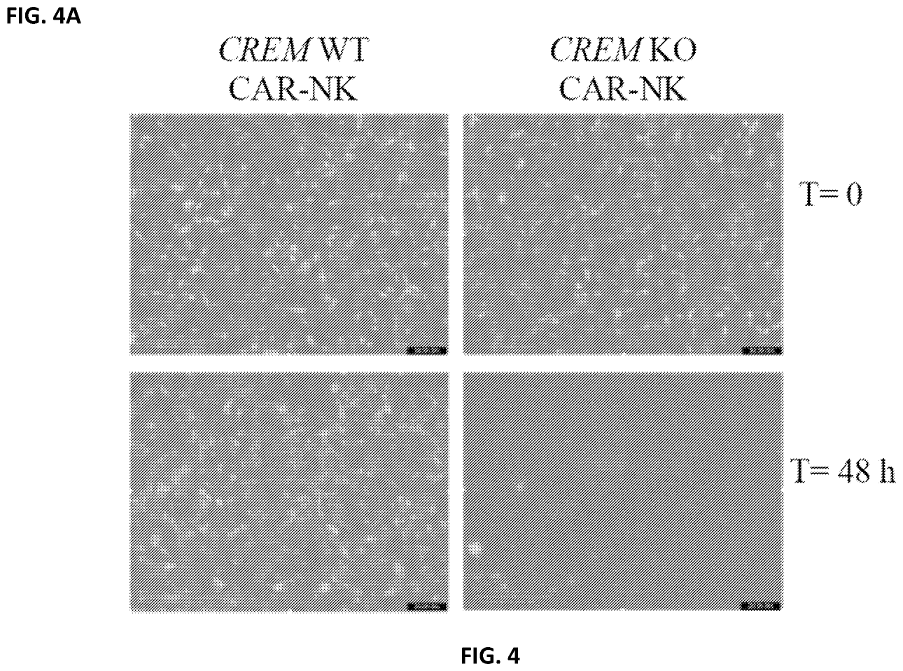

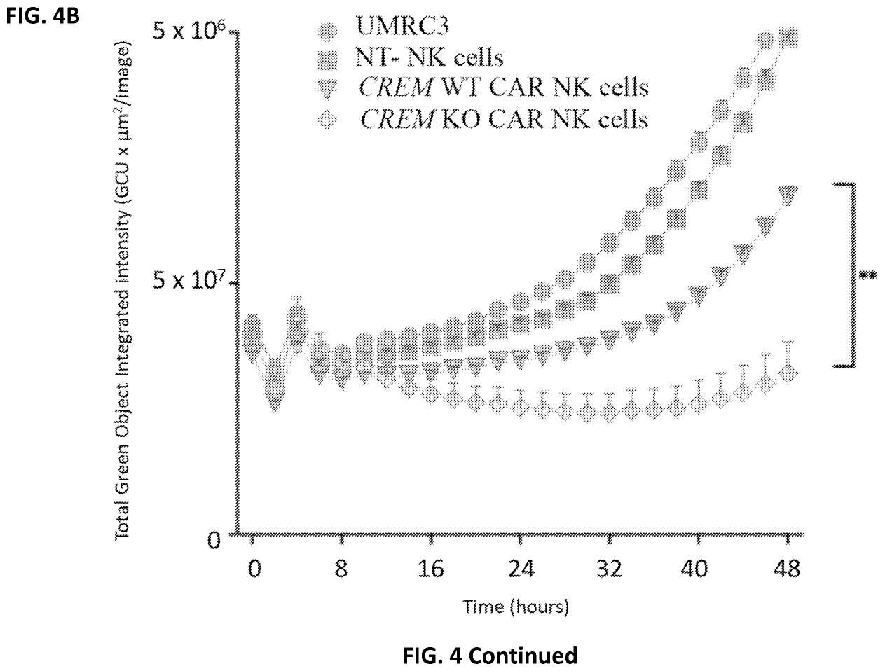

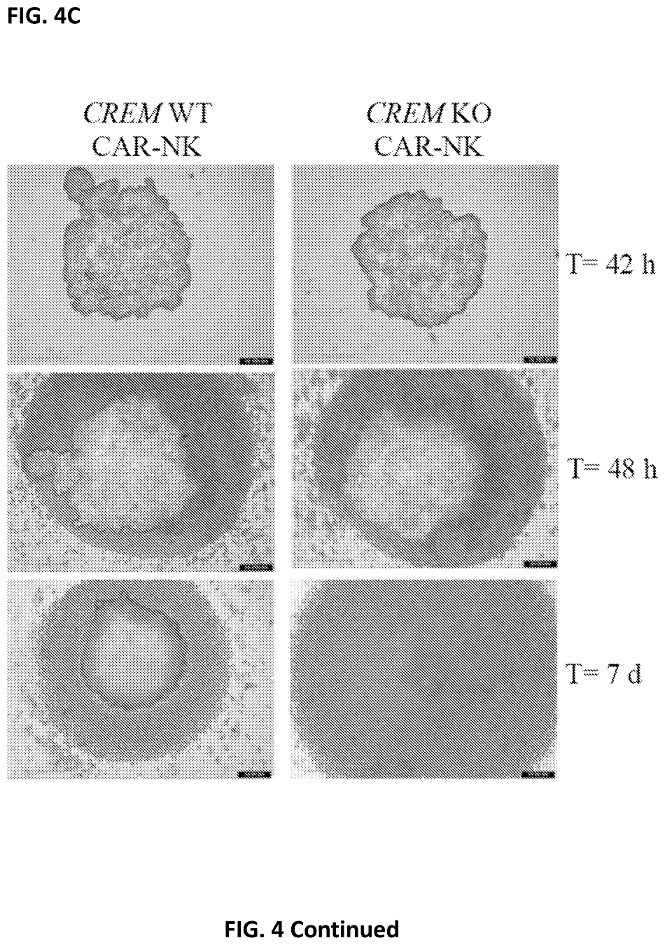

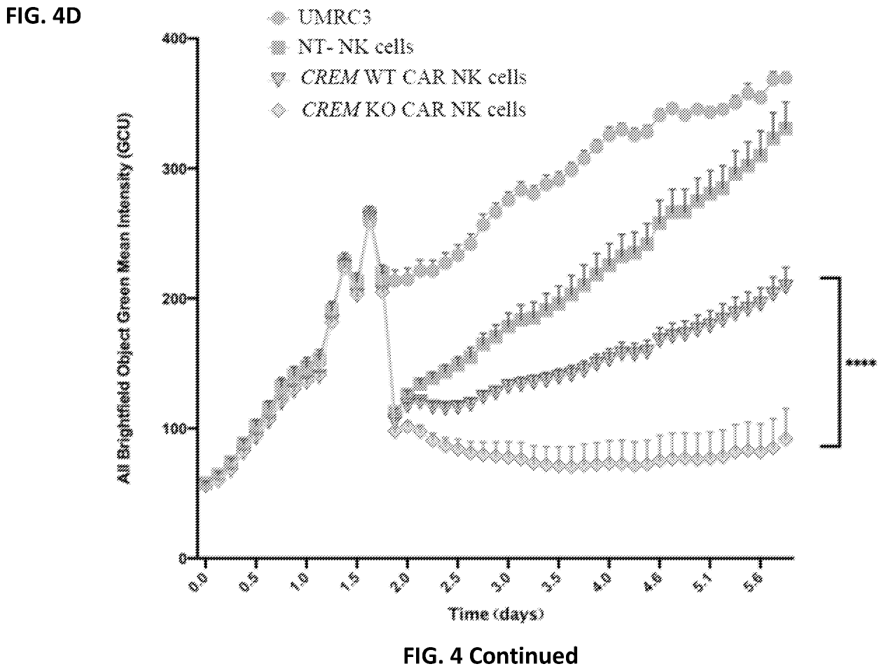

To test the killing potential of CREM WT versus CREM KO CAR-NK cells, long-term killing assays of NK cells against the glycolytic UMRC3 renal cell carcinoma cell line were performed using the INCUCYTE® device with live cell imaging of tumor cell growth and killing by NK cells. The results showed that the activity of CAR-NK cells following CREM KO was enhanced compared to wild-type CAR-NK cells targeting UMRC3 (FIGS. 4A and 4B). To study the effect of CREM KO on the antitumor function of NK cells in conditions that simulate in vivo solid tumor models, killing assays of NK cells against 3D tumor spheroid culture models of UMRC3 cell line were performed. Tumor spheroids mimic solid tumor masses and have previously been shown in the literature to have an acidic pH (see e.g., Nunes et al., 2019). To perform these assays, GFP+UMRC3 cells were seeded in ultra-low attachment plates and 3D tumor spheroids were allowed to form in the INCUCYTER machine for 48 hours, after which NK cells were added. Tumor growth and cell death were monitored in real time. The data showed that CREM KO CAR-NK cells had enhanced cytotoxicity against 3D tumor spheroids when compared to CREM WT CAR-NK cells targeting UMRC3 (FIGS. 4C and 4D).

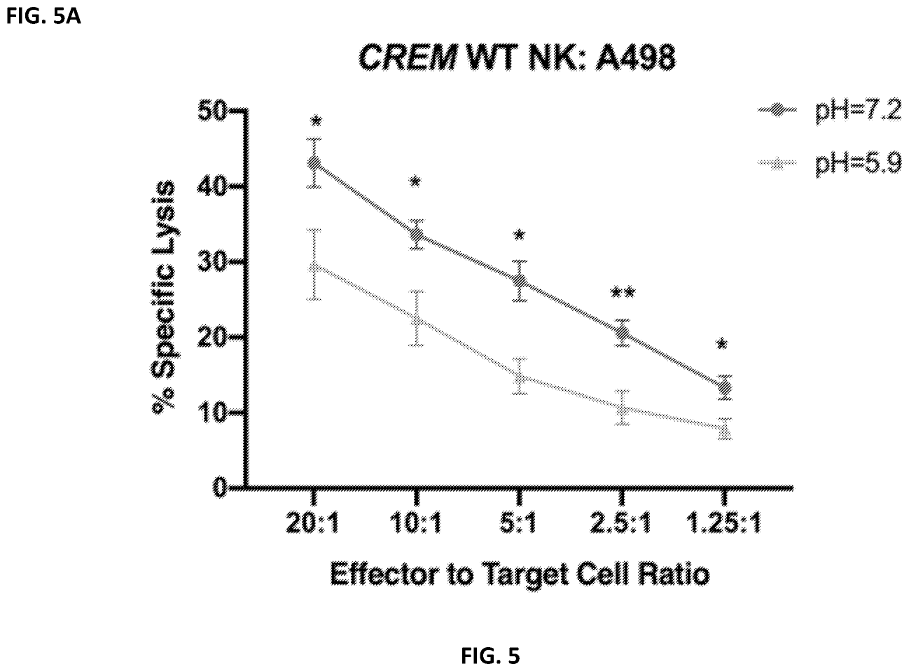

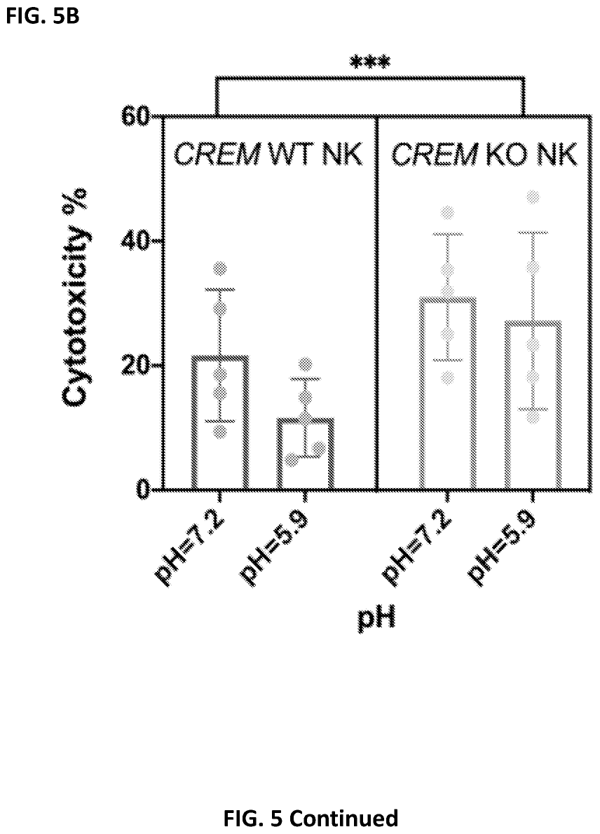

To test the specific effect of CREM KO on abrogating acidity-mediated immunosuppression, chromium release killing assays at various effector: target (E:T) ratios of CREM WT vs. CREM KO NK cells against A498 cell line were performed at 2 pH conditions, a typical culture medium pH (pH-7.2) and an acidic pH (pH=5.9). For CREM WT NK cells, the data showed an expected dampening of NK cell cytotoxicity at an acidic pH (FIG. 5A) but that cytotoxicity was recovered when killing assays were performed using CREM KO NK cells (FIG. 5B).

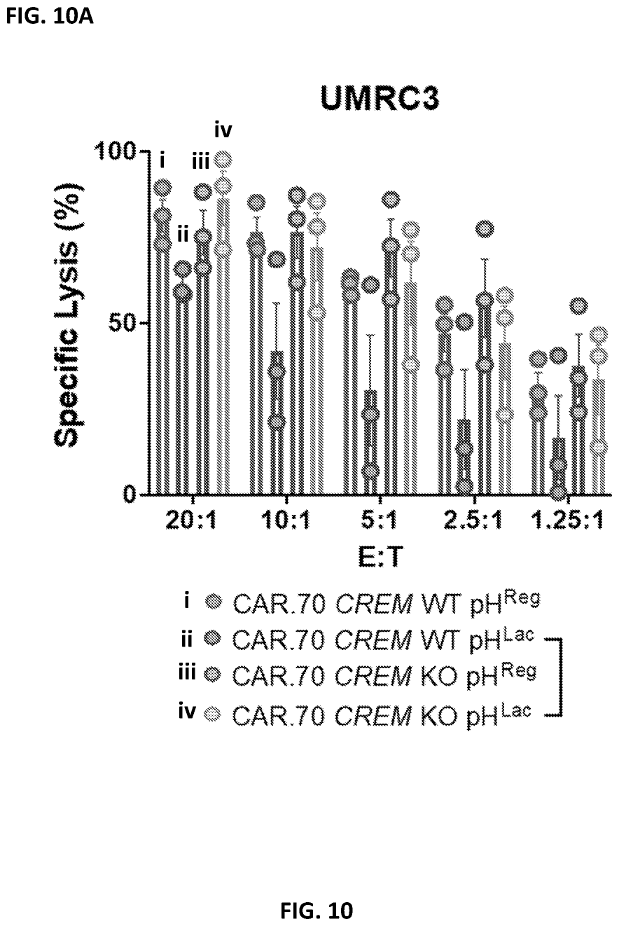

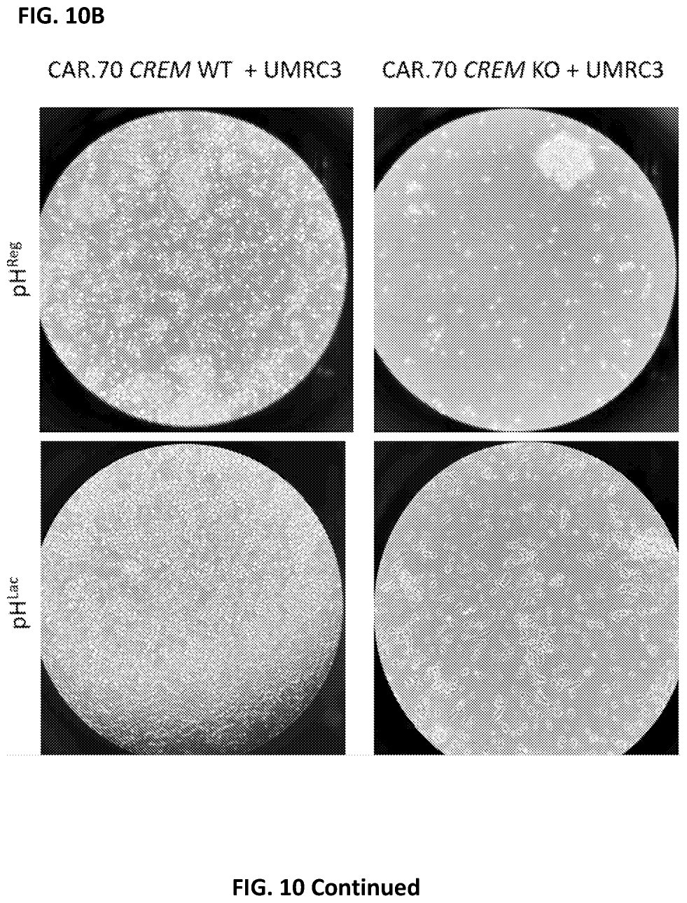

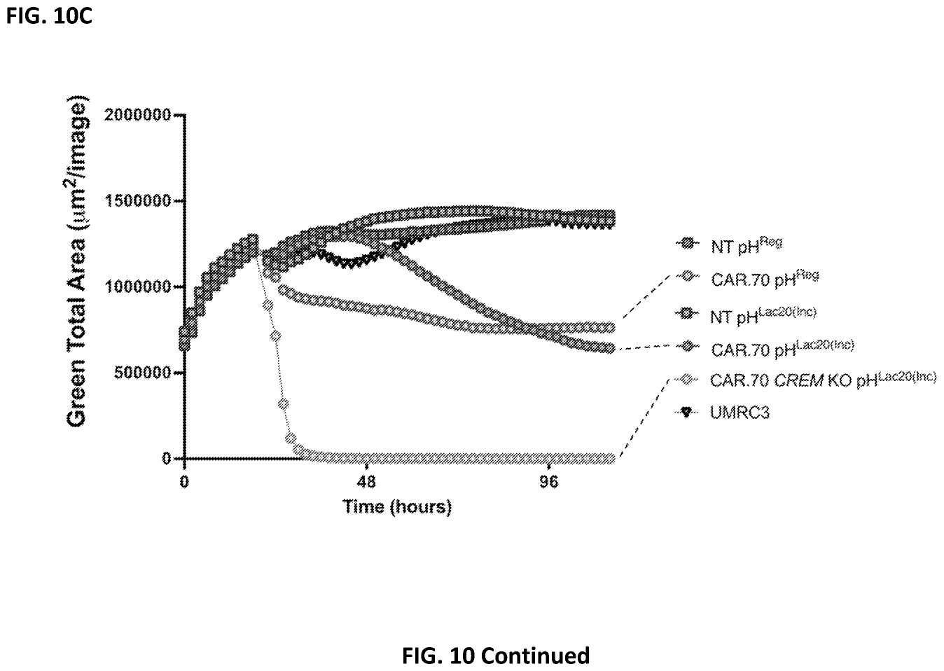

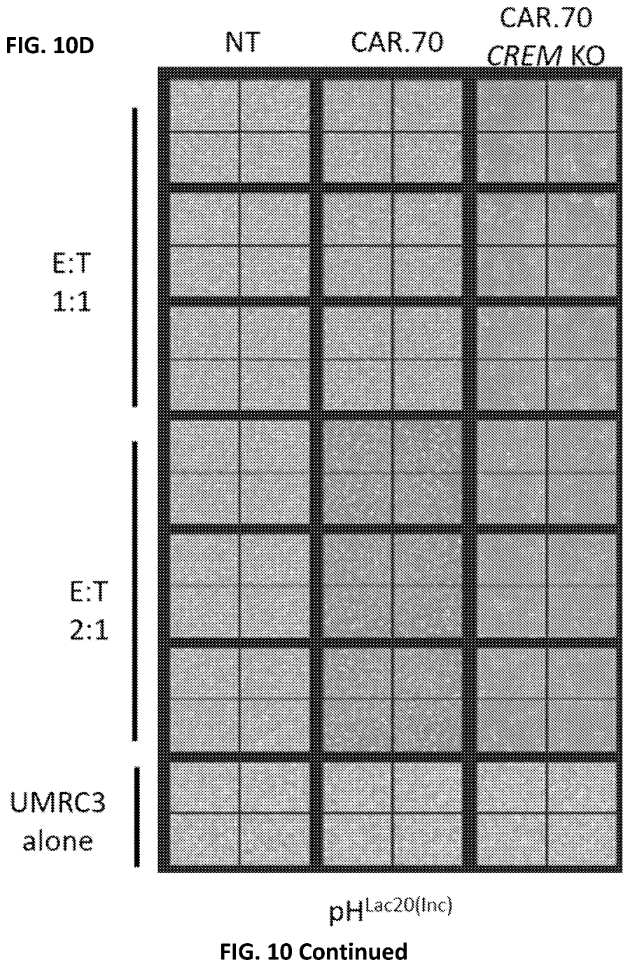

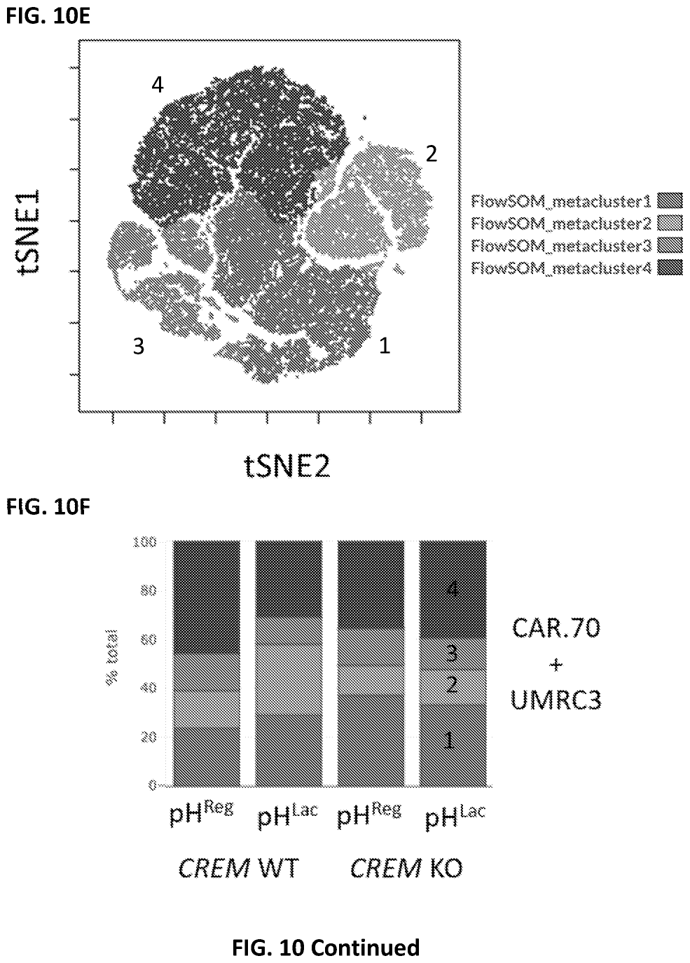

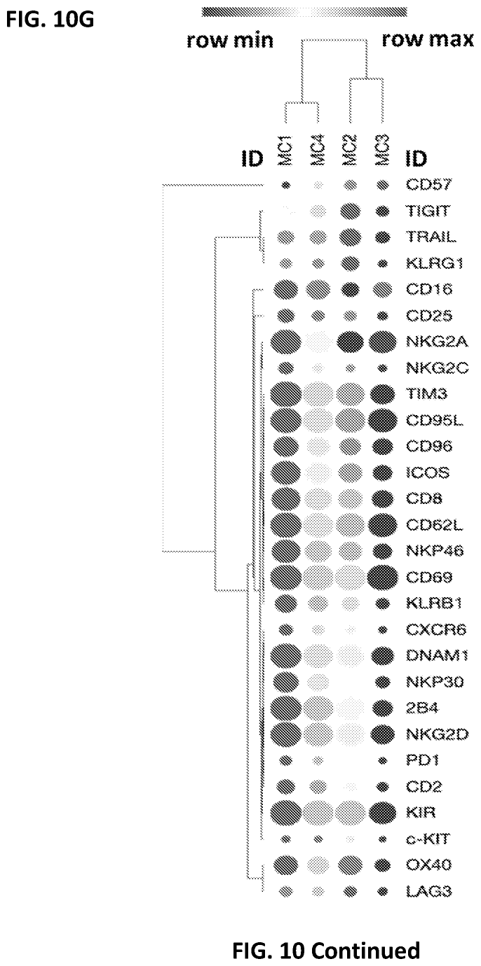

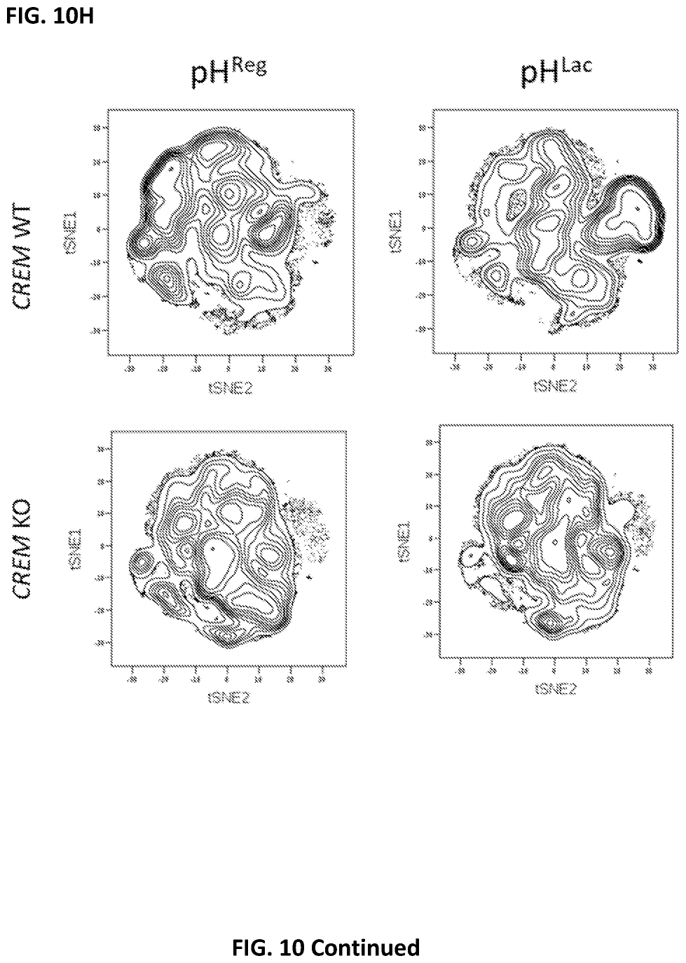

As shown in FIGS. 10A-H, CREM KO enabled CAR-NK cells to withstand the suppressive effects of lactic acid at concentrations similar to those observed in tumor microenvironments (TMEs). As shown in FIG. 10A, the results of 51Cr release assays showed that, in an acidic environment, the cytotoxicity of anti-CD70 CAR-NK (CAR.70) cells against renal cell carcinoma (e.g., UMRC3 renal cell carcinoma tumor cells) at various E:T ratios (e.g., 20:1, 10:1, 5:1, 2.5: and 1.25:1 respectively) was enhanced in CREM KO CAR-NK cells relative to those of CREM WT CAR-NK cells, and comparable to those of CREM WT CAR-NK cells at more neutral pH levels. NK cells were either CREM wild-type (WT) or CREM knock-out (KO), and were incubated in either a regular pH (pH^{Reg}) or an acidic pH (pH^{Lac}) created using lactic acid. Representative brightfield microscopy pictures of CAR-NK cells and UMRC3 tumor cells (elongated cells) cocultured at a regular pH (pH^{Reg}) or an acidic pH (pH^{Lac}) are shown in FIG. 10B. The cell clumps indicated areas of active engagement of cytotoxic CAR-NK cells with the tumor cells. The results showed that CREM KO CAR.70 NK cells exhibited greater levels of clearance of UMRC3 cells relative to CREM WT CAR.70 NK cells. The long term cytotoxicity of CREM WT or CREM KO CAR.70 NK cells was then analyzed using long-term cytotoxicity (e.g., Incucyte®) assays of the CAR-NK cells and their non-transduced (NT) counterparts in pH^{Reg} or pH^{Lac} environments when challenged against UMRC3 cells (FIG. 10C). Acidic conditions (pH^{Lac20(Inc)}) were achieved over 2 weeks by incremental (Inc) increases in cumulative lactic acid concentration (e.g., NK cells were conditioned to incrementally (Inc) more acidic conditions by adding approximately 2.5 mM of lactic acid every 2-3 days, over a 2-week incubation period; pH^{Lac20(Inc)} resulted in a pH of approximately 6.0; all conditions started from the regular pH of culture media (pH^{Reg} ˜7.4)) . . . The results indicated that while chronic lactic acid exposure dampened the cytotoxicity of CAR-NK cells when compared to those in a regular pH, the CREM KO CAR-NK cells were capable of maintaining robust anti-tumor killing despite this relatively extreme condition that mimicked the low pH of the tumor microenvironment of glycolytic tumors. Shown in FIG. 10D are representative Incucyte® images of NT NK cells (NT), CAR.70 CREM WT NK cells (CAR.70), or CAR.70 CREM KO NK cells challenged with GFP+UMRC3 cells at 1:1 or 2:1 E:T ratios in acidic (pH^{Lac20(Inc)}) conditions.

Following confirmation of the enhanced cytotoxicity of CREM KO CAR.70 NK cells relative to CREM WT CAR. 70 NK cells when analyzed at acidic pHs, phenotypic analysis of the NK cells was performed. As shown in FIG. 10E-10H, NK cells (e.g., CREM WT or CREM KO) cocultured with UMRC3 tumor cells under either pH^{Reg} or pH^{Lac} conditions were characterized using multiparametric spectral flow cytometry. viSNE analysis was performed on the various conditions followed by FlowSOM clustering. Shown in FIG. 10E is a colored FlowSOM clustering plot showing metaclusters 1˜4 for the composite of all 4 test conditions. FIG. 10F shows the distribution of the 4 FlowSOM metaclusters (MCs) across the different test conditions. The results demonstrated an increase in MC2 (orange) in CREM WT CAR.70 NK cells cocultured with UMRC3 cells at pH^{Lac}. However, the CREM KO CAR.70 NK cells cocultured with UMRC3 cells in pH^{Lac} exhibited a similar composition of MC2 phenotyped cells as those NK cells cocultured in pH^{Reg} conditions. The underlying phenotypes associated with the metaclusters was then analyzed, FIG. 10G displays expression of various markers in the 4 MCs normalized locally across conditions to minimum and maximum expression on a color scale of blue (minimum) to red (maximum). The percentage expression was overlayed by circles of various sizes proportional to the % expression. MC2 was generally characterized by a relatively low expression of activation markers and a relatively high expression of certain checkpoints (e.g., TIGIT and LAG3) and markers of terminal differentiation (e.g., CD57). The underlying contour tSNE plots of the various test conditions (pH^{Reg} or pH^{Lac}, and CREM WT or CREM KO) was shown in FIG. 10H, where gross phenotypic clustering differences in NK cell distribution following viSNE analysis can be observed.

### Example 4-Crem Ko Enhanced Nk Cell Activation and Polyfunctionality

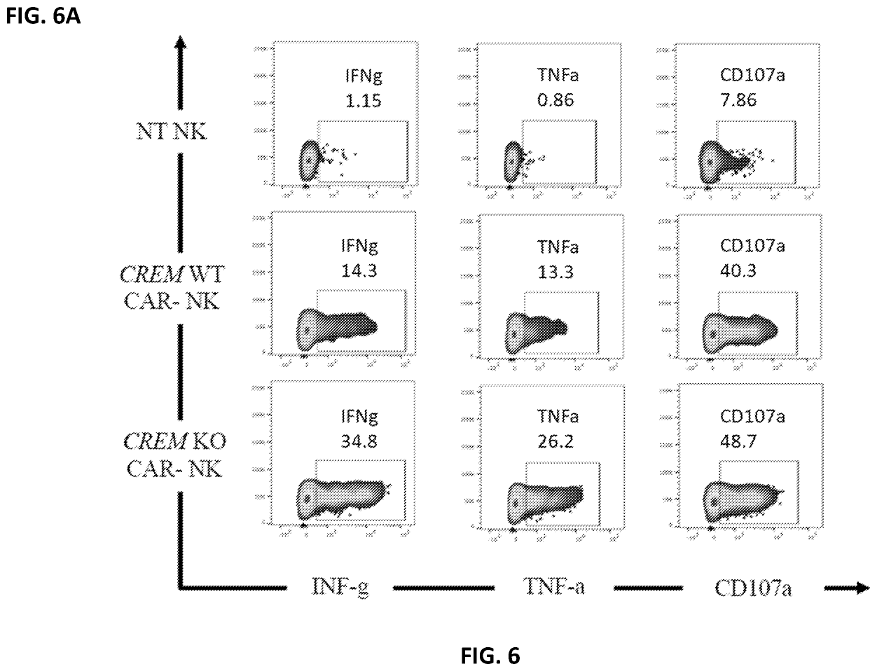

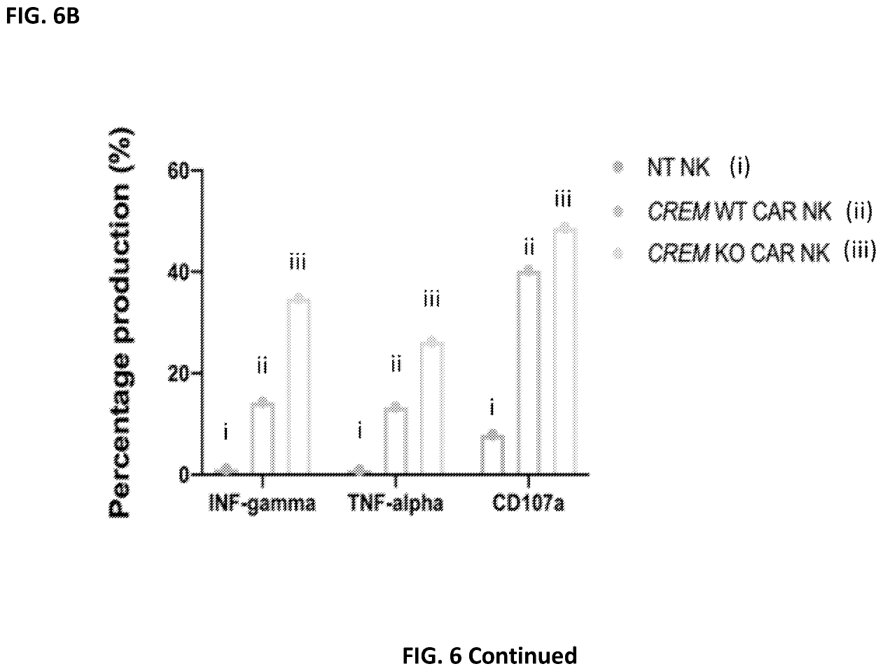

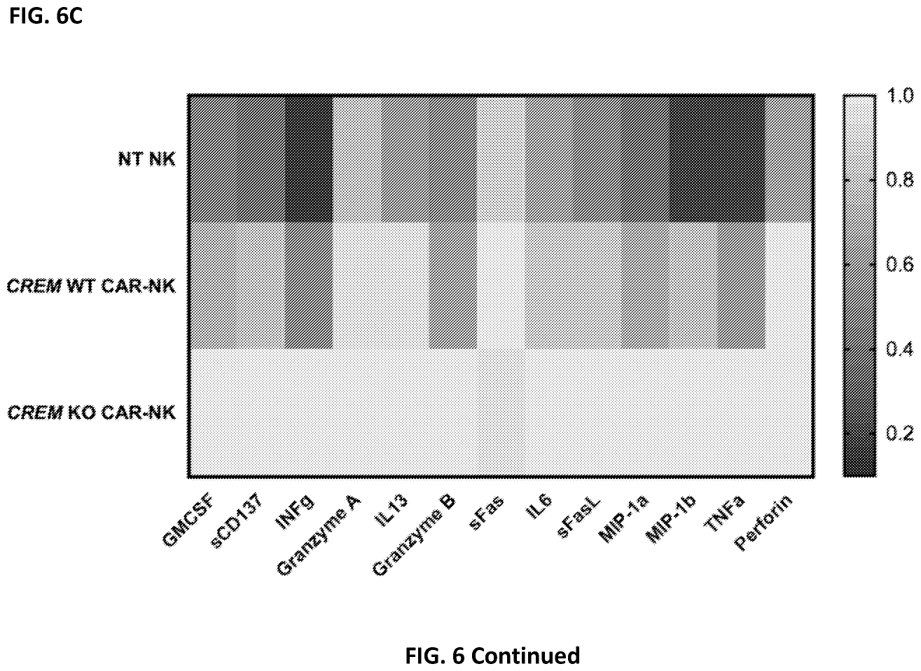

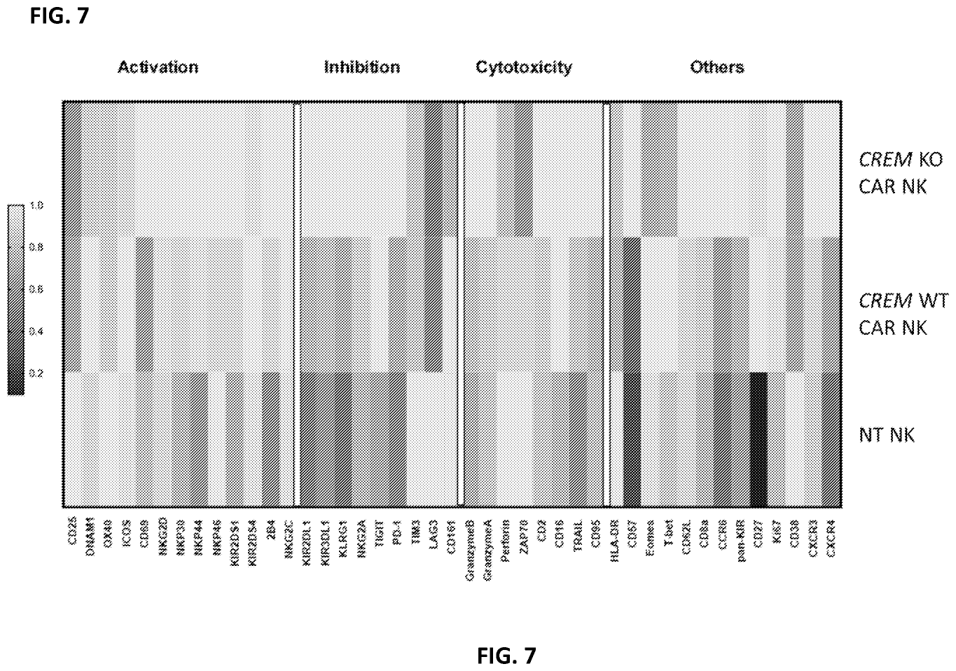

To further elucidate the effect of CREM KO on the functionality of NK cells, coculture assays of NK cells with the glycolytic ovarian cancer cell line SKOV3 were performed, and expression of cytokines in NK cells, such as INF-gamma and TNF-alpha as well as the degranulation marker CD107a were assayed. The results showed that CREM KO further enhanced the capability of CAR-NK cells targeting SKOV3 to produce effective cytokines and express markers of NK cell activation (FIGS. 6A and 6B). Furthermore, in a larger scale polyfunctionality assay, a LUMINEX® assay was utilized to measure a number of cytokines as markers of NK cell activation. Again, the results showed an overall increase in the levels of cytokine section by CREM KO CAR-NK cells when compared to CREM WT CAR-NK cells (FIG. 6C). The various NK cell genotypes were assayed for expression of markers of activation, inhibition, cytotoxicity, and others, through the use of mass cytometry. The data showed that CREM KO CAR-NK cells have an immunophenotype consistent with activated and cytotoxic cells (FIG. 7).

### Example 5-Crem Ko Endowed Nk Cells with Proliferative Advantage without Leading to Autonomous Growth

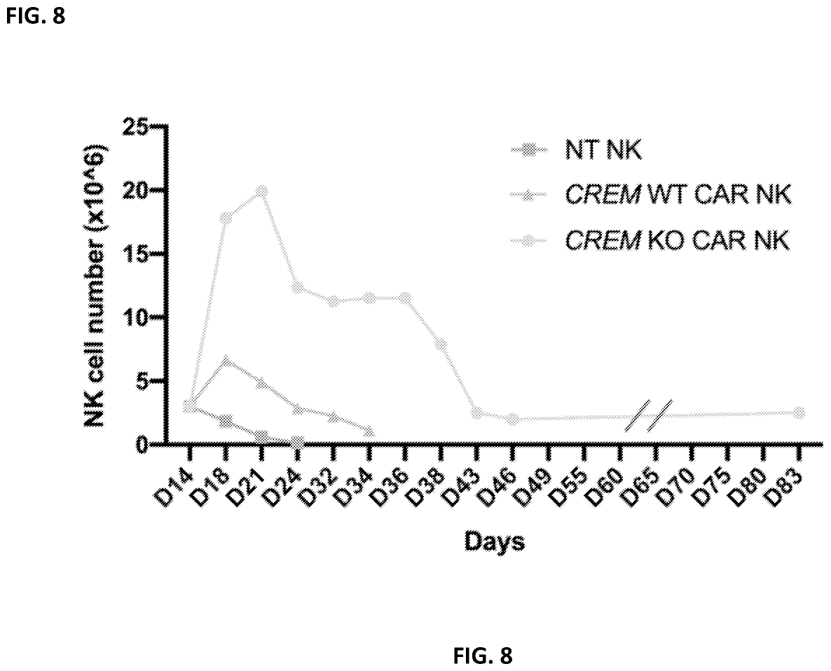

Throughout the aforementioned studies comprising CREM KO NK cells, an increase in proliferation rate of CREM KO NK cells was observed relative their CREM WT counterparts. To assess the effect of CREM KO on NK cell proliferation, while also ensuring that it does not lead to autonomous NK cell growth, the growth of NK cells with or without CREM KO in the absence of IL-2 stimulation (which is typically an essential cytokine for NK cell survival in vitro) was assayed. While enhanced proliferation of CREM WT CAR-NK cells compared to non-transduced (NT) NK cells was observed, which is explained by the fact that the CAR construct also secretes IL-15, the results showed that CREM KO CAR-NK cells had proliferated more and that they persisted longer without additional IL-2. Nevertheless, autonomous growth over time was not observed (FIG. 8).

### Example 6-Crem Ko Endowed Nk Cells with Improved Metabolic Functionality

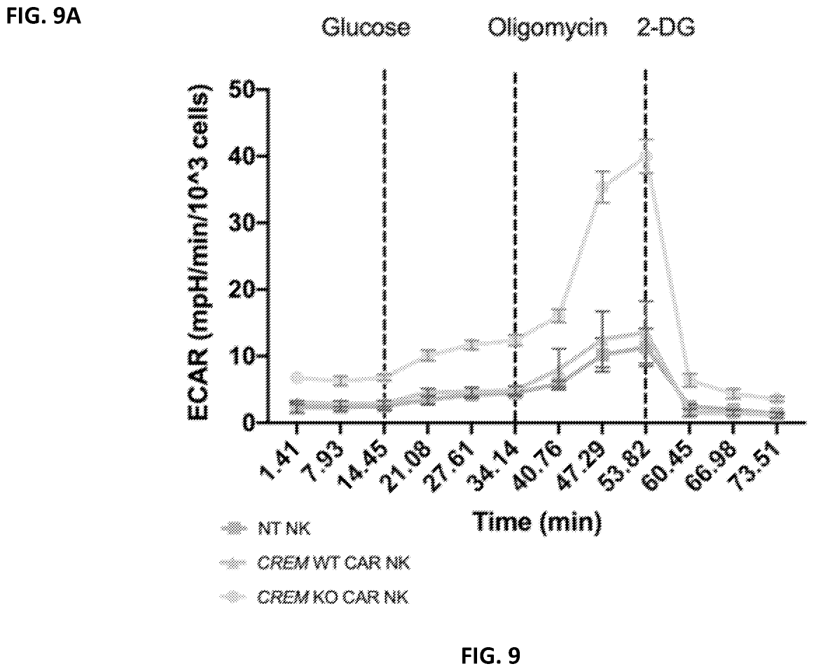

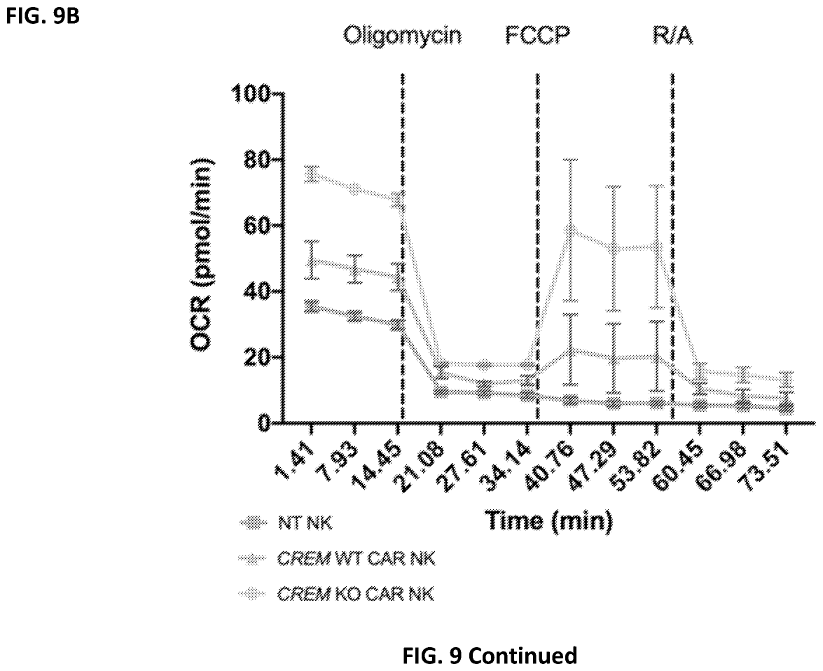

As the aforementioned results had demonstrated an enhanced activity of NK cells in the TME and acidic milieu upon CREM KO, the inventors hypothesized that CREM KO empowers NK cells through metabolic reprogramming that allowed them to compete for metabolites in the TME and to withstand immunosuppression generated by a highly metabolically-active tumor. To test this hypothesis, the glycolytic capacity of CREM KO cells was assayed through measurement of the extracellular acidification rate (ECAR) and oxidative phosphorylation rate through the oxygen consumption rate (OCR). The results showed that CREM KO CAR-NK cells have a higher glycolytic capacity (FIG. 9A) and a better OCR (FIG. 9B) compared to their CREM WT CAR-NK cell counterparts. These results suggested that CREM KO could be rewiring the metabolic program of NK cells and rendering them more fit to resist the negative effects imposed by acidity in the TME.

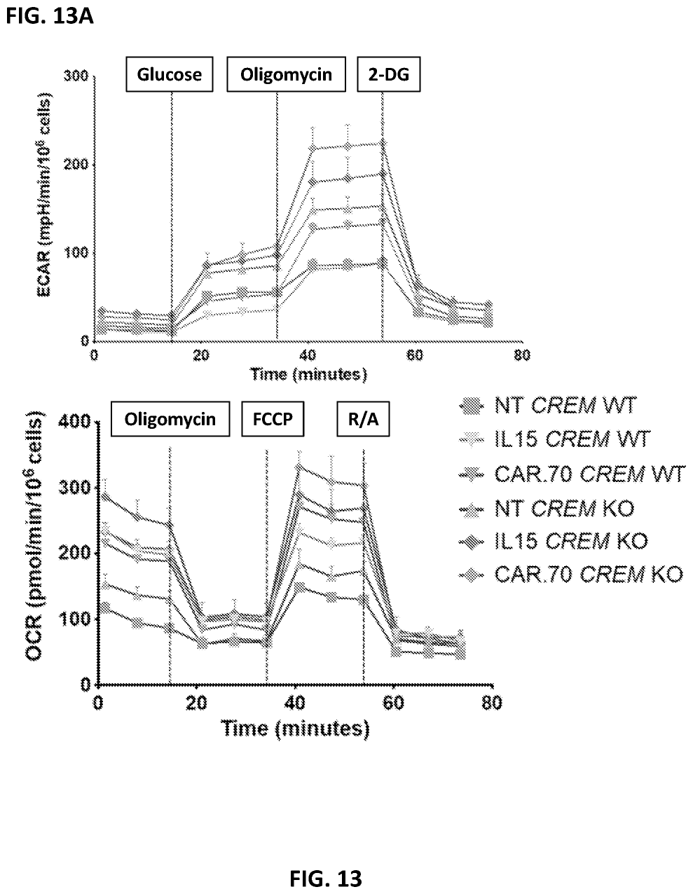

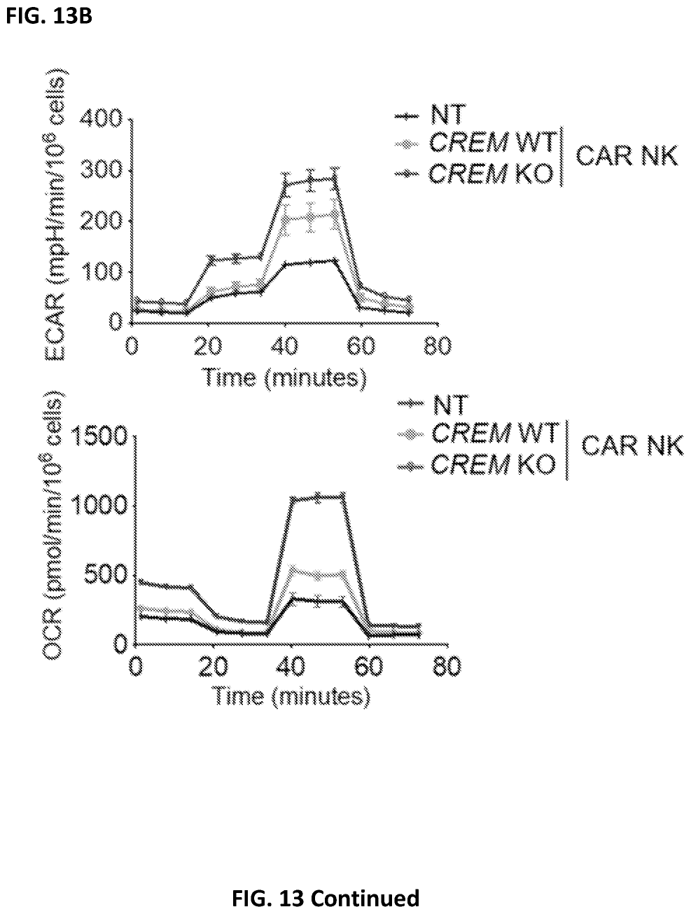

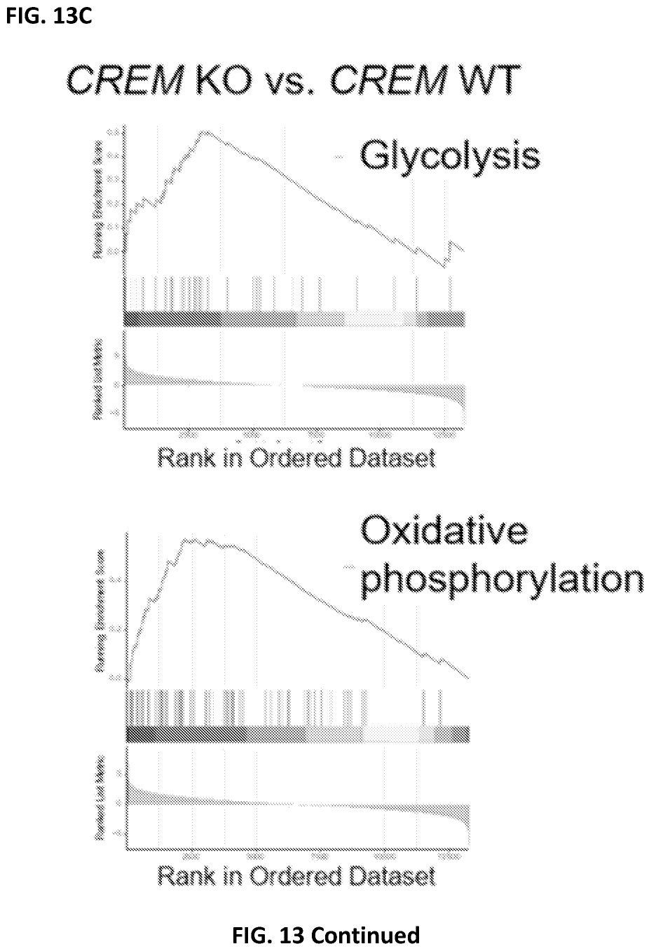

The above results were confirmed as shown in FIGS. 13A-C, where CREM KO was found to enhance the metabolic fitness of NK cells at baseline as well as when cultured in an acidic pH. FIG. 13A depicts the extracellular acidification rate (ECAR; as a surrogate of glycolysis, top panel) of CREM WT or CREM KO anti-CD70 CAR-NK cells (CAR.70) as well as their non-transduced (NT) counterparts. The CAR.70 construct was designed to also secrete interleukin-15 (IL15), and control NK cells that were transduced with a construct expressing IL15 in the absence of a CAR were included in the assay (IL15 NK cells). The assay was performed on NK cells cultured in regular media without stimulation using the Seahorse® glyco stress test assay following the manufacturer's protocol. Oxygen consumption rate (OCR; as a surrogate for oxidative phosphorylation (OXPHOS), bottom panel) was analyzed using the Seahorse® mito stress test assay following the manufacturer's protocol. Provided in FIG. 13B are the results of ECAR (top) and OCR (bottom) of NT, CREM WT CAR-NK cells and CREM KO CAR-NK cells when incubated in lactic acid. Finally, FIG. 13C shows how pathways of glycolysis and OXPHOS were both found to be significantly upregulated in CREM KO NK cells when compared to CREM WT NK cells following incubation with lactic acid (analyzed using unbiased GSEA performed on bulk RNA sequencing of these conditions).

Together these results confirmed that CREM KO could be rewiring the metabolic program of NK cells, and rendering them more fit to resist the negative effects imposed by acidity in the TME.

### Example 7-Crem Ko Improved Car-Nk Cell Activation and Cytotoxicity Ex-Vivo and/or In-Vivo

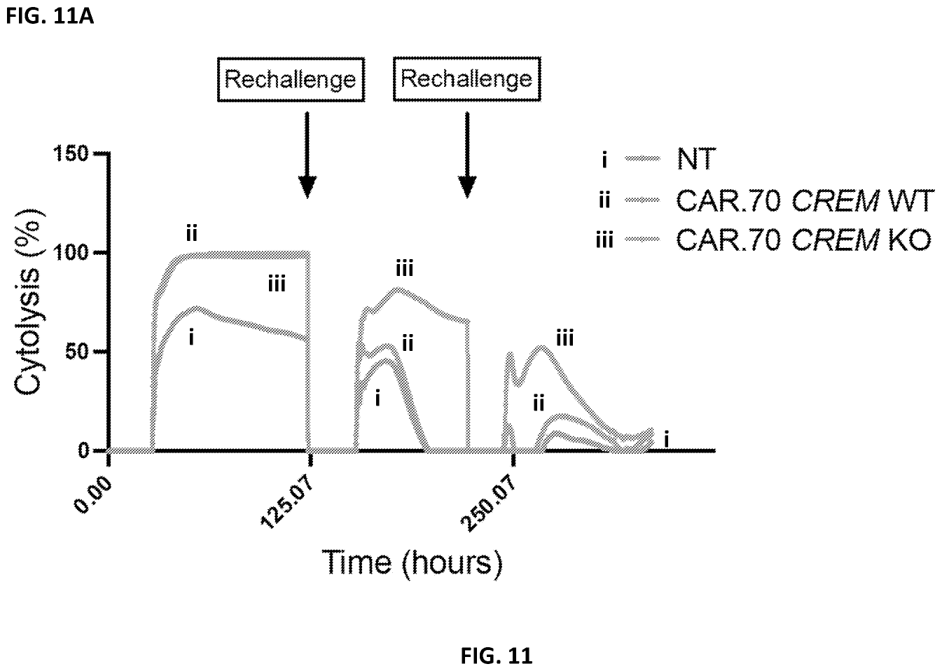

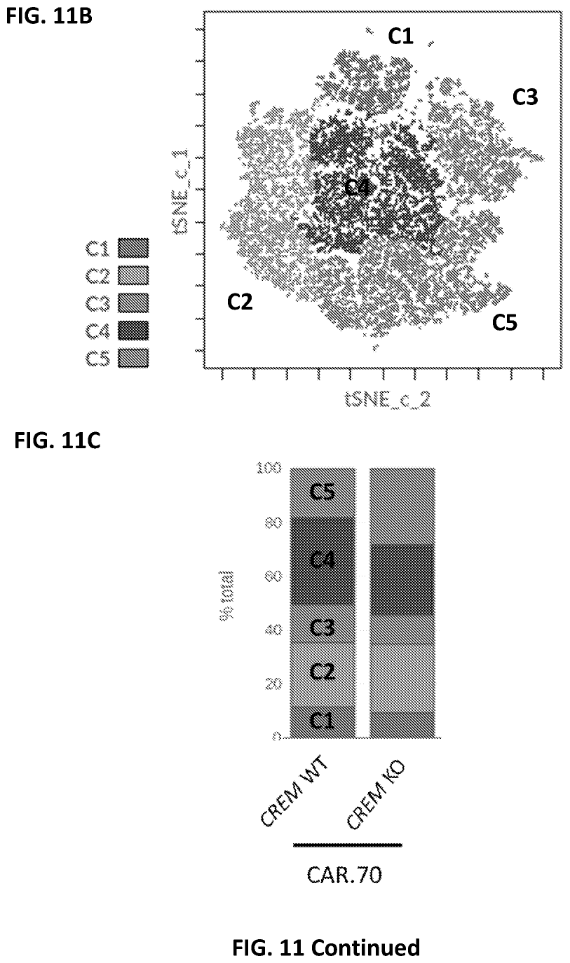

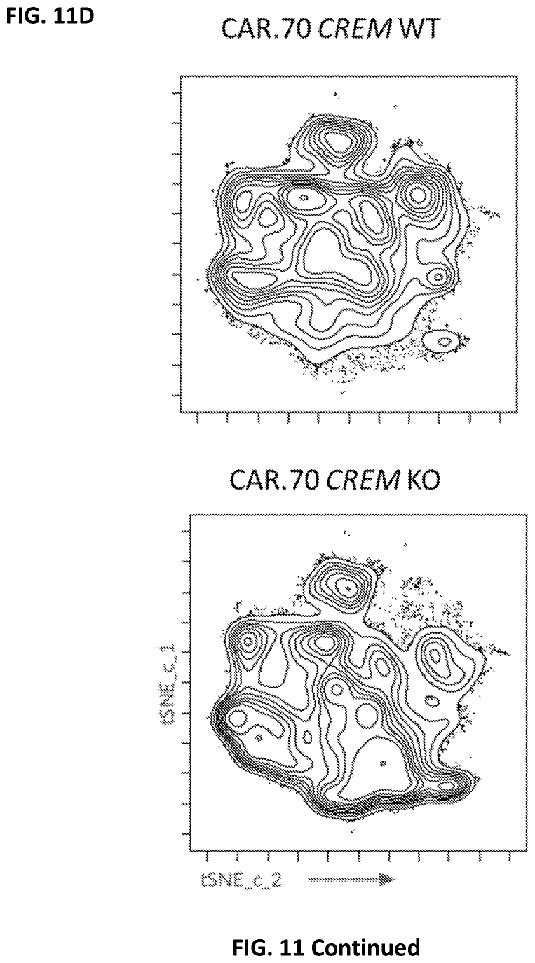

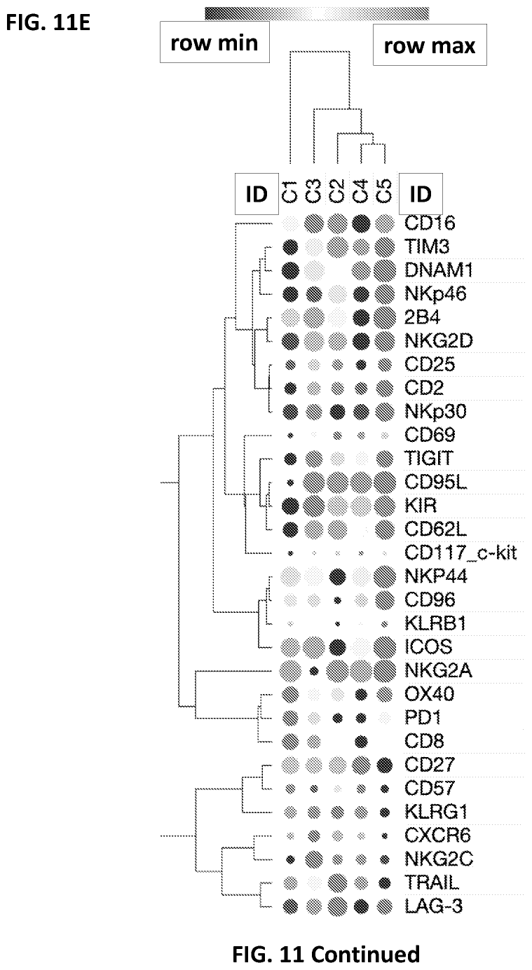

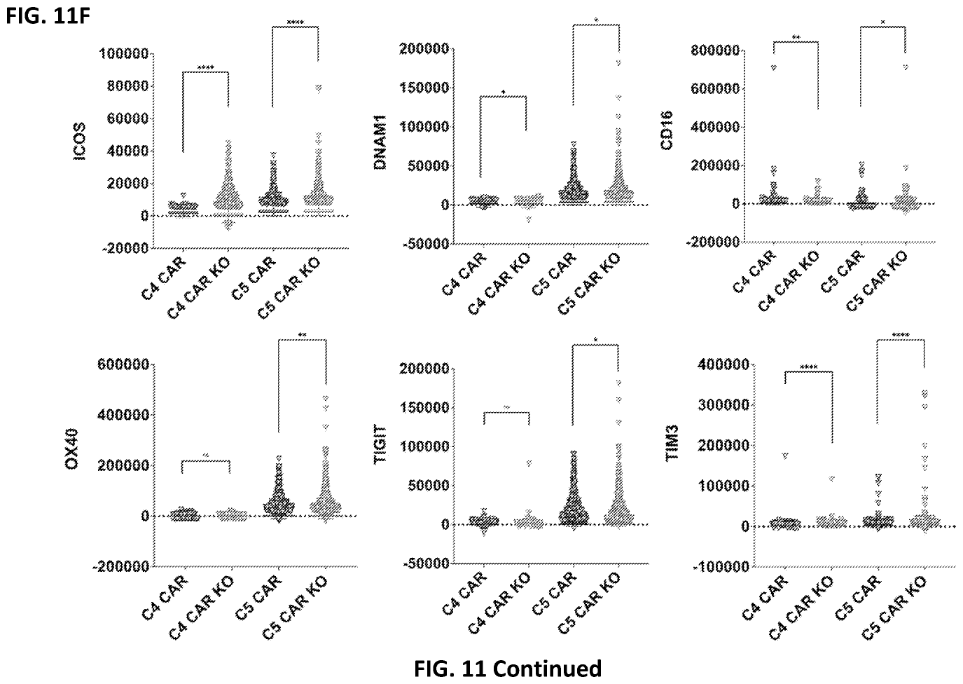

As shown in FIGS. 11A-F, the inventors confirmed CREM KO improved CAR-NK cell activation phenotypes and cytotoxicity, including in long-term assays comprising multiple tumor rechallenges. FIG. 11A displays results of a representative rechallenge cytotoxicity assay using xCELLigence® of CREM WT and CREM KO anti-CD70 CAR-NK cells (CAR.70) and their non-transduced (NT) counterparts against UMRC3 tumor cells. The UMRC3 cells were plated in E-plates and allowed to adhere and grow overnight. Twenty-four hours later, the noted NK cells were added at an E:T ratio of 2:1. Every 2-4 days, new tumor cells were plated in new E-plates. Twenty-four hours later, the NK cells in the first E-plate were collected and counted and transferred at the same E:T ratio of 2:1 onto the new tumor plate. The results showed robust cytolysis of UMRC3 cells by CAR.70 NK cells upon first challenge, and furthermore that CREM KO CAR.70 NK cells displayed increased cytolysis of UMRC3 cells at the first and second tumor cell rechallenges relative to CREM WT CAR.70 NK cells. The CREM WT and CREM KO CAR.70 NK cells were then phenotypically characterized at the end of the rechallenge assay shown in (11A). viSNE analysis was performed on the various conditions followed by FlowSOM clustering into 5 FlowSOM groups (C1-C5) as shown in FIG. 11B. The distribution of the 5 FlowSOM metaclusters (MCs, C1-C5) for cells described in FIGS. 11A-11B were then analyzed and the results were presented in FIG. 11C. The results demonstrated an increase in MC5 and a decrease in MC4 in CREM KO CAR.70 NK cells when compared to CREM WT cells. The underlying contour tSNE plots of CREM WT CAR. 70 NK cells (top) and CREM KO CAR.70 NK cells (bottom) are provided in FIG. 11D, and showed the gross phenotypic clustering differences in their distribution following viSNE analysis. Relative abundance of certain underlying cell markers contributing to the observed phenotypic clustering were identified and provided in FIG. 11E, which shows expression of the various markers in the 5 MCs (C1-C5) normalized locally across conditions to minimum and maximum expression on a color scale of blue (minimum) to orange (maximum). The percentage expression was overlayed by circles of various sizes proportional to the % expression. The results showed that MC4 (which was enriched in CREM WT cells relative to CREM KO cells) was generally characterized by a relatively low level of expression of activation markers, while MC5 (which was enriched in CREM KO cells relative to CREM WT cells) was characterized by an opposite pattern of overall increases in markers of activation. Quantification of select markers of interest at the single cell level was then performed. FIG. 11F shows expression of select markers e.g., ICOS, DNAM1, CD16, OX40, TIGIT, and TIM3, at single cell levels when compared between the CREM WT CAR. 70 NK cells and CREM KO CAR.70 NK cells in MC4 and MC5. The results showed significant differences in marker expression in CREM KO CAR.70 NK cells relative to control CREM WT CAR.70 NK cells. These results confirmed that when cultured with target tumor cells, CREM KO NK cells displayed different phenotypic profiles representative of greater levels of activation when compared to CREM WT NK cells.

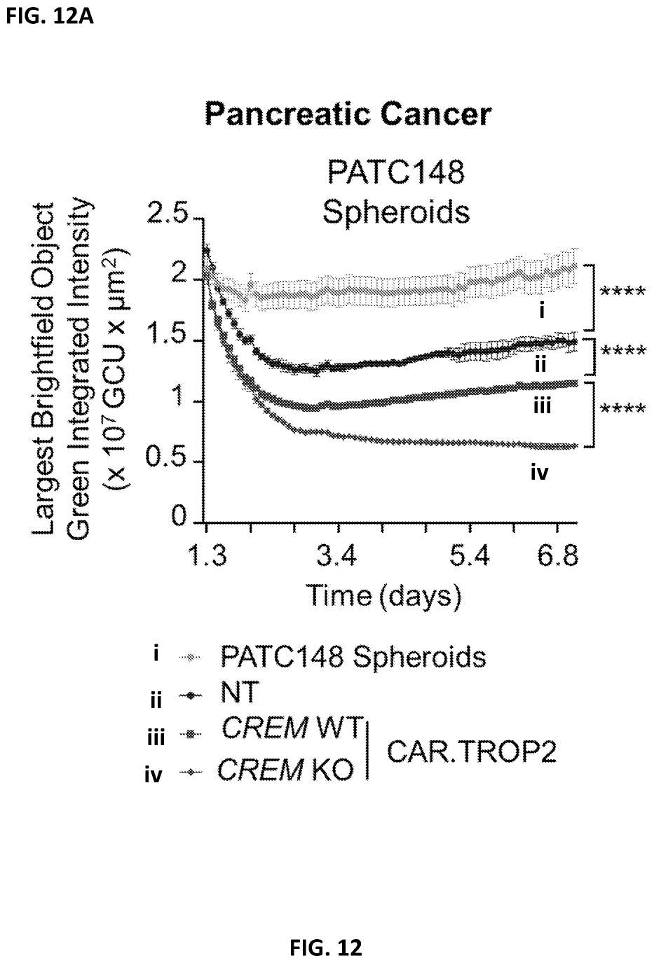

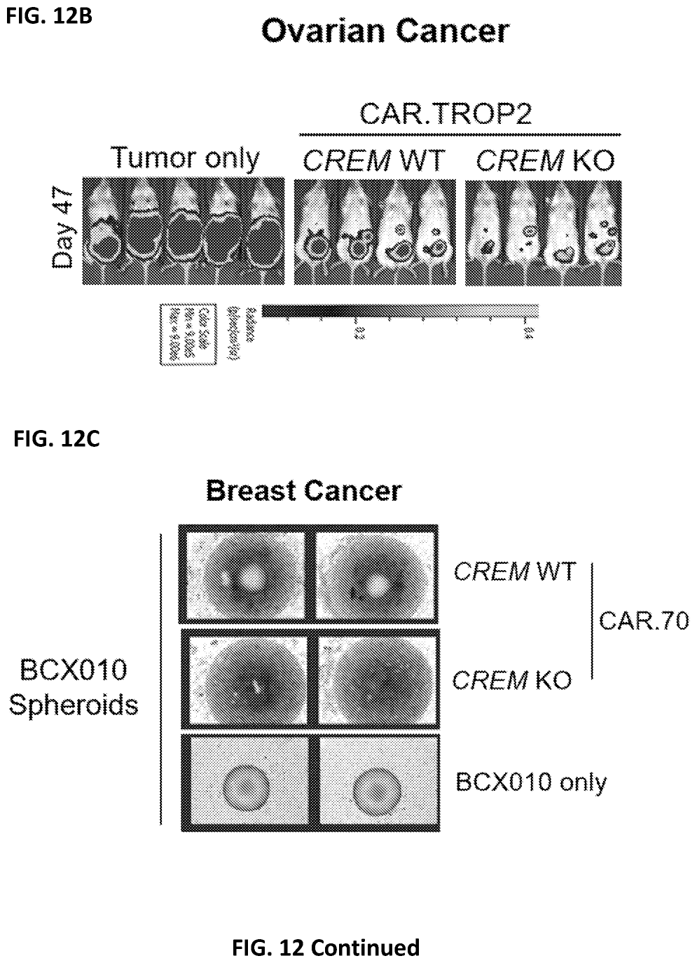

As shown in FIG. 12A-C, the inventors then confirmed the enhanced cytotoxicity of CREM KO CAR-NK cells in multiple different tumor models, using multiple different CAR constructs, while targeting multiple different tumor antigens, in both in vitro 3D tumor spheroid models and in vivo mouse models. FIG. 12A shows how CREM KO significantly enhanced cytotoxicity of TROP2-directed CAR-NK cells (CAR.TROP2) against 3D spheroids of pancreatic cancer cells (e.g., GFP+PATC148 cells) relative to CREM WT TROP-2 directed CAR-NK cells. Shown in FIG. 12B are representative bioluminescence imaging (BLI) of mouse groups injected peritoneally with TROP2+expressing ovarian cancer cells (luciferase positive SKOV3 cells) only, injected with tumor cells and CREM WT CAR.TROP2 CAR-NK cells, or injected with tumor cells and CREM KO CAR.TROP2 CAR-NK cells. Six days following tumor implantation, the mice were irradiated, the following day mice were treated intraperitoneally with 4 million NK cells (CAR.TROP2 transduced NK cells that were CREM WT or CREM KO respectively). The provided representative images showed the burden of luciferase positive tumor cells on day 47 after NK cell injection, where CAR.TROP2 NK cells showed reduced tumor burden relative to tumor only animals, and where CREM KO CAR.TROP2 NK cell injected animals showed reduced tumor burden relative to CREM WT CAR.TROP2 NK cell injected animals. These results showed that CREM KO enhanced the in vivo cytotoxicity of CAR NK cells against solid tumors. Provided in FIG. 12C are representative images of cytotoxicity assays of CREM WT or CREM KO anti-CD70 (CAR.70) CAR-NK cells tested against CD70-positive breast cancer spheroids (e.g., breast cancer cell line BCX010) when assayed using Incucyte®. BCX010 (GFP-positive) cells were plated in ultra low-attachment (ULA) plates and allowed to form spheroids over 24 hours. The following day, NK cells were added to the corresponding wells. The results showed CREM KO CAR-NK cells reduced CD70+tumor cell fluorescence relative to CREM WT CAR-NK cells.

Together, the provided results showed that CREM KO improved CAR-NK cell activation and cytotoxicity ex-vivo and/or in-vivo in multiple different cancer models and when directed against various antigens.

### Example 8-Crem Ko Nk Cells Displayed Distinct Transcriptomic And Epigenetic Profiles in Acidic Environments

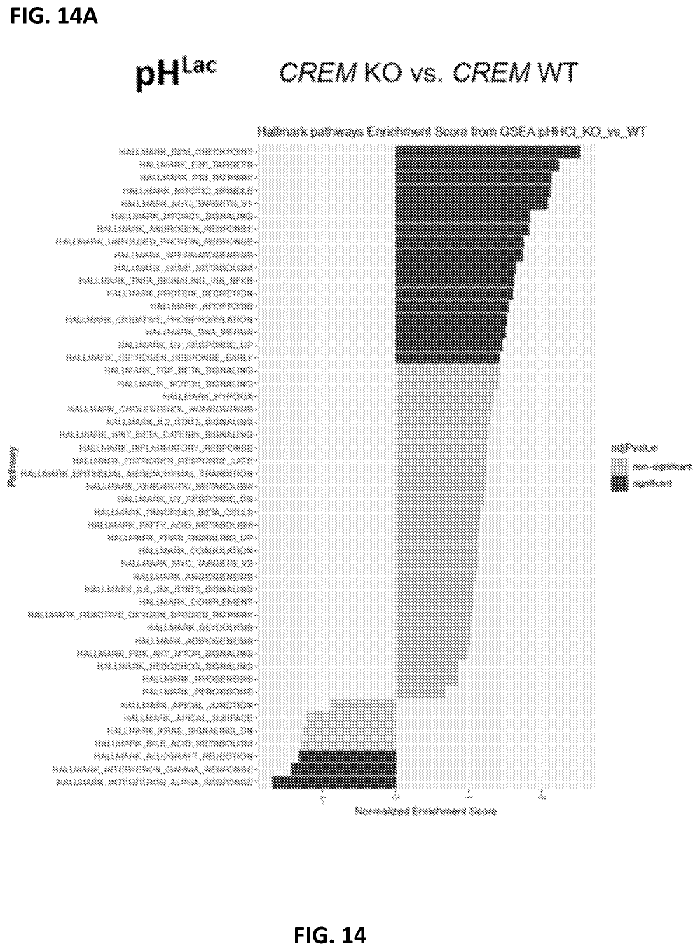

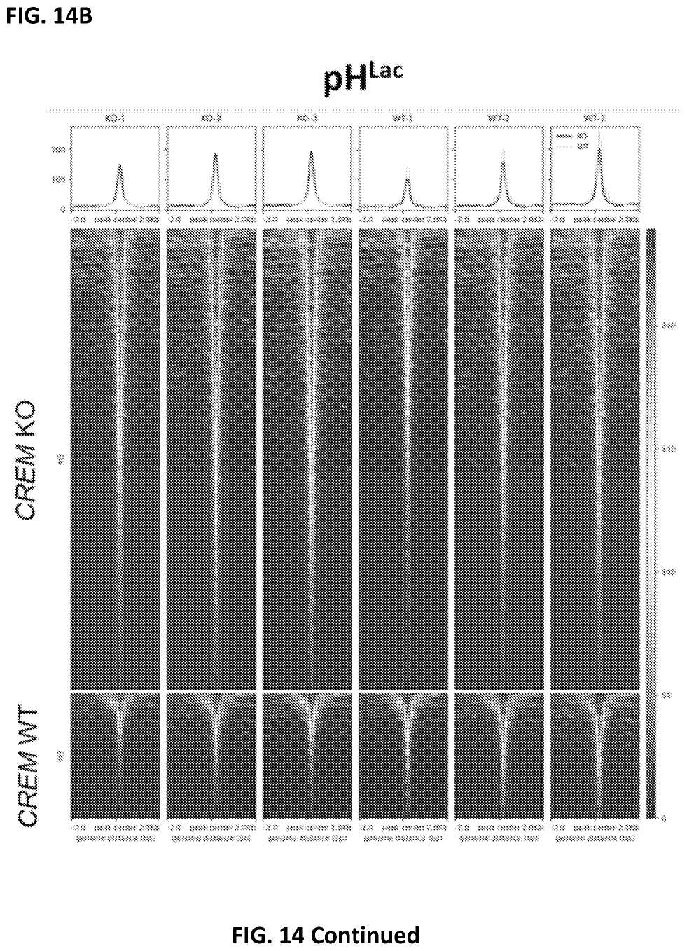

The inventors then analyzed the transcriptomic and epigenetic profiles of CREM KO and CREM WT NK cells when exposed to acidic environments (e.g., microenvironments) comprising relatively high quantities of lactic acid (e.g., conditions mimicking the TME, e.g., pH^{Lac}). The associated results are provided in FIGS. 14A-B, where CREM KO NK cells displayed distinct transcriptomic and epigenetic profiles when compared to CREM WT NK cells upon incubation with lactic acid. FIG. 14A provides a bar graph of differentially upregulated or downregulated HALLMARK pathways in CREM KO vs. CREM WT NK cells incubated in lactic acid for 24 hours. Analysis was performed using gene set enrichment analysis (GSEA) on bulk RNA sequencing of the samples. Notably, CREM KO was associated with upregulation of pathways of proliferation (e.g., G2M, E2F), MYC, MTORC1, oxidative phosphorylation, and TNFa signaling. With the cells displaying distinct transcriptomic profiles, the inventors then determined if the NK cell's epigenetic profiles were altered. As shown in FIG. 14B, chromatin accessibility CREM KO vs. CREM WT NK cells incubated in lactic acid for 24 hours was found to be different (as assessed by bulk ATAC sequencing of the corresponding samples). Each row represented one peak displayed over a 4-kb window. Three cord blood donors of NK cells were used (WT-1, WT-2, WT-3) as well as their CREM KO counterparts (KO-1, KO-2, KO-3). Notably, CREM KO was associated with a pattern of significantly increased chromatic accessibility across the genome.

In summary, the data showed that mutating the CREM gene (e.g., knocking out the CREM gene), that encodes a key transcription factor downstream of proton-sensing GPRs, provided NK cells with improved antitumor activity in acidic conditions as well as in in-vivo-like conditions of 3-D tumor spheroids, a condition that simulates the in-vivo acidic TME conditions, and in in-vivo conditions when tested against solid tumors. These results provided proof-of-concept results for targeting the CAMP pathway to alleviate the immunosuppressive phenotypes induced through the acidity of a TME.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### Patents and Patent Applications

U.S. Pat. No. 4,870,287
U.S. Pat. No. 5,760,395
U.S. Pat. No. 5,844,905
U.S. Pat. No. 5,885,796
U.S. Pat. No. 6,143,866
U.S. Pat. No. 6,207,156
U.S. Pat. No. 6,410,319
U.S. Pat. No. 6,451,995
U.S. Pat. No. 7,070,995
U.S. Pat. No. 7,265,209
U.S. Pat. No. 7,354,762
U.S. Pat. No. 7,446,191
U.S. Pat. No. 7,446,190
U.S. Pat. No. 7,446,179
U.S. Pat. No. 8,017,114
U.S. Pat. No. 8,119,129
U.S. Pat. No. 8,252,592
U.S. Pat. No. 8,324,353
U.S. Pat. No. 8,329,867
U.S. Pat. No. 8,339,645
U.S. Pat. No. 8,398,282
U.S. Pat. No. 8,479,118
U.S. Patent Publication No. US 2005/0260186
U.S. Patent Publication No. US 2006/0104968
U.S. Patent Publication No. US 2002/131960
U.S. Patent Publication No. US 2013/287748
U.S. Patent Publication No. US 2013/0149337
European patent application number EP2537416
PCT Application No. PCT/US19/62009
WO 1995/001994
WO 1998/042752
WO 2000/14257
WO 2000/37504
WO 2001/014424
WO 2013/126726
WO 2012/129514
WO 2013/166321
WO 2013/071154
WO 2013/123061
WO 2014/055668
WO 2014/031687

### PUBLICATIONS

Ausubel et al., Current Protocols in Molecular Biology, Greene Publishing Associates and John Wiley δ Sons, NY, 1994.
Bohn T., et al., Nature Immunology 19 (12): 1319-29, 2018.
Camacho et al. (2004) J Clin Oncology 22 (145): Abstract No. 2505.
Chothia et al., EMBO J. 7:3745, 1988.
Courtney K D, Bezwada D, Mashimo T, et al., Cell Metabolism 28:793-800.e2, 2018.
Davila et al. PLOS ONE 8 (4): e61338, 2013.
Fedorov et al., Sci. Transl. Medicine, 5 (215), 2013.
Huber V, Camisaschi C, Berzi A, et al. Semin Cancer Biol 43:74-89, 2017.
Hurwitz et al. (1998) Proc Natl Acad Sci USA 95 (17): 10067-10071.
Ishii S, Kihara Y and Shimizu T., J Biol Chem 280:9083-7, 2005.
Janeway et al, Immunobiology: The Immune System in Health and Disease, 3rd Ed., Current Biology Publications, p. 433, 1997.
Jores et al., PNAS U.S.A. 87:9138, 1990.
Kabat et al., “Sequences of Proteins of Immunological Interest, US Dept. Health and Human Services, Public Health Service National Institutes of Health, 1991, 5th ed.
Leal, M., Ann N Y Acad Sci 1321, 41-54, 2014.
Lefranc et al., Dev. Comp. Immunol. 27:55, 2003.
Ludwig M-G, Vanek M, Guerini D, et al., Nature 425:93-98, 2003.
Maghazachi A A, Knudsen E, Jin Y, et al., Biochem Biophys Res Commun 320:810-5, 2004.
Mokyr et al. (1998) Cancer Res 58:5301-5304.
Mosenden R., and Taskén K., Cell signaling 23 (6): 1009-16, 2011.
Nunes A S, Barros AS, Costa E C, et al., Biotechnol Bioeng 116:206-226, 2019.
Onozawa Y, Fujita Y, Kuwabara H, et al., Eur J Pharmacol 683:325-31, 2012.
Perez et al., Cell. 1990 Oct. 19;63 (2): 251-8.
Robert R and Mackay CR, Immunol Cell Biol 96:341-343, 2018.
Renner K, Singer K, Koehl G E, et al. Frontiers in Immunology 8:248-248, 2017.
Sadelain et al., Cancer Discov. 3 (4): 388-398, 2013.
Sambrook et al., Molecular Cloning: A Laboratory Manual, 3^{rd} ed., Cold Spring Harbor Press, Cold Spring Harbor, N.Y. 2001.
Singh et al., Cancer Research, 68:2961-2971, 2008.
Singh et al., Cancer Research, 71:3516-3527, 2011.
Sisignano M., Fischer M. J. M., and Geisslinger G., Cells 10 (8): 2050, 2021.
Sun D., et al., Nucleic Acids Research 49 (D1): D1420-D30, 2020.
Turtle et al., Curr. Opin. Immunol., 24 (5): 633-39, 2012.
Wang J Q, Kon J, Mogi C, et al., J Biol Chem 279:45626-33, 2004.
Wen A Y, Sakamoto K M, Miller L S. J Immunol 185:6413-9, 2010.
Wu et al., Cancer, 18 (2): 160-75, 2012.
Verjans E., et al., Oncotarget 6 (36): 38538-51, 2015.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. An engineered immune effector cell, wherein the cell comprises one or more engineered mutations in an endogenous cAMP response element modulator (CREM), G-protein coupled receptor 4 (GPR4), G-protein coupled receptor 31 (GPR31), G-protein coupled receptor 68 (GPR68), G-protein coupled receptor 81 (GPR81), G-protein coupled receptor 132 (GPR132), G-protein coupled receptor 151 (GPR151), inducible cAMP early repressor (ICER), and/or cyclic AMP-responsive element-binding protein 1 (CREB1) gene of the cell.

2. The cell of claim 1, wherein the mutation is a partial or complete loss of function, and/or knock-out (KO) mutation.

3. The cell of claim 1 or 2, wherein the mutation reduces or inhibits transcription or post-transcriptional processing of one or more mRNA isoforms encoded by the mutated endogenous gene relative to a non-mutated locus encoding the same endogenous gene.

4. The cell of claim 1, wherein the mutation is a neomorphic or gain of function mutation.

5. The cell of claim 1 or 4, wherein the mutation increases transcription or post-transcriptional processing of one or more mRNA isoforms encoded by the mutated endogenous gene relative to a non-mutated locus encoding the same endogenous gene.

6. The cell of any one of claims 1-5, wherein the mutation results in a modified mRNA isoform population encoded by the mutated endogenous gene relative to a representative mRNA population encoded by a non-mutated locus encoding the same endogenous gene.

7. The cell of any one of claims 1-4, wherein the mutation results in a modified protein isoform population encoded by the mutated endogenous gene relative to a representative protein population encoded by a non-mutated locus encoding the same endogenous gene.

8. The cell of any one of claims 1-7, wherein the mutation comprises a knock-out (KO) mutation in the endogenous gene of the cell.

9. The cell of any one of claims 1-7, wherein the mutation comprises a homozygous mutation in the endogenous gene of the cell.

10. The cell of any one of claims 1-7, wherein the mutation comprises a heterozygous mutation in the endogenous gene of the cell.

11. The cell of any one of claims 1-10, wherein the mutation results in improved cytotoxicity of the engineered cell in an acidic microenvironment and/or a tumor microenvironment (TME) relative to a control non-engineered cell.

12. The cell of any one of claims 1-11, wherein the mutation results in improved cytotoxicity of the engineered cell in an acidic microenvironment with a pH less than or equal to about 7.0 relative to control non-engineered cell.

13. The cell of any one of claims 1-12, wherein the mutation results in improved cytotoxicity of the engineered cell in an acidic microenvironment with a pH less than or equal to about 5.9 relative to a control non-engineered cell.

14. The cell of any one of claims 1-13, wherein the mutation results in improved cytotoxicity of the engineered cell, optionally in an acidic microenvironment characterized by increased levels of lactic acid relative to a non-acidic microenvironment.

15. The cell of any one of claims 1-14, wherein the mutation results in enhanced polyfunctionality of the engineered cell relative to a control non-engineered cell in response to stimulation by tumor cells.

16. The cell of claim 15, wherein the enhanced polyfunctionality is evidenced by an increase in cytokine release in response to stimulation by tumor cells.

17. The cell of claim 16, wherein the increase in cytokine release comprises an increase in interferon gamma (IFN-g), tumor necrosis factor alpha (TNF-a), and/or the degranulation marker CD107a, in response to stimulation by tumor cells.

18. The cell of claim 15 or 16, wherein the increase in cytokine release comprises an increase in granulocyte-macrophage colony-stimulating factor (GMCSF), soluble CD137 (sCD137), INF-g, Granzyme A, interleukin 13 (IL-13), Granzyme B, soluble FAS cell surface death receptor (sFas), interleukin 6 (IL-6), soluble FAS cell surface death receptor ligand (sFasL), macrophage inflammatory protein-1 alpha (MIP-1a), macrophage inflammatory protein-1 beta (MIP-1b), TNF-α, and/or Perforin, in response to stimulation by tumor cells.

19. The cell of any one of claims 1-18, wherein the mutation provides an enhanced activated and/or cytotoxic phenotype for the engineered cell relative to a control non-engineered cell.

20. The cell of any one of claims 1-19, wherein the mutation provides an enhanced activated and/or cytotoxic phenotype for the engineered cell relative to a control non-engineered cell, wherein the enhanced activated and/or cytotoxic phenotype is associated with one or more of GSEA identified pathways: G2M checkpoint, E2F targets, P53 pathway, Mitotic spindle, MYC, MTORC1, Androgen Response, Unfolded Protein Response, Spermatogenesis, Heme Metabolism, TNFalpha signaling, Protein Secretion, Apoptosis, Oxidative Phosphorylation, DNA Repair, UV Response, and/or Estrogen Response Early.

21. The cell of any one of claims 1-20, wherein the mutation leads to upregulation of G2M, E2F, MYC, MTORC1, oxidative phosphorylation, and/or TNFa signaling.

22. The cell of any one of claims 1-21, wherein the mutation provides an enhanced proliferative capacity and/or persistence phenotype for the engineered cell relative to a control non-engineered cell.

23. The cell of claim 22, wherein the enhanced proliferative capacity and/or persistence occurs in the absence of stimulation by exogenous interleukin 2 (IL-2).

24. The cell of claim 22 or 23, wherein the enhanced proliferative capacity and/or persistence does not result in autonomous growth.

25. The cell of any one of claims 1-24, wherein the mutation provides an enhanced metabolic fitness phenotype for the engineered cell relative to a control non-engineered cell.

26. The cell of claim 25, wherein the enhanced metabolic fitness is a higher glycolytic capacity and/or improved oxygen consumption rate (OCR).

27. The cell of any one of claims 1-26, wherein the mutation provides an increased chromatic accessibility across the genome when the cells are comprised in an acidic microenvironment.

28. The cell of any one of claims 1-27, wherein the endogenous gene is CREM.

29. The cell of claim 28, wherein the CREM mutation results in a decrease in expression of CREM RNA isoforms CREM-228 (ICER), CREM-207, CREM-230, CREM-211, CREM-213, CREM-239, CREM-201, CREM-232, CREM-217, and/or CREM-225.

30. The cell of claim 28 or 29, wherein the CREM mutation results in an increase in expression of CREM RNA isoform CREM-218.

31. The cell of any one of claims 28-30, wherein the CREM mutation is a result of exposure of the cell to a polynucleotide comprising the sequence of SEQ ID NO: 140 and/or SEQ ID NO: 142.

32. The cell of any one of claims 28-31, wherein the CREM mutation results in a decrease in one or more CREM protein isoforms by greater than 60%.

33. The cell of any one of claims 28-32, wherein the CREM mutation results in a decrease in one or more CREM protein isoforms by greater than 80%.

34. The cell of any one of claims 1-33, wherein the cell has further been conditioned to acidic microenvironments by contacting the cell ex vivo with acidic stimuli.

35. The cell of claim 34, wherein the acidic stimuli is provided at a concentration of greater than or equal to about 2 to 3 mM, optionally greater than or equal to about 2.5 mM.

36. The cell of any one of claims 34-35, wherein the conditioning to acidic microenvironments is by incremental and/or cumulative contacting with acidic stimuli.

37. The cell of any one of claims 34-36, wherein the cells are conditioned over a period of at least about 10 to 18 days, optionally at least about 14 days.

38. The cell of claim 36 or 37, wherein cell conditioning comprises addition of acidic stimuli about every 48-72 hours, optionally about every 48 hours.

39. The cell of any one of claims 34-37, wherein the acidic stimuli comprises or consists essentially of lactic acid.

40. The cell of any one of claims 34-39, wherein the cell is conditioned to acidic microenvironments of less than or equal to about pH 6.0.

41. The cell of any one of claims 1-40, wherein the cell is a T cell, natural killer (NK) cell, NK T cell, macrophage, B cell, invariant NKT cells, gamma delta T cells, MSCs, tumor-infiltrating lymphocyte, or dendritic cell.

42. The cell of any one of claims 1-41, wherein the cell is a NK cell derived from cord blood (CB), peripheral blood (PB), an NK cell line, bone marrow, a stem cell, or a mixture thereof.

43. The cell of claim 41 or 42, wherein the NK cell is derived from cord blood.

44. The cell of any one of claims 1-43, wherein the cell comprises one or more engineered receptors.

45. The cell of claim 44, wherein the one or more engineered receptors comprises an engineered antigen receptor that specifically targets an antigen.

46. The cell of claim 45, wherein the engineered antigen receptor is a chimeric antigen receptor (CAR) and/or a T cell receptor (TCR).

47. The cell of claim 46, wherein the engineered antigen receptor is a CAR.

48. The cell of any one of claims 45-47, wherein the antigen is a cancer antigen.

49. The cell of any one of claims 45-48, wherein the antigen is a solid tumor antigen.

50. The cell of any one of claims 45-49, wherein the antigen is selected from the group consisting of 5T4, 8H9, αvβ6 integrin, BCMA, B7-H3, B7-H6, CAIX, CA9, CD5, CD19, CD20, CD22, CD30, CD33, CD3δ, CD44, CD44v6, CD44v7/8, CD70, CD123, CD138, CD171, CEA, CSPG4, CS1, CLL1, CD99, DLL3, EGFR, EGFR family including ErbB2 (HER2), EGFRvIII, EGP2, EGP40, ERBB3, ERBB4, ErbB3/4, EPCAM, EphA2, EpCAM, FAP, FBP, fetal AchR, FRα, GD2, GD3, Glypican-3 (GPC3), HLA-A1+MAGE1, HLA-A1+NY-ESO-1, IL-11Rx, IL-13Rα2, Lambda, Lewis-Y, LICAM, Kappa, KDR, MCSP, Mesothelin, Muc1, Muc16, NCAM, NKG2D Ligands, NY-ESO-1, PRAME, PSC1, PSCA, PSMA, ROR1, SP17, Survivin, TAG72, TROP2, TEMs, HMW-MAA, VEGFR2, and a combination thereof.

51. The cell of any one of claims 45-50, wherein the antigen comprises TROP2 and/or CD70.

52. The cell of any one of claims 44-51, wherein the one or more engineered receptors comprises a cytokine receptor, chemokine receptor, homing receptor, or a combination thereof.

53. The cell of any one of claims 1-52, wherein the cell comprises expression of one or more exogenous chemokines and/or one or more cytokines.

54. The cell of claim 53, wherein the cytokine is IL-15, IL-12, IL-21, IL-2, IL-18, IL-7, or a combination thereof.

55. The cell of claim 54, wherein the cytokine is IL-15.

56. The cell of any one of claims 1-55, wherein the cell comprises a suicide gene.

57. The cell of any one of claims 1-56, wherein the endogenous gene is mutated as a result of homologous recombination or non-homologous recombination.

58. The cell of any one of claims 1-57, wherein the endogenous gene is mutated by an endonuclease.

59. The cell of claim 58, wherein the endonuclease is an RNA guided endonuclease.

60. The cell of claim 59, wherein the RNA guided endonuclease is CRISPR-Cas9.

61. The cell of any one of claims 1-60, wherein the cell comprises one or more additional mutations in one or more genes, wherein the gene is selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD3δ, CD40, IL21R, ICAM1, CD95, CD80, CD86, IL10R, CD5, GR, and CD7.

62. A population of cells according to any one of claims 1-61.

63. The population of cells of claim 62, wherein the population is comprised in a pharmaceutically acceptable excipient.

64. A method of treating cancer in an individual in need thereof, comprising the step of administering a therapeutically effective amount of the population of cells of claim 62 or 63 to the individual.

65. The method of claim 64, wherein the cells are autologous, allogeneic, or xenogeneic with respect to the individual.

66. The method of claim 64 or 65, wherein the cells are allogeneic with respect to the individual.

67. The method of any one of claims 64-66, wherein the cancer comprises a solid tumor.

68. The method of any one of claims 64-66, wherein the cancer does not comprise a solid tumor.

69. The method of any one of claims 64-68, wherein the cancer is of the lung, brain, breast, blood, skin, pancreas, liver, colon, head and neck, kidney, thyroid, stomach, spleen, gallbladder, bone, ovary, testes, endometrium, prostate, rectum, anus, and/or cervix.

70. The method of any one of claims 64-69, wherein the individual is a mammal.

71. The method of claim 70, wherein the individual is a human, dog, cat, horse, cow, sheep, pig, or rodent.

72. The method of claim 70 or 71, wherein the individual is a human.

73. The method of any one of claims 64-72, wherein the individual is administered an additional cancer therapy.

74. The method of claim 73, wherein the additional cancer therapy is surgery, radiation, chemotherapy, hormone therapy, immunotherapy, or a combination thereof.

75. The method of any one of claims 64-74, further comprising the step of diagnosing cancer in the individual.

76. A method of engineering an immune effector cell, comprising mutating an endogenous CAMP response element modulator (CREM), G-protein coupled receptor 4 (GPR4), G-protein coupled receptor 31 (GPR31), G-protein coupled receptor 68 (GPR68), G-protein coupled receptor 81 (GPR81), G-protein coupled receptor 132 (GPR132), G-protein coupled receptor 151 (GPR151), inducible cAMP early repressor (ICER), and/or cyclic AMP-responsive element-binding protein 1 (CREB1) gene of the cell.

77. The method of claim 76, wherein the mutating generates a partial or complete loss of function, and/or knock-out (KO) mutation.

78. The method of claim 76 or 77, wherein the mutating reduces or inhibits transcription or post-transcriptional processing of one or more mRNA isoforms encoded by the mutated endogenous gene relative to a non-mutated locus encoding the same endogenous gene.

79. The method of claim 76, wherein the mutating generates a neomorphic or gain of function mutation.

80. The method of claim 76 or 79, wherein the mutating increases transcription or post-transcriptional processing of one or more mRNA isoforms encoded by the mutated endogenous gene relative to a non-mutated locus encoding the same endogenous gene.

81. The method of any one of claims 76-80, wherein the mutating generates a modified mRNA isoform population encoded by the mutated endogenous gene relative to a representative mRNA population encoded by a non-mutated locus encoding the same endogenous gene.

82. The method of any one of claims 76-79, wherein the mutating generates a modified protein isoform population encoded by the mutated endogenous gene relative to a representative protein population encoded by a non-mutated locus encoding the same endogenous gene.

83. The method of any one of claims 76-82, wherein the mutating generates a knock-out (KO) mutation in the endogenous gene of the cell.

84. The method of any one of claims 76-82, wherein the mutating generates a homozygous mutation in the endogenous gene of the cell.

85. The method of any one of claims 76-82, wherein the mutating generates a heterozygous mutation in the endogenous gene of the cell.

86. The method of any one of claims 76-85, wherein the mutating generates an improved cytotoxicity of the engineered cell in an acidic microenvironment and/or a tumor microenvironment (TME) relative to a control non-engineered cell.

87. The method of any one of claims 76-86, wherein the mutating generates an improved cytotoxicity of the engineered cell in an acidic microenvironment with a pH less than or equal to about 7.0 relative to control non-engineered cell.

88. The method of any one of claims 76-87, wherein the mutating generates an improved cytotoxicity of the engineered cell in an acidic microenvironment with a pH less than or equal to about 5.9 relative to a control non-engineered cell.

89. The method of any one of claims 76-88, wherein the mutating results in improved cytotoxicity of the engineered cell in an acidic microenvironment characterized by increased levels of lactic acid relative to a non-acidic microenvironment.

90. The method of any one of claims 76-89, wherein the mutating generates an enhanced polyfunctionality of the engineered cell relative to a control non-engineered cell in response to stimulation by tumor cells.

91. The method of claim 90, wherein the enhanced polyfunctionality is evidenced by an increase in cytokine release in response to stimulation by tumor cells.

92. The method of claim 91, wherein the increase in cytokine release comprises an increase in interferon gamma (IFN-g), tumor necrosis factor alpha (TNF-a), and/or the degranulation marker CD107a, in response to stimulation by tumor cells.

93. The method of claim 90 or 91, wherein the increase in cytokine release comprises an increase in granulocyte-macrophage colony-stimulating factor (GMCSF), soluble CD137 (sCD137), INF-g, Granzyme A, interleukin 13 (IL-13), Granzyme B, soluble FAS cell surface death receptor (sFas), interleukin 6 (IL-6), soluble FAS cell surface death receptor ligand (sFasL), macrophage inflammatory protein-1 alpha (MIP-1a), macrophage inflammatory protein-1 beta (MIP-1b), TNF-α, and/or Perforin, in response to stimulation by tumor cells.

94. The method of any one of claims 76-93, wherein the mutating generates an enhanced activated and/or cytotoxic phenotype for the engineered cell relative to a control non-engineered cell.

95. The method of any one of claims 76-93, wherein the mutating leads to an enhanced activated and/or cytotoxic phenotype for the engineered cell relative to a control non-engineered cell, wherein the enhanced activated and/or cytotoxic phenotype is associated with one or more of GSEA identified pathways: G2M checkpoint, E2F targets, P53 pathway, Mitotic spindle, MYC, MTORC1, Androgen Response, Unfolded Protein Response, Spermatogenesis, Heme Metabolism, TNFalpha signaling, Protein Secretion, Apoptosis, Oxidative Phosphorylation, DNA Repair, UV Response, and/or Estrogen Response Early.

96. The method of any one of claims 76-95, wherein the mutating leads to upregulation of G2M, E2F, MYC, MTORC1, oxidative phosphorylation, and/or TNFa signaling.

97. The method of any one of claims 76-96, wherein the mutating generates an enhanced proliferative capacity and/or persistence phenotype for the engineered cell relative to a control non-engineered cell.

98. The method of claim 97, wherein the enhanced proliferative capacity and/or persistence occurs in the absence of stimulation by exogenous interleukin 2 (IL-2).

99. The method of claim 97 or 98, wherein the enhanced proliferative capacity and/or persistence does not result in autonomous growth.

100. The method of any one of claims 76-99, wherein the mutating generates an enhanced metabolic fitness phenotype for the engineered cell relative to a control non-engineered cell.

101. The method of claim 100, wherein the enhanced metabolic fitness is a higher glycolytic capacity and/or improved oxygen consumption rate (OCR).

102. The method of any one of claims 76-101, wherein the mutating provides an increased chromatic accessibility across the genome when the cells are comprised in an acidic microenvironment.

103. The method of any one of claims 76-102, wherein the endogenous gene is CREM.

104. The method of claim 103, wherein the mutating of CREM results in a decrease in expression of CREM RNA isoforms CREM-228 (ICER), CREM-207, CREM-230, CREM-211, CREM-213, CREM-239, CREM-201, CREM-232, CREM-217, and/or CREM-225.

105. The method of claim 103 or 104, wherein the mutating of CREM results in an increase in expression of CREM RNA isoform CREM-218.

106. The method of any one of claims 103-105, wherein the mutating of CREM comprises exposure of the cell to a polynucleotide comprising the sequence of SEQ ID NO: 140 and/or SEQ ID NO: 142.

107. The method of any one of claims 103-106, wherein the mutating of CREM generates a decrease in one or more CREM protein isoforms by greater than 60%.

108. The method of any one of claims 103-107, wherein the mutating of CREM generates a decrease in one or more CREM protein isoforms by greater than 80%.

109. The method of any one of claims 76-108, further comprising conditioning the cell to acidic microenvironments by contacting the cell ex vivo with acidic stimuli.

110. The method of claim 109, comprising providing the acidic stimuli at a concentration of greater than or equal to about 2 to 3 mM, optionally greater than or equal to about 2.5 mM.

111. The method of claim 109 or 110, wherein the conditioning to acidic microenvironments is by incremental and/or cumulative contacting with acidic stimuli.

112. The method of any one of claims 109-111, wherein the conditioning is over a period of at least about 10 to 18 days, optionally at least about 14 days.

113. The method of any one of claims 109-112, wherein the conditioning comprises addition of acidic stimuli about every 48-72 hours, optionally about every 48 hours.

114. The method of any one of claims 109-113, wherein the acidic stimuli comprises or consists essentially of lactic acid.

115. The method of any one of claims 109-114, wherein the conditioning is to acidic microenvironments of less than or equal to about pH 6.0.

116. The method of any one of claims 76-115, wherein the cell is a T cell, natural killer (NK) cell, NK T cell, macrophage, B cell, invariant NKT cells, gamma delta T cells, MSCs, tumor-infiltrating lymphocyte, or dendritic cell.

117. The method of any one of claims 76-116, wherein the cell is a NK cell derived from cord blood (CB), peripheral blood (PB), an NK cell line, bone marrow, a stem cell, or a mixture thereof.

118. The method of claim 116 or 117, wherein the NK cell is derived from cord blood.

119. The method of any one of claims 76-118, wherein the cell comprises one or more engineered receptors that specifically binds an antigen.

120. The method of claim 119, wherein the one or more engineered receptors comprises an engineered antigen receptor.

121. The method of claim 120, wherein the engineered antigen receptor is a chimeric antigen receptor (CAR) and/or a T cell receptor (TCR).

122. The method of claim 121, wherein the engineered antigen receptor is a CAR.

123. The method of any one of claims 120-122, wherein the antigen is a cancer antigen.

124. The method of any one of claims 120-123, wherein the antigen is a solid tumor antigen.

125. The method of any one of claims 120-124, wherein the antigen is selected from the group consisting of 5T4, 8H9, αvβ6 integrin, BCMA, B7-H3, B7-H6, CAIX, CA9, CD5, CD19, CD20, CD22, CD30, CD33, CD3δ, CD44, CD44v6, CD44v7/8, CD70, CD123, CD138, CD171, CEA, CSPG4, CS1, CLL1, CD99, DLL3, EGFR, EGFR family including ErbB2 (HER2), EGFRVIII, EGP2, EGP40, ERBB3, ERBB4, ErbB3/4, EPCAM, EphA2, EpCAM, FAP, FBP, fetal AchR, FRα, GD2, GD3, Glypican-3 (GPC3), HLA-A1+MAGE1, HLA-A1+NY-ESO-1, IL-11Rα, IL-13Rα2, Lambda, Lewis-Y, LICAM, Kappa, KDR, MCSP, Mesothelin, Muc1, Muc16, NCAM, NKG2D Ligands, NY-ESO-1, PRAME, PSC1, PSCA, PSMA, ROR1, SP17, Survivin, TAG72, TROP2, TEMs, HMW-MAA, VEGFR2, and a combination thereof.

126. The method of any one of claims 120-125, wherein the antigen comprises TROP2 and/or CD70.

127. The method of any one of claims 119-126, wherein the one or more engineered receptors comprises a cytokine receptor, chemokine receptor, homing receptor, or a combination thereof.

128. The method of any one of claims 76-127, wherein the cell comprises expression of one or more exogenous chemokines and/or one or more cytokines.

129. The method of claim 128, wherein the cytokine is IL-15, IL-12, IL-21, IL-2, IL-18, IL-7, or a combination thereof.

130. The method of claim 129, wherein the cytokine is IL-15.

131. The method of any one of claims 76-130, wherein the cell comprises a suicide gene.

132. The method of any one of claims 76-131, wherein the mutating of the endogenous gene is comprises homologous recombination or non-homologous recombination.

133. The method of any one of claims 76-132, wherein the mutating of the endogenous gene is mediated by an endonuclease.

134. The method of claim 133, wherein the endonuclease is an RNA guided endonuclease.

135. The method of claim 134, wherein the RNA guided endonuclease is CRISPR-Cas9.

136. The method of any one of claims 76-135, wherein the cell comprises one or more additional mutations in one or more genes, wherein the gene is selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD3δ, CD40, IL21R, ICAM1, CD95, CD80, CD86, IL10R, CD5, GR, and CD7.

137. An engineered natural killer (NK) cell, wherein the cell comprises an engineered partial or complete loss of function, and/or knock-out (KO) mutation in an endogenous cAMP response element modulator (CREM) gene in the cell.

138. The cell of claim 137, wherein the cell comprises a knock-out (KO) mutation in the endogenous CREM gene in the cell.

139. The cell of claim 138, wherein the cell comprises a homozygous knock-out (KO) mutation in the endogenous CREM gene.

140. The cell of claim 138, wherein the cell comprises heterozygous KO mutation in the endogenous CREM gene in the cell.

141. The cell of claim 137, wherein the mutation results in improved cytotoxicity of the engineered cell relative to a reference cell lacking the mutation.

142. The cell of claim 137, wherein the mutation results in improved cytotoxicity of the engineered cell in an acidic microenvironment and/or a tumor microenvironment (TME) relative to a control non-engineered cell.

143. The cell of claim 137, wherein the mutation results in improved cytotoxicity of the engineered cell in an acidic microenvironment with a pH less than or equal to about 7.0 relative to control non-engineered cell.

144. The cell of claim 137, wherein the mutation results in improved cytotoxicity of the engineered cell in an acidic microenvironment with a pH less than or equal to about 5.9 relative to a control non-engineered cell.

145. The cell of claim 137, wherein the mutation results in improved cytotoxicity of the engineered cell in an acidic environment characterized by increased levels of lactic acid relative to a non-acidic microenvironment.

146. The cell of claim 137, wherein the mutation results in enhanced polyfunctionality of the engineered cell relative to a control non-engineered cell in response to stimulation by tumor cells.

147. The cell of claim 146, wherein the enhanced polyfunctionality is evidenced by an increase in cytokine release in response to stimulation by tumor cells.

148. The cell of claim 137, wherein the increase in cytokine release comprises an increase in interferon gamma (IFN-g), tumor necrosis factor alpha (TNF-a), and/or the degranulation marker CD107a, in response to stimulation by tumor cells.

149. The cell of claim 148, wherein the increase in cytokine release comprises an increase in granulocyte-macrophage colony-stimulating factor (GMCSF), soluble CD137 (sCD137), INF-g, Granzyme A, interleukin 13 (IL-13), Granzyme B, soluble FAS cell surface death receptor (sFas), interleukin 6 (IL-6), soluble FAS cell surface death receptor ligand (sFasL), macrophage inflammatory protein-1 alpha (MIP-1a), macrophage inflammatory protein-1 beta (MIP-1b), TNF-α, and/or Perforin, in response to stimulation by tumor cells.

150. The cell of claim 137, wherein the mutation provides an enhanced activated and/or cytotoxic phenotype for the engineered cell relative to a control non-engineered cell.

151. The cell of claim 150, wherein the mutation provides an enhanced activated and/or cytotoxic phenotype for the engineered cell relative to a control non-engineered cell, wherein the enhanced activated and/or cytotoxic phenotype is associated with one or more of GSEA identified pathways: G2M checkpoint, E2F targets, P53 pathway, Mitotic spindle, MYC, MTORC1, Androgen Response, Unfolded Protein Response, Spermatogenesis, Heme Metabolism, TNFalpha signaling, Protein Secretion, Apoptosis, Oxidative Phosphorylation, DNA Repair, UV Response, and/or Estrogen Response Early.

152. The cell of claim 137, wherein the mutation provides for upregulation of G2M, E2F, MYC, MTORC1, oxidative phosphorylation, and/or TNFa signaling.

153. The cell of claim 137, wherein the mutation provides an enhanced proliferative capacity and/or persistence phenotype for the engineered cell relative to a control non-engineered cell.

154. The cell of claim 153, wherein the enhanced proliferative capacity and/or persistence occurs in the absence of stimulation by exogenous interleukin 2 (IL-2).

155. The cell of claim 153 or 154, wherein the enhanced proliferative capacity and/or persistence does not result in autonomous growth.

156. The cell of claim 137, wherein the mutation provides an enhanced metabolic fitness phenotype for the engineered cell relative to a control non-engineered cell.

157. The cell of claim 137, wherein the enhanced metabolic fitness is a higher glycolytic capacity and/or improved oxygen consumption rate (OCR).

158. The cell of claim 137, wherein the mutation provides an increased chromatic accessibility across the genome when the cells are comprised in an acidic environment.

159. The cell of claim 137, wherein the CREM mutation results in a decrease in expression of CREM RNA isoforms CREM-228 (ICER), CREM-207, CREM-230, CREM-211, CREM-213, CREM-239, CREM-201, CREM-232, CREM-217, and/or CREM-225

160. The cell of claim 137, wherein the CREM mutation is a result of exposure of the cell to a polynucleotide comprising the sequence of SEQ ID NO: 140 and/or SEQ ID NO: 142.

161. The cell of claim 137, wherein the CREM mutation results in a decrease in CREM protein isoforms by greater than 60%; or wherein the CREM mutation results in a decrease in CREM protein isoforms by greater than 80%.

162. The cell of claim 137, wherein the cell is derived from cord blood (CB).

163. The cell of claim 137, wherein the cell is derived from peripheral blood (PB).

164. The cell of claim 137, wherein the cell is derived from a stem cell.

165. The cell of claim 137, wherein the cells comprises an engineered receptor.

166. The cell of claim 137, wherein the cell comprises a T cell receptor (TCR).

167. The cell of claim 137, wherein the cell comprises a chimeric antigen receptor (CAR).

168. The cell of claim 165, wherein the engineered receptor specifically binds an antigen selected from the group consisting of 5T4, 8H9, αvβ6 integrin, BCMA, B7-H3, B7-H6, CAIX, CA9, CD5, CD19, CD20, CD22, CD30, CD33, CD3δ, CD44, CD44v6, CD44v7/8, CD70, CD123, CD138, CD171, CEA, CSPG4, CS1, CLL1, CD99, DLL3, EGFR, EGFR family including ErbB2 (HER2), EGFRVIII, EGP2, EGP40, ERBB3, ERBB4, ErbB3/4, EPCAM, EphA2, EpCAM, FAP, FBP, fetal AchR, FRα, GD2, GD3, Glypican-3 (GPC3), HLA-A1+MAGE1, HLA-A1+NY-ESO-1, IL-11Rα, IL-13Rα2, Lambda, Lewis-Y, LICAM, Kappa, KDR, MCSP, Mesothelin, Muc1, Muc16, NCAM, NKG2D Ligands, NY-ESO-1, PRAME, PSC1, PSCA, PSMA, ROR1, SP17, Survivin, TAG72, TROP2, TEMs, HMW-MAA, VEGFR2, and a combination thereof.

169. The cell of claim 168, wherein the engineered receptor specifically binds TROP2.

170. The cell of claim 168, wherein the engineered receptor specifically binds CD70.

171. A population of cells according to any one of claims 137-170

172. A pharmaceutical composition comprising the cell of any one of claim 137-170, and optionally a pharmaceutically acceptable excipient.

173. A method of treating cancer in an individual in need thereof, comprising the step of administering a therapeutically effective amount of the cell of any one of claims 137-170, the population of claim 171, or the pharmaceutical composition of claim 172 to the individual.

174. The method of claim 173, wherein the cancer comprises a solid tumor.

175. The method of claim 173, wherein the cancer is of the lung, brain, breast, blood, skin, pancreas, liver, colon, head and neck, kidney, thyroid, stomach, spleen, gallbladder, bone, ovary, testes, endometrium, prostate, rectum, anus, and/or cervix.

176. The method of claim 173, wherein the individual is a mammal.

177. The method of claim 173, wherein the individual is a human.

178. A method of engineering a cell according to any one of claims 137-170.

179. A method of killing a cancer cell, comprising contacting the cancer cell with an engineered NK cell according to any one of claims 137-170.


SEQUENCE LISTING (193 sequences)

SEQ ID NO: 1 - DNA, 23 bp, synthetic construct
  misc_feature 1..23: note = CDS of CREM forward primer
  source 1..23: mol_type = other DNA
tgaatgaactgtcctctgatgtg

SEQ ID NO: 2 - DNA, 27 bp, synthetic construct
  misc_feature 1..27: note = CDS of CREM reverse primer
  source 1..27: mol_type = other DNA
cctgagttgcttcaatataactagaga

SEQ ID NO: 3 - DNA, 20 bp, synthetic construct
  misc_feature 1..20: note = GPR4 crRNA
  source 1..20: mol_type = other DNA
agcccacgaacacccgatag

SEQ ID NO: 4 - DNA, 20 bp, synthetic construct
  misc_feature 1..20: note = GPR4 crRNA
  source 1..20: mol_type = other DNA
catctcggtggaccgctacc

SEQ ID NO: 5 - DNA, 20 bp, synthetic construct
  misc_feature 1..20: note = GPR4 crRNA
  source 1..20: mol_type = other DNA
tacatctttgtcatcggcgt

SEQ ID NO: 6 - DNA, 20 bp, synthetic construct
  misc_feature 1..20: note = GPR4 crRNA
  source 1..20: mol_type = other DNA
ggtctcggaagagctcgtca

SEQ ID NO: 7 - DNA, 20 bp, synthetic construct
  misc_feature 1..20: note = GPR4 crRNA
  source 1..20: mol_type = other DNA
acttcctgcaccacgacaac

SEQ ID NO: 8 - DNA, 20 bp, synthetic construct
  misc_feature 1..20: note = GPR4 crRNA
  source 1..20: mol_type = other DNA
gaccacggagctcacggcca

SEQ ID NO: 9 - DNA, 20 bp, synthetic construct
  misc_feature 1..20: note = GPR4 crRNA
  source 1..20: mol_type = other DNA
atgggcaaccacacgtggga

SEQ ID NO: 10 - DNA, 20 bp, synthetic construct
  misc_feature 1..20: note = GPR4 crRNA
  source 1..20: mol_type = other DNA
aggaagtagtccacccacag

SEQ ID NO: 11 - DNA, 20 bp, synthetic construct
  misc_feature 1..20: note = GPR4 crRNA
  source 1..20: mol_type = other DNA
aacctctatcgggtgttcgt

SEQ ID NO: 12 - DNA, 20 bp, synthetic construct
  misc_feature 1..20: note = GPR4 crRNA
  source 1..20: mol_type = other DNA
cgccgatgacaaagatgtag

SEQ ID NO: 13 - DNA, 20 bp, synthetic construct
  misc_feature 1..20: note = GPR4 crRNA
  source 1..20: mol_type = other DNA
cacggagctgggcgccaact

SEQ ID NO: 14 - DNA, 20 bp, synthetic construct
  misc_feature 1..20: note = GPR4 crRNA
  source 1..20: mol_type = other DNA
caccacgacaactggatcca

SEQ ID NO: 15 - DNA, 20 bp, synthetic construct
  misc_feature 1..20: note = GPR4 crRNA
  source 1..20: mol_type = other DNA
tggcctggatgaacctctat

SEQ ID NO: 16 - DNA, 20 bp, synthetic construct
  misc_feature 1..20: note = GPR4 crRNA
  source 1..20: mol_type = other DNA
gacaactggatccacggccc

SEQ ID NO: 17 - DNA, 20 bp, synthetic construct
  misc_feature 1..20: note = GPR4 crRNA
  source 1..20: mol_type = other DNA
acaactggatccacggcccc

SEQ ID NO: 18 - DNA, 20 bp, synthetic construct
  misc_feature 1..20: note = GPR4 crRNA
  source 1..20: mol_type = other DNA
ggaagagctcgtcatggaac

SEQ ID NO: 19 - DNA, 20 bp, synthetic construct
  misc_feature 1..20: note = GPR4 crRNA
  source 1..20: mol_type = other DNA
ggtccaccgagatgcagcac

SEQ ID NO: 20 - DNA, 20 bp, synthetic construct
  misc_feature 1..20: note = GPR4 crRNA
  source 1..20: mol_type = other DNA
gcatgagcgcccacgggaag

SEQ ID NO: 21 - DNA, 20 bp, synthetic construct
  misc_feature 1..20: note = GPR4 crRNA
  source 1..20: mol_type = other DNA
gaagagctcgtcatggaaca

SEQ ID NO: 22 - DNA, 20 bp, synthetic construct
  misc_feature 1..20: note = GPR4 crRNA
  source 1..20: mol_type = other DNA
gatgaacccaaagagcttgc

SEQ ID NO: 23 - DNA, 20 bp, synthetic construct
  misc_feature 1..20: note = GPR4 crRNA
  source 1..20: mol_type = other DNA
ggacagcaagagcacgtgat

SEQ ID NO: 24 - DNA, 20 bp, synthetic construct
  misc_feature 1..20: note = GPR4 crRNA
  source 1..20: mol_type = other DNA
gacagcaagagcacgtgata

SEQ ID NO: 25 - DNA, 20 bp, synthetic construct
  misc_feature 1..20: note = GPR4 crRNA
  source 1..20: mol_type = other DNA
tttcaccagcctcaactgtg

SEQ ID NO: 26 - DNA, 20 bp, synthetic construct
  misc_feature 1..20: note = GPR31 crRNA
  source 1..20: mol_type = other DNA
gcaggttgaccttaagccga

SEQ ID NO: 27 - DNA, 20 bp, synthetic construct
  misc_feature 1..20: note = GPR31 crRNA
  source 1..20: mol_type = other DNA
gtaccggtccaaagccacgg

SEQ ID NO: 28 - DNA, 20 bp, synthetic construct
  misc_feature 1..20: note = GPR31 crRNA
  source 1..20: mol_type = other DNA
tccacaccctgacccggaac

SEQ ID NO: 29 - DNA, 20 bp, synthetic construct
  misc_feature 1..20: note = GPR31 crRNA
  source 1..20: mol_type = other DNA
ggtgccacagtttctactcc

SEQ ID NO: 30 - DNA, 20 bp, synthetic construct
  misc_feature 1..20: note = GPR31 crRNA
  source 1..20: mol_type = other DNA
ctgcattgcagaacacgatg

SEQ ID NO: 31 - DNA, 20 bp, synthetic construct
  misc_feature 1..20: note = GPR31 crRNA
  source 1..20: mol_type = other DNA
tgtggcacctggtggagttc

SEQ ID NO: 32 - DNA, 20 bp, synthetic construct
  misc_feature 1..20: note = GPR31 crRNA
  source 1..20: mol_type = other DNA
agagatgagcaagcccgggc

SEQ ID NO: 33 - DNA, 20 bp, synthetic construct
  misc_feature 1..20: note = GPR31 crRNA
  source 1..20: mol_type = other DNA
agcaggttgaccttaagccg

SEQ ID NO: 34 - DNA, 20 bp, synthetic construct
  misc_feature 1..20: note = GPR31 crRNA
  source 1..20: mol_type = other DNA
gccgagggtggaccacacgg

SEQ ID NO: 35 - DNA, 20 bp, synthetic construct
  misc_feature 1..20: note = GPR31 crRNA
  source 1..20: mol_type = other DNA
ggtggaccacacggaggtac

SEQ ID NO: 36 - DNA, 20 bp, synthetic construct
  misc_feature 1..20: note = GPR31 crRNA
  source 1..20: mol_type = other DNA
acctccgtgtggtccaccct

SEQ ID NO: 37 - DNA, 20 bp, synthetic construct
  misc_feature 1..20: note = GPR31 crRNA
  source 1..20: mol_type = other DNA
taagccgagggtggaccaca

SEQ ID NO: 38 - DNA, 20 bp, synthetic construct
  misc_feature 1..20: note = GPR31 crRNA
  source 1..20: mol_type = other DNA
acggcttccacaccctgacc

SEQ ID NO: 39 - DNA, 20 bp, synthetic construct
  misc_feature 1..20: note = GPR31 crRNA
  source 1..20: mol_type = other DNA
gaggtaccggtccaaagcca

SEQ ID NO: 40 - DNA, 20 bp, synthetic construct
  misc_feature 1..20: note = GPR31 crRNA
  source 1..20: mol_type = other DNA
ggtcaacctgctgtctcctc

SEQ ID NO: 41 - DNA, 20 bp, synthetic construct
  misc_feature 1..20: note = GPR31 crRNA
  source 1..20: mol_type = other DNA
cagaacacgatgaggccaaa

SEQ ID NO: 42 - DNA, 20 bp, synthetic construct
  misc_feature 1..20: note = GPR31 crRNA
  source 1..20: mol_type = other DNA
ggaaggccatccccacgctg

SEQ ID NO: 43 - DNA, 20 bp, synthetic construct
  misc_feature 1..20: note = GPR68 crRNA
  source 1..20: mol_type = other DNA
gatgaccacgctgacgccga

SEQ ID NO: 44 - DNA, 20 bp, synthetic construct
  misc_feature 1..20: note = GPR68 crRNA
  source 1..20: mol_type = other DNA
cacggtaacatagaccaccg

SEQ ID NO: 45 - DNA, 20 bp, synthetic construct
  misc_feature 1..20: note = GPR68 crRNA
  source 1..20: mol_type = other DNA
acgtgctgcagcacgacaac

SEQ ID NO: 46 - DNA, 20 bp, synthetic construct
  misc_feature 1..20: note = GPR68 crRNA
  source 1..20: mol_type = other DNA
gatcaaggcccggaacgagc

SEQ ID NO: 47 - DNA, 20 bp, synthetic construct
  misc_feature 1..20: note = GPR68 crRNA
  source 1..20: mol_type = other DNA
gagaacatctacatcagcgt

SEQ ID NO: 48 - DNA, 20 bp, synthetic construct
  misc_feature 1..20: note = GPR68 crRNA
  source 1..20: mol_type = other DNA
ggtagtgctcaaagcacacg

SEQ ID NO: 49 - DNA, 20 bp, synthetic construct
  misc_feature 1..20: note = GPR68 crRNA
  source 1..20: mol_type = other DNA
catctacttcctgatgcacg

SEQ ID NO: 50 - DNA, 20 bp, synthetic construct
  misc_feature 1..20: note = GPR68 crRNA
  source 1..20: mol_type = other DNA
gccgcaaggaccagatccag

SEQ ID NO: 51 - DNA, 20 bp, synthetic construct
  misc_feature 1..20: note = GPR68 crRNA
  source 1..20: mol_type = other DNA
caactactaccgcttcctgg

SEQ ID NO: 52 - DNA, 20 bp, synthetic construct
  misc_feature 1..20: note = GPR68 crRNA
  source 1..20: mol_type = other DNA
aactgcctgtccctctactt

SEQ ID NO: 53 - DNA, 20 bp, synthetic construct
  misc_feature 1..20: note = GPR68 crRNA
  source 1..20: mol_type = other DNA
gcacggtaacatagaccacc

SEQ ID NO: 54 - DNA, 20 bp, synthetic construct
  misc_feature 1..20: note = GPR68 crRNA
  source 1..20: mol_type = other DNA
ccataccatccaccagacgc

SEQ ID NO: 55 - DNA, 20 bp, synthetic construct
  misc_feature 1..20: note = GPR68 crRNA
  source 1..20: mol_type = other DNA
agaagaggtcggccaccgtc

SEQ ID NO: 56 - DNA, 20 bp, synthetic construct
  misc_feature 1..20: note = GPR68 crRNA
  source 1..20: mol_type = other DNA
cagcacgacaactggtctca

SEQ ID NO: 57 - DNA, 20 bp, synthetic construct
  misc_feature 1..20: note = GPR68 crRNA
  source 1..20: mol_type = other DNA
gtacctgtgcaacctgacgg

SEQ ID NO: 58 - DNA, 20 bp, synthetic construct
  misc_feature 1..20: note = GPR68 crRNA
  source 1..20: mol_type = other DNA
agcacggtaacatagaccac

SEQ ID NO: 59 - DNA, 20 bp, synthetic construct
  misc_feature 1..20: note = GPR68 crRNA
  source 1..20: mol_type = other DNA
ctacttcctgatgcacgagg

SEQ ID NO: 60 - DNA, 20 bp, synthetic construct
  misc_feature 1..20: note = GPR68 crRNA
  source 1..20: mol_type = other DNA
ctttgagcactaccccatcc

SEQ ID NO: 61 - DNA, 20 bp, synthetic construct
  misc_feature 1..20: note = GPR68 crRNA
  source 1..20: mol_type = other DNA
cgtgtacctgtgcaacctga

SEQ ID NO: 62 - DNA, 20 bp, synthetic construct
  misc_feature 1..20: note = GPR68 crRNA
  source 1..20: mol_type = other DNA
ccaccagttccggaccctga

SEQ ID NO: 63 - DNA, 20 bp, synthetic construct
  misc_feature 1..20: note = GPR68 crRNA
  source 1..20: mol_type = other DNA
cgggaagcccaccaccagca

SEQ ID NO: 64 - DNA, 20 bp, synthetic construct
  misc_feature 1..20: note = GPR68 crRNA
  source 1..20: mol_type = other DNA
tgatgtagatgttctcgtac

SEQ ID NO: 65 - DNA, 20 bp, synthetic construct
  misc_feature 1..20: note = GPR68 crRNA
  source 1..20: mol_type = other DNA
ccagttccggaccctgaagg

SEQ ID NO: 66 - DNA, 20 bp, synthetic construct
  misc_feature 1..20: note = GPR68 crRNA
  source 1..20: mol_type = other DNA
gaaggggccacactcctcat

SEQ ID NO: 67 - DNA, 20 bp, synthetic construct
  misc_feature 1..20: note = GPR68 crRNA
  source 1..20: mol_type = other DNA
caaggaccagatccagcggc

SEQ ID NO: 68 - DNA, 20 bp, synthetic construct
  misc_feature 1..20: note = GPR68 crRNA
  source 1..20: mol_type = other DNA
gccctaccacgtgttgctgc

SEQ ID NO: 69 - DNA, 20 bp, synthetic construct
  misc_feature 1..20: note = GPR68 crRNA
  source 1..20: mol_type = other DNA
cgcagttgaagctggtgagc

SEQ ID NO: 70 - DNA, 20 bp, synthetic construct
  misc_feature 1..20: note = GPR68 crRNA
  source 1..20: mol_type = other DNA
tcgtcagcgagaccacccac

SEQ ID NO: 71 - DNA, 20 bp, synthetic construct
  misc_feature 1..20: note = GPR68 crRNA
  source 1..20: mol_type = other DNA
cgtcagcgagaccacccacc

SEQ ID NO: 72 - DNA, 20 bp, synthetic construct
  misc_feature 1..20: note = GPR68 crRNA
  source 1..20: mol_type = other DNA
cgagaccacccaccgggacc

SEQ ID NO: 73 - DNA, 20 bp, synthetic construct
  misc_feature 1..20: note = GPR68 crRNA
  source 1..20: mol_type = other DNA
gctgttgaccaagctccacc

SEQ ID NO: 74 - DNA, 20 bp, synthetic construct
  misc_feature 1..20: note = GPR68 crRNA
  source 1..20: mol_type = other DNA
gtggagcttggtcaacagct

SEQ ID NO: 75 - DNA, 20 bp, synthetic construct
  misc_feature 1..20: note = GPR81 crRNA
  source 1..20: mol_type = other DNA
gagcatcgtgttccttacgg

SEQ ID NO: 76 - DNA, 20 bp, synthetic construct
  misc_feature 1..20: note = GPR81 crRNA
  source 1..20: mol_type = other DNA
cgtgaagagccccactcggc

SEQ ID NO: 77 - DNA, 20 bp, synthetic construct
  misc_feature 1..20: note = GPR81 crRNA
  source 1..20: mol_type = other DNA
cggtgaacactatctccacc

SEQ ID NO: 78 - DNA, 20 bp, synthetic construct
  misc_feature 1..20: note = GPR81 crRNA
  source 1..20: mol_type = other DNA
ttcatcatggagtcggccaa

SEQ ID NO: 79 - DNA, 20 bp, synthetic construct
  misc_feature 1..20: note = GPR81 crRNA
  source 1..20: mol_type = other DNA
aggtaatagtctgtccgaaa

SEQ ID NO: 80 - DNA, 20 bp, synthetic construct
  misc_feature 1..20: note = GPR81 crRNA
  source 1..20: mol_type = other DNA
tgccaccaccatgatgaacc

SEQ ID NO: 81 - DNA, 20 bp, synthetic construct
  misc_feature 1..20: note = GPR81 crRNA
  source 1..20: mol_type = other DNA
cagccacggccaaattgaaa

SEQ ID NO: 82 - DNA, 20 bp, synthetic construct
  misc_feature 1..20: note = GPR81 crRNA
  source 1..20: mol_type = other DNA
ctggagttctttatgcccct

SEQ ID NO: 83 - DNA, 20 bp, synthetic construct
  misc_feature 1..20: note = GPR81 crRNA
  source 1..20: mol_type = other DNA
gcaaaagatacactgttccc

SEQ ID NO: 84 - DNA, 20 bp, synthetic construct
  misc_feature 1..20: note = GPR81 crRNA
  source 1..20: mol_type = other DNA
aaggccacaatgagcagcgg

SEQ ID NO: 85 - DNA, 20 bp, synthetic construct
  misc_feature 1..20: note = GPR81 crRNA
  source 1..20: mol_type = other DNA
cgtaaggaacacgatgctcc

SEQ ID NO: 86 - DNA, 20 bp, synthetic construct
  misc_feature 1..20: note = GPR81 crRNA
  source 1..20: mol_type = other DNA
ccgagtggggctcttcacgt

SEQ ID NO: 87 - DNA, 20 bp, synthetic construct
  misc_feature 1..20: note = GPR81 crRNA
  source 1..20: mol_type = other DNA
gaacactatctccacccggg

SEQ ID NO: 88 - DNA, 20 bp, synthetic construct
  misc_feature 1..20: note = GPR81 crRNA
  source 1..20: mol_type = other DNA
attacctcagacgtagacac

SEQ ID NO: 89 - DNA, 20 bp, synthetic construct
  misc_feature 1..20: note = Synthetic polynucleotide
  source 1..20: mol_type = other DNA
ggtgaacactatctccaccc

SEQ ID NO: 90 - DNA, 20 bp, synthetic construct
  misc_feature 1..20: note = Synthetic polynucleotide
  source 1..20: mol_type = other DNA
cactatctccacccgggtgg

SEQ ID NO: 91 - DNA, 20 bp, synthetic construct
  misc_feature 1..20: note = Synthetic polynucleotide
  source 1..20: mol_type = other DNA
ccaacgtgaagagccccact

SEQ ID NO: 92 - DNA, 20 bp, synthetic construct
  misc_feature 1..20: note = Synthetic polynucleotide
  source 1..20: mol_type = other DNA
ttacctcagacgtagacact

SEQ ID NO: 93 - DNA, 20 bp, synthetic construct
  misc_feature 1..20: note = Synthetic polynucleotide
  source 1..20: mol_type = other DNA
gtgaagagccccactcggca

SEQ ID NO: 94 - DNA, 20 bp, synthetic construct
  misc_feature 1..20: note = Synthetic polynucleotide
  source 1..20: mol_type = other DNA
agtggtccacccccaccacg

SEQ ID NO: 95 - DNA, 20 bp, synthetic construct
  misc_feature 1..20: note = Synthetic polynucleotide
  source 1..20: mol_type = other DNA
tactacgccaggttcaccgt

SEQ ID NO: 96 - DNA, 20 bp, synthetic construct
  misc_feature 1..20: note = Synthetic polynucleotide
  source 1..20: mol_type = other DNA
ccgcgctgtacaccacgacc

SEQ ID NO: 97 - DNA, 20 bp, synthetic construct
  misc_feature 1..20: note = Synthetic polynucleotide
  source 1..20: mol_type = other DNA
cgtacaccacggccacgaag

SEQ ID NO: 98 - DNA, 20 bp, synthetic construct
  misc_feature 1..20: note = Synthetic polynucleotide
  source 1..20: mol_type = other DNA
ggtgaagcactcggccatcg

SEQ ID NO: 99 - DNA, 20 bp, synthetic construct
  misc_feature 1..20: note = Synthetic polynucleotide
  source 1..20: mol_type = other DNA
ggtagtgaacgatcccgacg

SEQ ID NO: 100 - DNA, 20 bp, synthetic construct
  misc_feature 1..20: note = Synthetic polynucleotide
  source 1..20: mol_type = other DNA
ctctgcgagctgctgtacac

SEQ ID NO: 101 - DNA, 20 bp, synthetic construct
  misc_feature 1..20: note = Synthetic polynucleotide
  source 1..20: mol_type = other DNA
agtgaccaccactgccccgt

SEQ ID NO: 102 - DNA, 20 bp, synthetic construct
  misc_feature 1..20: note = Synthetic polynucleotide
  source 1..20: mol_type = other DNA
ggtactgcagggcaacgtgc

SEQ ID NO: 103 - DNA, 20 bp, synthetic construct
  misc_feature 1..20: note = Synthetic polynucleotide
  source 1..20: mol_type = other DNA
tcaccaaccaccggattttc

SEQ ID NO: 104 - DNA, 20 bp, synthetic construct
  misc_feature 1..20: note = Synthetic polynucleotide
  source 1..20: mol_type = other DNA
gatgtaggcggtcaccttgc

SEQ ID NO: 105 - DNA, 20 bp, synthetic construct
  misc_feature 1..20: note = Synthetic polynucleotide
  source 1..20: mol_type = other DNA
ttcgaaggacacgttgttgc

SEQ ID NO: 106 - DNA, 20 bp, synthetic construct
  misc_feature 1..20: note = Synthetic polynucleotide
  source 1..20: mol_type = other DNA
gaagatgacaaccaccgcga

SEQ ID NO: 107 - DNA, 20 bp, synthetic construct
  misc_feature 1..20: note = Synthetic polynucleotide
  source 1..20: mol_type = other DNA
acgtgtccttcgaagagagc

SEQ ID NO: 108 - DNA, 20 bp, synthetic construct
  misc_feature 1..20: note = Synthetic polynucleotide
  source 1..20: mol_type = other DNA
gactatcctgctctcttcga

SEQ ID NO: 109 - DNA, 20 bp, synthetic construct
  misc_feature 1..20: note = Synthetic polynucleotide
  source 1..20: mol_type = other DNA
tcactacccggtgttccaga

SEQ ID NO: 110 - DNA, 20 bp, synthetic construct
  misc_feature 1..20: note = Synthetic polynucleotide
  source 1..20: mol_type = other DNA
ggacacgttgttgcaggtct

SEQ ID NO: 111 - DNA, 20 bp, synthetic construct
  misc_feature 1..20: note = Synthetic polynucleotide
  source 1..20: mol_type = other DNA
gatggccgagtgcttcacct

SEQ ID NO: 112 - DNA, 20 bp, synthetic construct
  misc_feature 1..20: note = Synthetic polynucleotide
  source 1..20: mol_type = other DNA
tcatcgccttcaccaaccac

SEQ ID NO: 113 - DNA, 20 bp, synthetic construct
  misc_feature 1..20: note = Synthetic polynucleotide
  source 1..20: mol_type = other DNA
tgtcttccgtctggaacacc

SEQ ID NO: 114 - DNA, 20 bp, synthetic construct
  misc_feature 1..20: note = Synthetic polynucleotide
  source 1..20: mol_type = other DNA
ttgtcttccgtctggaacac

SEQ ID NO: 115 - DNA, 20 bp, synthetic construct
  misc_feature 1..20: note = Synthetic polynucleotide
  source 1..20: mol_type = other DNA
cctgtccacggtgaacggcg

SEQ ID NO: 116 - DNA, 20 bp, synthetic construct
  misc_feature 1..20: note = Synthetic polynucleotide
  source 1..20: mol_type = other DNA
gaagtgtccagaatccataa

SEQ ID NO: 117 - DNA, 20 bp, synthetic construct
  misc_feature 1..20: note = Synthetic polynucleotide
  source 1..20: mol_type = other DNA
tgtccagaatccataagggg

SEQ ID NO: 118 - DNA, 20 bp, synthetic construct
  misc_feature 1..20: note = Synthetic polynucleotide
  source 1..20: mol_type = other DNA
ccatgaagacagacgtcacc

SEQ ID NO: 119 - DNA, 20 bp, synthetic construct
  misc_feature 1..20: note = Synthetic polynucleotide
  source 1..20: mol_type = other DNA
ggtgaccctggtgggtgcac

SEQ ID NO: 120 - DNA, 20 bp, synthetic construct
  misc_feature 1..20: note = Synthetic polynucleotide
  source 1..20: mol_type = other DNA
tgctaaagaaccattccggc

SEQ ID NO: 121 - DNA, 20 bp, synthetic construct
  misc_feature 1..20: note = Synthetic polynucleotide
  source 1..20: mol_type = other DNA
ggtaccctccggcaaagtgg

SEQ ID NO: 122 - DNA, 20 bp, synthetic construct
  misc_feature 1..20: note = Synthetic polynucleotide
  source 1..20: mol_type = other DNA
tggccaccacaacgattgtc

SEQ ID NO: 123 - DNA, 20 bp, synthetic construct
  misc_feature 1..20: note = Synthetic polynucleotide
  source 1..20: mol_type = other DNA
ggcgtactccaaaagtgttt

SEQ ID NO: 124 - DNA, 20 bp, synthetic construct
  misc_feature 1..20: note = Synthetic polynucleotide
  source 1..20: mol_type = other DNA
ccacagctggtacatccacg

SEQ ID NO: 125 - DNA, 20 bp, synthetic construct
  misc_feature 1..20: note = Synthetic polynucleotide
  source 1..20: mol_type = other DNA
gttgtggatactcacttgct

SEQ ID NO: 126 - DNA, 20 bp, synthetic construct
  misc_feature 1..20: note = Synthetic polynucleotide
  source 1..20: mol_type = other DNA
ggactggagaaccatcatcc

SEQ ID NO: 127 - DNA, 20 bp, synthetic construct
  misc_feature 1..20: note = Synthetic polynucleotide
  source 1..20: mol_type = other DNA
tggtaagctctacccactcc

SEQ ID NO: 128 - DNA, 20 bp, synthetic construct
  misc_feature 1..20: note = Synthetic polynucleotide
  source 1..20: mol_type = other DNA
tagttcctcgttttttacat

SEQ ID NO: 129 - DNA, 20 bp, synthetic construct
  misc_feature 1..20: note = Synthetic polynucleotide
  source 1..20: mol_type = other DNA
ctccttcacaatgcttggaa

SEQ ID NO: 130 - DNA, 20 bp, synthetic construct
  misc_feature 1..20: note = Synthetic polynucleotide
  source 1..20: mol_type = other DNA
gctaaagaaccattccggca

SEQ ID NO: 131 - DNA, 20 bp, synthetic construct
  misc_feature 1..20: note = Synthetic polynucleotide
  source 1..20: mol_type = other DNA
ttctgcacctatccgagcta

SEQ ID NO: 132 - DNA, 20 bp, synthetic construct
  misc_feature 1..20: note = Synthetic polynucleotide
  source 1..20: mol_type = other DNA
ctaaagaaccattccggcag

SEQ ID NO: 133 - DNA, 20 bp, synthetic construct
  misc_feature 1..20: note = Synthetic polynucleotide
  source 1..20: mol_type = other DNA
cacaacgattgtcaggctct

SEQ ID NO: 134 - DNA, 20 bp, synthetic construct
  misc_feature 1..20: note = Synthetic polynucleotide
  source 1..20: mol_type = other DNA
caagagcctgacaatcgttg

SEQ ID NO: 135 - DNA, 20 bp, synthetic construct
  misc_feature 1..20: note = Synthetic polynucleotide
  source 1..20: mol_type = other DNA
gtatccacaactacaccatc

SEQ ID NO: 136 - DNA, 20 bp, synthetic construct
  misc_feature 1..20: note = Synthetic polynucleotide
  source 1..20: mol_type = other DNA
ctcacctccactttgccgga

SEQ ID NO: 137 - DNA, 20 bp, synthetic construct
  misc_feature 1..20: note = Synthetic polynucleotide
  source 1..20: mol_type = other DNA
cctcgtggatgtaccagctg

SEQ ID NO: 138 - DNA, 20 bp, synthetic construct
  misc_feature 1..20: note = Synthetic polynucleotide
  source 1..20: mol_type = other DNA
ctgaccagatggtgtagttg

SEQ ID NO: 139 - DNA, 20 bp, synthetic construct
  misc_feature 1..20: note = Synthetic polynucleotide
  source 1..20: mol_type = other DNA
tggatactcacttgcttggc

SEQ ID NO: 140 - DNA, 20 bp, synthetic construct
  misc_feature 1..20: note = Synthetic polynucleotide
  source 1..20: mol_type = other DNA
accacctagtattgctacca

SEQ ID NO: 141 - DNA, 20 bp, synthetic construct
  misc_feature 1..20: note = Synthetic polynucleotide
  source 1..20: mol_type = other DNA
tataagagggtcttcgtgaa

SEQ ID NO: 142 - DNA, 20 bp, synthetic construct
  misc_feature 1..20: note = Synthetic polynucleotide
  source 1..20: mol_type = other DNA
tcttcaatcttgggaacacc

SEQ ID NO: 143 - DNA, 20 bp, synthetic construct
  misc_feature 1..20: note = Synthetic polynucleotide
  source 1..20: mol_type = other DNA
ctagtctgatatatgctagt

SEQ ID NO: 144 - DNA, 20 bp, synthetic construct
  misc_feature 1..20: note = Synthetic polynucleotide
  source 1..20: mol_type = other DNA
gccatggtagcaatactagg

SEQ ID NO: 145 - DNA, 20 bp, synthetic construct
  misc_feature 1..20: note = Synthetic polynucleotide
  source 1..20: mol_type = other DNA
tatgctagttggtactgcca

SEQ ID NO: 146 - DNA, 20 bp, synthetic construct
  misc_feature 1..20: note = Synthetic polynucleotide
  source 1..20: mol_type = other DNA
actgccatggtagcaatact

SEQ ID NO: 147 - DNA, 20 bp, synthetic construct
  misc_feature 1..20: note = Synthetic polynucleotide
  source 1..20: mol_type = other DNA
caacacgcaaacgagagctg

SEQ ID NO: 148 - DNA, 20 bp, synthetic construct
  misc_feature 1..20: note = Synthetic polynucleotide
  source 1..20: mol_type = other DNA
ccagaacaagaagcttatag

SEQ ID NO: 149 - DNA, 20 bp, synthetic construct
  misc_feature 1..20: note = Synthetic polynucleotide
  source 1..20: mol_type = other DNA
gcatatatcagactagcacg

SEQ ID NO: 150 - DNA, 20 bp, synthetic construct
  misc_feature 1..20: note = Synthetic polynucleotide
  source 1..20: mol_type = other DNA
gctcatgtgcagactcagac

SEQ ID NO: 151 - DNA, 20 bp, synthetic construct
  misc_feature 1..20: note = Synthetic polynucleotide
  source 1..20: mol_type = other DNA
gtaagttggcatgtcaccag

SEQ ID NO: 152 - DNA, 20 bp, synthetic construct
  misc_feature 1..20: note = Synthetic polynucleotide
  source 1..20: mol_type = other DNA
ggtgtatcctcagcacggag

SEQ ID NO: 153 - DNA, 20 bp, synthetic construct
  misc_feature 1..20: note = Synthetic polynucleotide
  source 1..20: mol_type = other DNA
ggacttccagcactgcaact

SEQ ID NO: 154 - DNA, 20 bp, synthetic construct
  misc_feature 1..20: note = Synthetic polynucleotide
  source 1..20: mol_type = other DNA
cctactgctgctttgccaca

SEQ ID NO: 155 - DNA, 20 bp, synthetic construct
  misc_feature 1..20: note = Synthetic polynucleotide
  source 1..20: mol_type = other DNA
tagcatatatcagactagca

SEQ ID NO: 156 - DNA, 20 bp, synthetic construct
  misc_feature 1..20: note = Synthetic polynucleotide
  source 1..20: mol_type = other DNA
tcctactgctgctttgccac

SEQ ID NO: 157 - DNA, 20 bp, synthetic construct
  misc_feature 1..20: note = Synthetic polynucleotide
  source 1..20: mol_type = other DNA
tttcaggcgtcctatagaag

SEQ ID NO: 158 - DNA, 20 bp, synthetic construct
  misc_feature 1..20: note = Synthetic polynucleotide
  source 1..20: mol_type = other DNA
gaactgtcctctgatgtgcc

SEQ ID NO: 159 - DNA, 20 bp, synthetic construct
  misc_feature 1..20: note = Synthetic polynucleotide
  source 1..20: mol_type = other DNA
gtctgggtgtatcctcagca

SEQ ID NO: 160 - DNA, 20 bp, synthetic construct
  misc_feature 1..20: note = Synthetic polynucleotide
  source 1..20: mol_type = other DNA
aagctgcccgggagtgtcgc

SEQ ID NO: 161 - DNA, 20 bp, synthetic construct
  misc_feature 1..20: note = Synthetic polynucleotide
  source 1..20: mol_type = other DNA
ctgtctgcagaagcccatta

SEQ ID NO: 162 - DNA, 20 bp, synthetic construct
  misc_feature 1..20: note = Synthetic polynucleotide
  source 1..20: mol_type = other DNA
ctggctaacaatggtaccga

SEQ ID NO: 163 - DNA, 20 bp, synthetic construct
  misc_feature 1..20: note = Synthetic polynucleotide
  source 1..20: mol_type = other DNA
cagctgtactagagttacgg

SEQ ID NO: 164 - DNA, 20 bp, synthetic construct
  misc_feature 1..20: note = Synthetic polynucleotide
  source 1..20: mol_type = other DNA
tggagttggcaccgttacag

SEQ ID NO: 165 - DNA, 20 bp, synthetic construct
  misc_feature 1..20: note = Synthetic polynucleotide
  source 1..20: mol_type = other DNA
tactgtagaatggtagtacc

SEQ ID NO: 166 - DNA, 20 bp, synthetic construct
  misc_feature 1..20: note = Synthetic polynucleotide
  source 1..20: mol_type = other DNA
gacttatcttctgatgcacc

SEQ ID NO: 167 - DNA, 20 bp, synthetic construct
  misc_feature 1..20: note = Synthetic polynucleotide
  source 1..20: mol_type = other DNA
taactgattcccaaaagcga

SEQ ID NO: 168 - DNA, 20 bp, synthetic construct
  misc_feature 1..20: note = Synthetic polynucleotide
  source 1..20: mol_type = other DNA
gttctcggctccagattcca

SEQ ID NO: 169 - DNA, 20 bp, synthetic construct
  misc_feature 1..20: note = Synthetic polynucleotide
  source 1..20: mol_type = other DNA
gggcttgaactgtcatttgt

SEQ ID NO: 170 - DNA, 20 bp, synthetic construct
  misc_feature 1..20: note = Synthetic polynucleotide
  source 1..20: mol_type = other DNA
tcaagtccatggagtcattc

SEQ ID NO: 171 - DNA, 20 bp, synthetic construct
  misc_feature 1..20: note = Synthetic polynucleotide
  source 1..20: mol_type = other DNA
tggctaacaatggtaccgat

SEQ ID NO: 172 - DNA, 20 bp, synthetic construct
  misc_feature 1..20: note = Synthetic polynucleotide
  source 1..20: mol_type = other DNA
ggctaacaatggtaccgatg

SEQ ID NO: 173 - DNA, 20 bp, synthetic construct
  misc_feature 1..20: note = Synthetic polynucleotide
  source 1..20: mol_type = other DNA
agctgtactagagttacggt

SEQ ID NO: 174 - DNA, 20 bp, synthetic construct
  misc_feature 1..20: note = Synthetic polynucleotide
  source 1..20: mol_type = other DNA
tatactgtccactgctagtt

SEQ ID NO: 175 - DNA, 20 bp, synthetic construct
  misc_feature 1..20: note = Synthetic polynucleotide
  source 1..20: mol_type = other DNA
ctctagtacagctgcccaat

SEQ ID NO: 176 - DNA, 20 bp, synthetic construct
  misc_feature 1..20: note = Synthetic polynucleotide
  source 1..20: mol_type = other DNA
gacttgaactgtctgcccat

SEQ ID NO: 177 - DNA, 20 bp, synthetic construct
  misc_feature 1..20: note = Synthetic polynucleotide
  source 1..20: mol_type = other DNA
actctagtacagctgcccaa

SEQ ID NO: 178 - DNA, 20 bp, synthetic construct
  misc_feature 1..20: note = Synthetic polynucleotide
  source 1..20: mol_type = other DNA
tcttcaatccttggcactcc

SEQ ID NO: 179 - DNA, 20 bp, synthetic construct
  misc_feature 1..20: note = Synthetic polynucleotide
  source 1..20: mol_type = other DNA
acttgaactgtctgcccatt

SEQ ID NO: 180 - DNA, 20 bp, synthetic construct
  misc_feature 1..20: note = Synthetic polynucleotide
  source 1..20: mol_type = other DNA
ccactgctagtttggtaaat

SEQ ID NO: 181 - DNA, 20 bp, synthetic construct
  source 1..20: mol_type = other DNA
tgatggtgttcagggactgc

SEQ ID NO: 182 - DNA, 21 bp, synthetic construct
  source 1..21: mol_type = other DNA
actggtgacatgccaacttac

SEQ ID NO: 183 - DNA, 20 bp, synthetic construct
  source 1..20: mol_type = other DNA
gctgtaactggagatgacac

SEQ ID NO: 184 - DNA, 20 bp, synthetic construct
  source 1..20: mol_type = other DNA
gcttagtggtaagatcgagc

SEQ ID NO: 185 - protein, 218 aa, synthetic construct
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWV

SEQ ID NO: 186 - protein, 191 aa, synthetic construct
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 187 - protein, 27 aa, synthetic construct
FWVLVVVGGVLACYSLLVTVAFIIFWV

SEQ ID NO: 188 - protein, 260 aa, synthetic construct
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 189 - protein, 172 aa, synthetic construct
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 190 - protein, 19 aa, synthetic construct
MARPHPWWLCVLGTLVGLS

SEQ ID NO: 191 - protein, 113 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 192 - protein, 41 aa, synthetic construct
RSKRSRLLHSDYMNMTPRRPGPTRKHYQPYAPPRDFAAYRS

SEQ ID NO: 193 - protein, 24 aa, synthetic construct
LCARPRRSPAQEDGKVYINMPGRG